(19) United States
(12) Patent Application Publication
(10) Pub. No.: US 2025/0188041 A1
(43) Pub. Date: Jun. 12, 2025

(54) NEW COMPOUNDS AND TREATMENT OF RENAL CELL CARCINOMA

(71) Applicant: Trustlife Global Inc., Wilmington, DE (US)

(72) Inventors: Adil MARDINOGLU, Göteborg (SE); Xiangyu LI, Stockholm (SE); Younos BOUZIAN, Ümraniye/Istanbul (TR); Mohamed EL HAFI, Ümraniye/Istanbul (TR); Negar PARVIZI, Ümraniye/Istanbul (TR)

(21) Appl. No.: 18/842,111

(22) PCT Filed: Mar. 2, 2023
(86) PCT No.: PCT/EP2023/055381

§ 371 Date: Aug. 28, 2024

(30) Foreign Application Priority Data
Mar. 2, 2022 (EP) 22159838.6

(51) Int. Cl. C07D 239/48; A61K 31/506; A61K 31/5377; A61P 35/00; C07D 401/12; C07D 403/12; C07D 413/12

(52) U.S. Cl. CPC C07D 239/48; A61K 31/506; A61K 31/5377; A61P 35/00; C07D 401/12; C07D 403/12; C07D 413/12

(57) ABSTRACT

There is provided a compound according to formula (I) or a pharmaceutically acceptable salt or prodrug thereof.

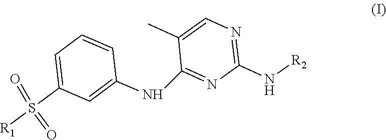

### TECHNICAL FIELD

The present disclosure relates to the field of treatment of renal cell carcinoma.

### BACKGROUND

Clear cell renal cell carcinoma (ccRCC) is the most common histological subtype of renal cell carcinoma (RCC) and accounts for 70% of all RCC cases [1]. Surgery (radical or partial nephrectomy) is the standard primary treatment for patients with localized tumors. The first-line and second-line target therapy options for patients with relapse after nephrectomy or advanced stage tumor include tyrosine kinase inhibitors (axitinib, sorafenib, pazopanib, and sunitinib, etc.), mTOR inhibitors (everolimus and tesirolimus), and monoclonal antibodies against VEGF, PD-1 or PD-L1 (bevacizumab, pembrolizumab and avelumab, etc.). However, the National Comprehensive Cancer Network (NCCN, version: 1.2022) has reported that the response rates of the single-agent or combinatory regimens based on these drugs range from 6% to 50% in different clinical trials [2]. Moreover, the average duration of disease control with these drugs is only 8-9 months for the first-line setting and 5-6 months for the second-line setting [3]. Therefore, there is a need to discover more tolerated and effective drugs to widen the options for single-agent or combinatory regimens for ccRCC patients.

### SUMMARY

Computational drug repositioning based on systems biology methods has become a powerful tool to identify potential drug-target interactions and drug-disease interactions [4]. The advantage of drug repositioning is that the pharmacology and safety of the repositioned drugs have been well-characterized, dramatically decreasing the cost and duration taken by traditional drug development and reducing the risk of attrition in clinical phases [5,6]. In general, current drug repositioning strategies can be classified into drug-based, disease-based and profile-based [7]. Usually, drug-based and disease-based approaches are conducted by comparing drug-drug or disease-disease similarity or applying existing drug treatment knowledge to predict new disease-drug associations [8,9]. In contrast, profile-based approaches are conducted by analyzing the high-throughput multi-omics data associated with diseases and drugs, which do not rely on prior knowledge about a particular drug or disease and have increased ability to discover new drug-disease pairs [7].

Recently, several studies have employed profile-based repositioning methods to identify potentially valuable drugs for the treatment of ccRCC. A widely used method is selecting the drug that has a reversed effect on the disease signature genes [10,11]. The idea of this method is that if the perturbation of gene expression induced by a drug (drug-perturbed signatures) is negatively correlated with the dysregulation in the tumor tissues compared to normal tissues (disease-specific signatures), this drug turns out to have therapeutic value for this tumor type. During the application of the above approach, ConnectivityMap (CMap) [12] is the most commonly used drug-perturbed gene expression data source, and it has been recently updated and integrated into the LINCS Data Portal [13]. To date, the LINCS data portal includes more than three million gene expression profiles associated with more than 20,000 drugs, gene overexpression and knockdown in up to more than 200 cell lines [13]. However, a drug repurposed in this way is supposed to have multiple gene targets mixed by oncogenes and passenger genes, limiting the identification and validation of the key targets and mechanisms of drug effect.

By starting from the target prediction and then repurposing the existed inhibitor of the target genes for cancer treatment this problem has been avoided. In a previous study, a list of candidate target genes which are essential for the ccRCC tumor cell growth by the genome-scale metabolic model analysis were identified [14]. Among these essential genes, three genes, SOAT1, CRLS1 and ACACB, whose inhibition is not toxic in the 32 major normal human tissues have been filtered out by performing an in silico toxicity test for each essential gene, as the final targetable genes. Finally, a well-known inhibitor of SOAT1, mitotane, was repurposed and its drug efficacy for the treatment of ccRCC was validated. However, one limitation of this study is that a systematic drug repositioning approach is needed to discover more potentially effective drugs that could inhibit SOAT1. In addition, the list of candidate target genes need to be extended and this extension should not be limited to the metabolically important genes, which covers only around 20-25% of the human genome (as reported in the human metabolic model [15]), but should also include non-metabolic genes. Accordingly, gene co-expression network (GCN) analysis that applies all possible human genes has been used to identify the key genes, its neighbors and functionally related biological functions [5,16].

In the study of the present disclosure, an integrated strategy that involved disease-target prediction and drug-target prediction was applied. First, a set of robust ccRCC signature genes whose expression was significantly associated with patients' survival outcomes were extracted. Functional enrichment analysis showed that these genes were significantly enriched in the cell division, cell cycle, DNA replication, angiogenesis, cell migration and cell differentiation pathways, all of which are well-known hallmarks in cancer [17]. Second, two types of molecular modules that significantly enriched with the unfavorable and favorable signatures were identified based on the GCN analysis of the transcriptomic data of ccRCC tissues. Third, four target genes, i.e. BUB1B, RRM2, ASF1B and CCNB2, showing high centrality in the modules were extracted. Next, a drug repositioning approach based on the analysis of shRNA-perturbed and drug-perturbed transcriptomics data from the LINCS data portal was developed and the three most effective drugs for each target were repurposed. Finally, the drug effects were tested in Caki-1 cells. In these tests, the drug TG-101209 was shown to be effective.

Next, the present inventors realized that the molecular structure of TG-101209 could be tuned in order to find alternative small molecules with similar or improved effect against ccRCC.

According to a first aspect, there is thus provided a compound according to formula (I)

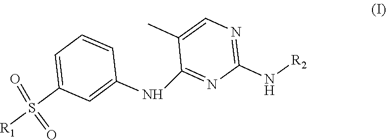

or a pharmaceutically acceptable salt or prodrug thereof wherein:
R1 is selected from

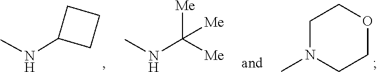

R2 is

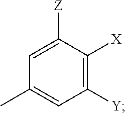

Z is —H, —OMe or —SO₂—NH₂;
X is —H, -Me,

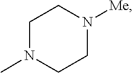

-NHC(O)—Ar or —C(O)NH—Ar, wherein —Ar is an optionally substituted aryl; and
Y is —H, —OMe or

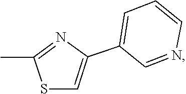

provided that the compound is not TG-101209.

A compound according to formula (I) exhibits cytotoxicity towards renal cell carcinoma cells and reduce the cell viability of the treated cells. The compounds of the present invention are hence suitable for use in the treatment of renal cell carcinoma, such as clear cell renal cell carcinoma (ccRCC).

In one embodiment, Y and Z is —H, X is —C(O)NH—Ar or —NHC(O)—Ar, preferably —C(O)NH—Ar, and the optionally substituted aryl, —Ar, has one or two rings. The optionally substituted aryl, —Ar, may be chosen from the group consisting of optionally substituted benzyl, naphthyl, quinolyl, indolyl and purinyl.

In one embodiment, —Ar is selected from:

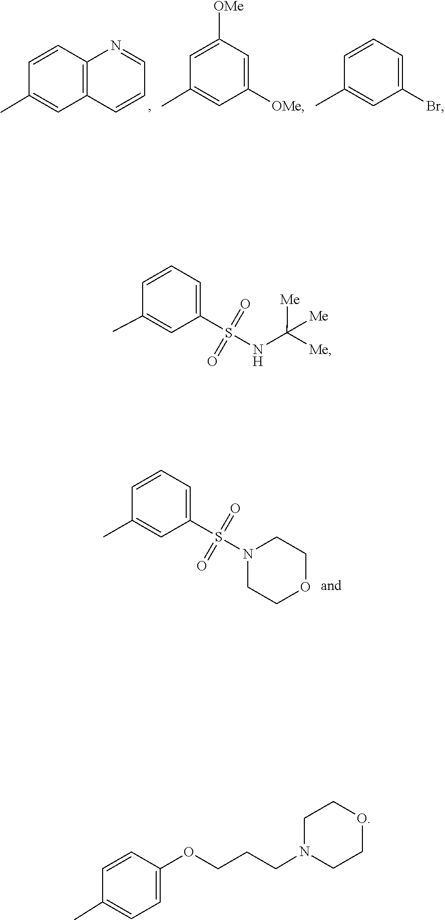

Compounds M101A, M101B, M101D, M101F, M101G and M101H in the Examples section are included in this embodiment and treatment using the compounds in this embodiment is associated with a particularly low cell viability.

In one embodiment, —Ar is

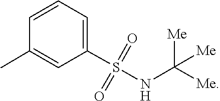

Compound M101D in the Examples section is included in this embodiment and treatment of cells using the compounds in this embodiment is associated with a particularly low cell viability.

In one embodiment, X is -Me, Y is —H and Z is-SO₂NH₂. Compound M66 in the Examples section is included in this embodiment and treatment of cells using the compounds in this embodiment is related to a particularly low cell viability and reduction of the expression levels of BUB1B.

In one embodiment, R2 is

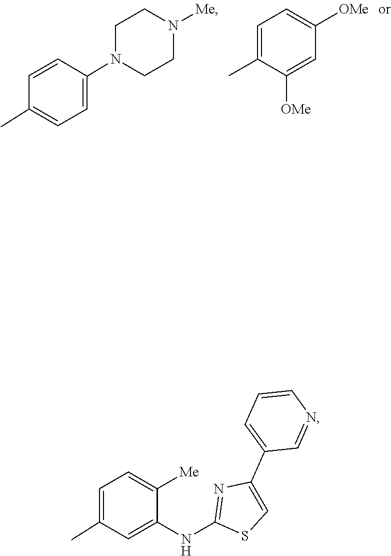 [SMILES: Cc1ccc(N2CCN(C)CC2)cc1 ; COc1ccc(C)c(OC)c1 ; Cc1ccc(C)c(Nc2nc(-c3cccnc3)cs2)c1]

preferably R2 is

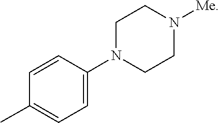

Compounds M5, M7, M79 and M88 in the Examples section are included in this embodiment and treatment of cells using the compounds of this embodiment is associated with an especially low cell viability and a reduction of the expression levels of BUB1B.

In one embodiment R1 is

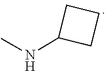

In this embodiment, Z is preferably —H or —SO₂—NH₂ and/or Y is preferably —H or

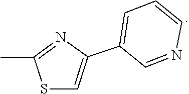

Compound M5 in the Examples section is included in this embodiment and treatment of cells with the compounds in this embodiment are associated with a particularly low cell viability and a reduction of the expression levels of BUB1B.

In another embodiment, R1 is

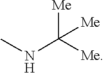

Compounds M66, M79, M88, M101A, M101B, M101D, M101F, M101G and M101H in the examples section are included in this embodiment and treatment of cells using the compounds of this embodiment is associated with a significantly reduced cell viability.

In one embodiment, R1 is

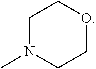

Compound M7 in the Examples section is included in this embodiment and the treatment of cells using the compounds of this embodiment is associated with a reduced cell viability.

In one embodiment, compounds according to formula (II) or (III) are excluded from the invention:

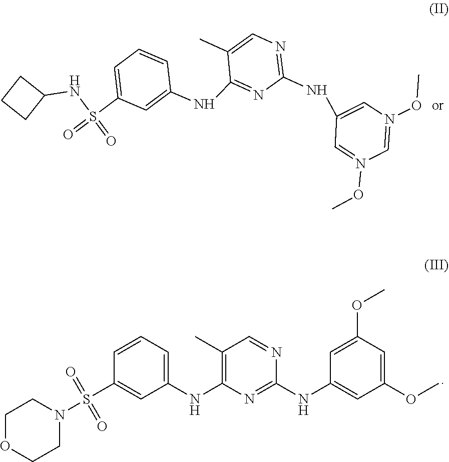

Cells treated with these compounds exhibit a reduction in cell viability. However, other compounds show a greater effect and are hence more preferred.

Accordingly, it is preferred that R1 is

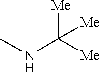

when Z and Y are both —OMe.

In a preferred embodiment of the first aspect, the compound is M5, M66, M101A, M101B or M101D or a pharmaceutically acceptable salt or prodrug thereof. The structures of M5, M66, M101A, M101B and M101D are disclosed below, e.g. in table 1.

In a particularly preferred embodiment of the first aspect, the compound is M5 or M101D or a pharmaceutically acceptable salt or prodrug thereof.

According to a second aspect, a pharmaceutical composition comprising a compound according to the first aspect is provided.

According to a third aspect, a compound or a pharmaceutical composition according to the first or second aspect including compound TG-101209 for use in a method of treatment of a renal cell carcinoma, such as clear cell renal cell carcinoma (ccRCC) is provided. The ccRCC may be stage I-III ccRCC, preferably it is stage I or II ccRCC.

Hence the compound TG-101209 is excluded from the scope of the first and the second aspect, but not from the scope of the third aspect.

In one embodiment, the method of treatment further comprises administration of a drug selected from the group consisting of tyrosine kinase inhibitors (e.g. axitinib, sorafenib, pazopanib and sunitinib), mTOR inhibitors (e.g. everolimus and tesirolimus) and monoclonal antibodies against VEGF (e.g. bevacizumab). The administration of these drugs together with the compound or pharmaceutical composition according to the first or second aspect may have a synergistic effect on the treatment of ccRCC.

In another embodiment, the method of treatment further comprises administration of:
i) panobinostat; and/or
ii) NVP-TAE684 or withaferin-a.

The combination of this embodiment is expected to have a synergistic effect (as further discussed below).

### BRIEF DESCRIPTION OF THE DRAWINGS

FIG. 1 shows a topology analysis of the unfavorable and favorable modules. A) Spearman correlation of degree, betweenness or closeness centralities of genes from M11 in the TCGA cohort and M3 in the Japanese cohort. Coef, Spearman correlation coefficient. B) Spearman correlation of degree, betweenness, and closeness centralities between genes from the merged module M1/2/3/4/18 in the TCGA cohort and M1 in the Japanese cohort. Coef, the Spearman correlation coefficient. The gray area indicates the 95% confidence interval for the prediction of the linear model. C) The top 10 genes with the highest degree values in M11 from the TCGA cohort and M3 from the Japanese cohort, respectively. The common genes between the two cohorts are ASF1B, RRM2, BUB1B, CEP55 and TPX2. D) The top 10 genes with the highest degree centrality in M1/2/3/4/18 from the TCGA cohort and M1 from the Japanese cohort, respectively. The common genes between the two cohorts are MMRN2, TCF4, ARAP3 and ERG.

FIG. 2 shows the alteration of hub genes in tumor tissues or cell lines. A) Box plots showing the mRNA expression levels (TPM values) of unfavorable hub genes in TCGA tumors compared to normal tissues. The differential expression analysis was performed by DESeq2. * represents FDR<0.05. B) Box plots showing the mRNA expression levels (TPM values) of favorable hub genes in TCGA tumor tissues compared to normal tissues. * represents FDR<0.05. C) The log 2 fold changes between the expression value of a hub gene in each TCGA tumor sample and the average expression value of this gene in TCGA normal samples. The x-axis represents individual tumor samples. Log 2(FC), log 2 fold change. D) The IHC images of hub genes in normal tissues and tumor tissues. The images related to the same gene were conducted by the same antibody, which was described in the Method section. Scale bar, 50 μm. E) The essential scores of hub genes in 16 ccRCC cell lines. More negative scores indicate more essential for tumor cell survival and proliferation. F) The mRNA expression levels (TPM values) of hub genes in the Caki-1 cell line.

FIG. 3 shows drug repositioning for the target genes. Box plots showing the three most effective drugs for each target gene. Each point represents the similarity (calculated as spearman correlation coefficient) between the repurposed drug perturbed effects and shRNA knockdown perturbed effects on cells. The bottom and top of the boxes represent the 25^{th} and 75^{th} percentiles, respectively. The whiskers represent the minimum and maximum values that are not outliers. The central band represents the median value.

FIG. 4 shows validation of target genes and drug effects. A-E) Western blots showing the protein levels of target genes in Caki-1 with siRNA transfection and negative control and bar plots showing the change of cell viability. F-H) Western plots showing that the protein levels of target genes were inhibited by the treatment of their corresponding target drugs and bar plots showing the change of cell viability. The cells in the negative control group were only treated by DMSO. The change of cell viability between the two groups was compared by two-sided T-test. * represents P<0.05. ** represents P<0.01, *** represents P<0.001. Error bars represent standard deviation.

FIG. 5 shows the synthesis of (S)—N-(1-(4-bromophenyl)ethyl)-3-((5-methyl-2-((4-(4-methylpiperazin-1-yl)phenyl)amino)pyrimidin-4-yl)amino)benzenesulfonamide (M1). A) shows the synthesis of intermediate (S)-3-bromo-N-(1-(4-bromophenyl)ethyl)benzenesulfonamide (CIM1). B) shows the synthesis of intermediate 5-methyl-N2-(4-(4-methylpiperazin-1-yl)phenyl)pyrimidine-2,4-diamine (TG01). C) shows the final synthesis step of (S)—N-(1-(4-bromophenyl)ethyl)-3-((5-methyl-2-((4-(4-methylpiperazin-1-yl)phenyl)amino)pyrimidin-4-yl)amino)benzenesulfonamide (M1).

FIG. 6 shows the synthesis of 3-((5-methyl-2-((4-(4-methylpiperazin-1-yl)phenyl)amino)pyrimidin-4-yl)amino)-N-phenylbenzenesulfonamide (M3). A) shows the synthesis of intermediate 3-bromo-N-phenylbenzenesulfonamide (CIM3). B) shows the final synthesis step of 3-((5-methyl-2-((4-(4-methylpiperazin-1-yl)phenyl)amino)pyrimidin-4-yl)amino)-N-phenylbenzenesulfonamide (M3).

FIG. 7 shows the synthesis of N-cyclobutyl-3-((5-methyl-2-((4-(4-methylpiperazin-1-yl)phenyl)amino)pyrimidin-4-yl)amino)benzenesulfonamide (M5). A) shows the synthesis of intermediate 3-bromo-N-cyclobutylbenzenesulfonamide (CIM5). B) shows the final synthesis step of N-cyclobutyl-3-((5-methyl-2-((4-(4-methylpiperazin-1-yl)phenyl)amino)pyrimidin-4-yl)amino)benzenesulfonamide (M5).

FIG. 8 shows the synthesis of 5-methyl-N2-(4-(4-methylpiperazin-1-yl)phenyl)-N4-(3-(morpholinosulfonyl)phenyl)pyrimidine-2,4-diamine (M7). A) show the synthesis of 4-((3-bromophenyl)sulfonyl)morpholine (CIM7). B) show the final synthesis step of 5-methyl-N2-(4-(4-methylpiperazin-1-yl)phenyl)-N4-(3-(morpholinosulfonyl)phenyl)pyrimidine-2,4-diamine (M7).

FIG. 9 shows the synthesis of 5-methyl-N4-(5-methyl-4-(4-(trifluoromethyl)phenyl)pyrimidin-2-yl)-N2-(4-(4-methylpiperazin-1-yl)phenyl)pyrimidine-2,4-diamine (M94). A) shows the synthesis of intermediate 2-chloro-5-methyl-4-(4-(trifluoromethyl)phenyl)pyrimidine (Int 4). B) shows the final synthesis step of 5-methyl-N4-(5-methyl-4-(4-(trifluoromethyl)phenyl)pyrimidin-2-yl)-N2-(4-(4-methylpiperazin-1-yl)phenyl)pyrimidine-2,4-diamine (M94).

FIG. 10 shows the synthesis of N-(5-methyl-2-((4-(4-methylpiperazin-1-yl)phenyl)amino)pyrimidin-4-yl)acetamide (B14).

FIG. 11 shows the synthesis of N-(5-methyl-2-((4-(4-methylpiperazin-1-yl)phenyl)amino)pyrimidin-4-yl)quinoline-2-carboxamide (B2).

FIG. 12 shows the synthesis of N-(tert-butyl)-3-((2-((6-(4-chlorophenoxy)pyridin-3-yl)amino)-5-methylpyrimidin-4-yl)amino)benzenesulfonamide (M24). A) shows the synthesis of intermediate 3-bromo-N-(tert-butyl)benzenesulfonamide (A). B) shows the synthesis of N-(tert-butyl)-3-((2-chloro-5-methylpyrimidin-4-yl)amino)benzenesulfonamide (TGo). C) shoes the final synthesis step of N-(tert-butyl)-3-((2-((6-(4-chlorophenoxy)pyridin-3-yl)amino)-5-methylpyrimidin-4-yl)amino)benzenesulfonamide (M24).

FIG. 13 shows the synthesis of N-(tert-butyl)-3-((5-methyl-2-((3-(morpholinosulfonyl)phenyl)amino)pyrimidin-4-yl)amino)benzenesulfonamide (M25)

FIG. 14 shows the synthesis of N-(tert-butyl)-3-((5-methyl-2-((4-(N-(5-methylisoxazol-3-yl)sulfamoyl)phenyl)amino)pyrimidin-4-yl)amino)benzenesulfonamide (M47).

FIG. 15 shows the synthesis of 3-((2-((3-((6,7-bis(2-methoxyethoxy)quinazolin-4-yl)amino)phenyl)ethynyl)-5-methylpyrimidin-4-yl)amino)-N-(tert-butyl)benzenesulfonamide (M48).

FIG. 16 shows the synthesis of 3,3′-((5-methylpyrimidine-2,4-diyl)bis(azanediyl))bis(N-(tert-butyl)benzenesulfonamide) (M55).

FIG. 17 shows the synthesis of 5-methyl-N2,N4-bis(3-(morpholinosulfonyl)phenyl)pyrimidine-2,4-diamine (M57). A) shows the Synthesis of 2-chloro-5-methyl-N-(3-(morpholinosulfonyl)phenyl)pyrimidin-4-amine (SM-M7). B) shows the final synthesis step of 5-methyl-N2,N4-bis(3-(morpholinosulfonyl)phenyl)pyrimidine-2,4-diamine (M57).

FIG. 18 shows the synthesis of N2-(6-(4-chlorophenoxy)pyridin-3-yl)-5-methyl-N4-(3-(morpholinosulfonyl) phenyl)pyrimidine-2,4-diamine (M58).

FIG. 19 shows the synthesis of N-(tert-butyl)-3-((5-methyl-4-((3-(morpholinosulfonyl)phenyl)amino)pyrimidin-2-yl)amino)benzenesulfonamide (M59).

FIG. 20 shows the synthesis of N-(tert-butyl)-3-((2-((2-methoxy-4-(4-(4-methylpiperazin-1-yl)piperidin-1-yl)phenyl)amino)-5-methylpyrimidin-4-yl)amino)benzenesulfonamide (M60).

FIG. 21 shows the synthesis of N-(tert-butyl)-3-((2-((2-(isopropylsulfonyl)phenyl)amino)-5-methylpyrimidin-4-yl)amino)benzenesulfonamide (M62).

FIG. 22 shows the synthesis of N-(tert-butyl)-3-((5-methyl-2-((3-(4-methyl-1H-imidazol-1-yl)-5-(trifluoromethyl)phenyl)amino)pyrimidin-4-yl)amino)benzenesulfonamide (M64).

FIG. 23 shows the synthesis of N-(tert-butyl)-3-((2-((2,4-dichloro-5-methoxyphenyl)amino)-5-methylpyrimidin-4-yl)amino)benzenesulfonamide (M69).

FIG. 24 shows the synthesis of N-(tert-butyl)-3-((2-((3,5-dimethoxyphenyl)amino)-5-methylpyrimidin-4-yl)amino)benzenesulfonamide (M79).

FIG. 25 shows the synthesis of N-(tert-butyl)-3-((5-methyl-2-((4-methyl-3-((4-(pyridin-3-yl)thiazol-2-yl)amino)phenyl)amino)pyrimidin-4-yl)amino)benzenesulfonamide (M88).

FIG. 26 shows the synthesis of 4-((4-((3-(N-(tert-butyl)sulfamoyl)phenyl)amino)-5-methylpyrimidin-2-yl)amino)benzoic acid (M101).

FIG. 27 shows the synthesis of tert-butyl 4-(4-((4-((3-(N-(tert-butyl)sulfamoyl)phenyl)amino)-5-methylpyrimidin-2-yl)amino)phenyl)piperazine-1-carboxylate (M102).

FIG. 28 shows the synthesis of N-(tert-butyl)-3-((5-methyl-2-((4-(3-morpholinopropoxy)phenyl)amino)pyrimidin-4-yl)amino)benzenesulfonamide (M103).

FIG. 29 shows the synthesis of N-(tert-butyl)-3-((5-methyl-2-((4-methyl-3-sulfamoylphenyl)amino)pyrimidin-4-yl)amino)benzenesulfonamide (M66).

FIG. 30 shows the synthesis of N-(tert-butyl)-3-((2-((5-((4-ethylpiperazin-1-yl)methyl)pyridin-2-yl)amino)-5-methylpyrimidin-4-yl)amino)benzenesulfonamide (M73).

FIG. 31 shows the synthesis of 4-((4-((3-(N-(tert-butyl)sulfamoyl)phenyl)amino)-5-methylpyrimidin-2-yl)amino)-N-(3,5-dimethoxyphenyl)benzamide (M101A).

FIG. 32 shows the synthesis of N-(3-bromophenyl)-4-((4-((3-(N-(tert-butyl)sulfamoyl)phenyl)amino)-5-methylpyrimidin-2-yl)amino)benzamide (M101B).

FIG. 33 shows synthesis of N-(3-(N-(tert-butyl)sulfamoyl)phenyl)-4-((4-((3-(N-(tert-butyl)sulfamoyl)phenyl)amino)-5-methylpyrimidin-2-yl)amino)benzamide (M101D).

FIG. 34 shows the synthesis of 4-((4-((3-(N-(tert-butyl)sulfamoyl)phenyl)amino)-5-methylpyrimidin-2-yl)amino)-N-(3-(morpholinosulfonyl)phenyl)benzamide (M101F).

FIG. 35 shows the synthesis of 4-((4-((3-(N-(tert-butyl)sulfamoyl)phenyl)amino)-5-methylpyrimidin-2-yl)amino)-N-(4-(3-morpholinopropoxy)phenyl)benzamide (M101G).

FIG. 36 shows the synthesis of 4-((4-((3-(N-(tert-butyl)sulfamoyl)phenyl)amino)-5-methylpyrimidin-2-yl)amino)-N-(quinolin-6-yl)benzamide (M101H).

FIG. 37 shows the synthesis of N2-(3,5-Dimethoxyphenyl)-5-methyl-N4-(3-(morpholinosulfonyl)phenyl)pyrimidine-2,4-diamine (M104).

FIG. 38 shows the synthesis of 5-methyl-N2-(4-methyl-3-((4-(pyridin-3-yl)thiazol-2-yl)amino)phenyl)-N4-(3-(morpholinosulfonyl)phenyl)pyrimidine-2,4-diamine (M105).

FIG. 39 shows the synthesis of 5-methyl-N2-(4-(3-morpholinopropoxy)phenyl)-N4-(3-(morpholinosulfonyl)phenyl)pyrimidine-2,4-diamine (M106).

FIG. 40 shows the synthesis of N-cyclobutyl-3-((2-((3,5-dimethoxyphenyl)amino)-5-methylpyrimidin-4-yl)amino)benzenesulfonamide (M108). A) shows the synthesis of intermediate 3-((2-chloro-5-methylpyrimidin-4-yl)amino)-N-cyclobutylbenzenesulfonamide (SM-M5). B) shows the final synthesis step of N-cyclobutyl-3-((2-((3,5-dimethoxyphenyl)amino)-5-methylpyrimidin-4-yl)amino)benzenesulfonamide (M108).

FIG. 41A-F shows the protein expression levels of BUB1B and β-actin of the Caki-1 cells treated with 5 μM of target drug for 1 day.

FIG. 42A-G shows the cell viability assessed by MTT assay of Caki-1 cells treated with 5 μM of target drug for 1 day.

FIG. 43A-D shows the IC₅₀ values of some of the target drugs.

FIG. 44 shows the DIC images of Caki-1 cells treated with 5 μM of target drug for 1 day (used for the MTT assay).

FIG. 45 shows the cell viability assessed by MTT assay of Caki-1 cells treated with 10 μM of the target drugs for 1 day.

FIG. 46 shows the cytotoxicity assessed by LDH assay of Caki-1 cells treated with 10 μM of the target drugs for 1 day.

FIG. 47 shows the the cell viability assessed by MTT assay and cytotoxicity assessed by LDH assay of Caki-1 cells treated with 10 μM of the target drugs for 1 day.

FIG. 48 shows the DIC images of Caki-1 cells treated with 10 μM of target drug for 1 day (used for the MTT assay).

### DETAILED DESCRIPTION

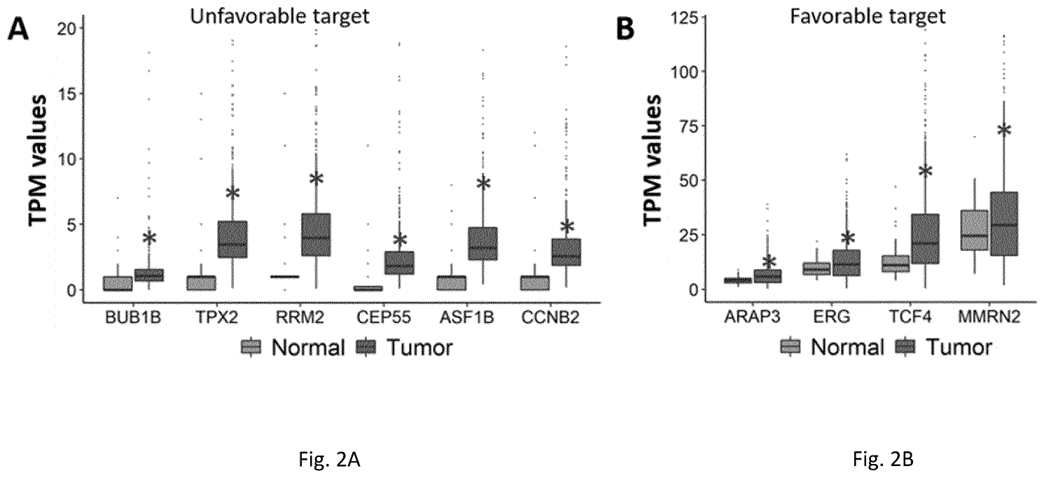

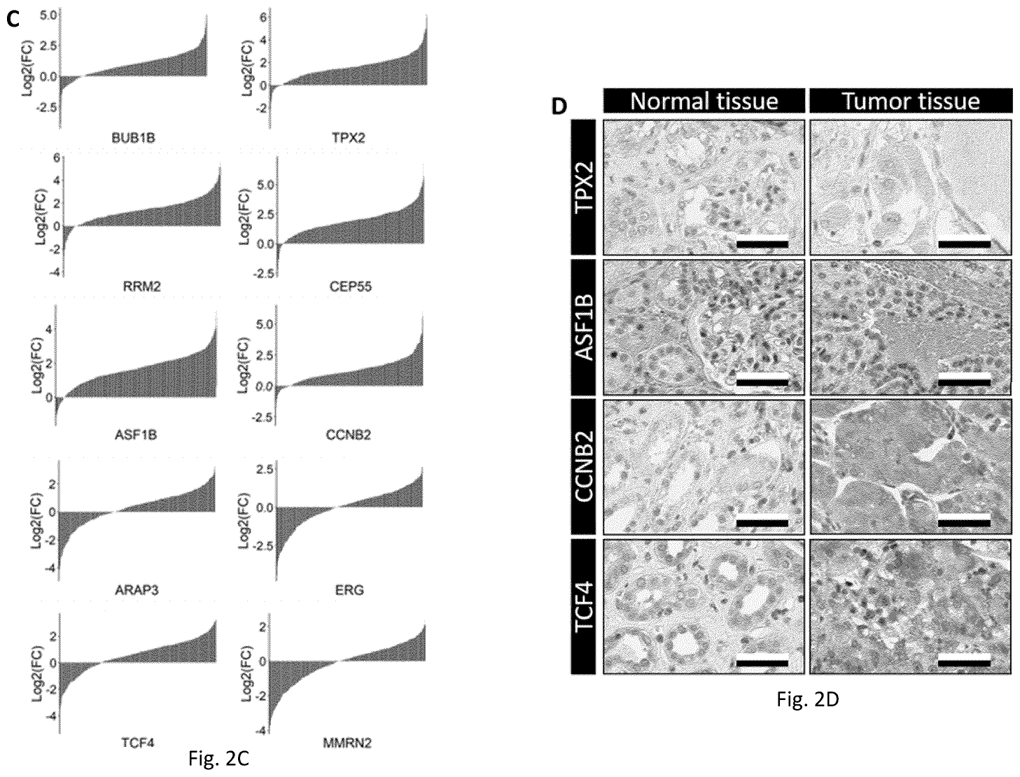

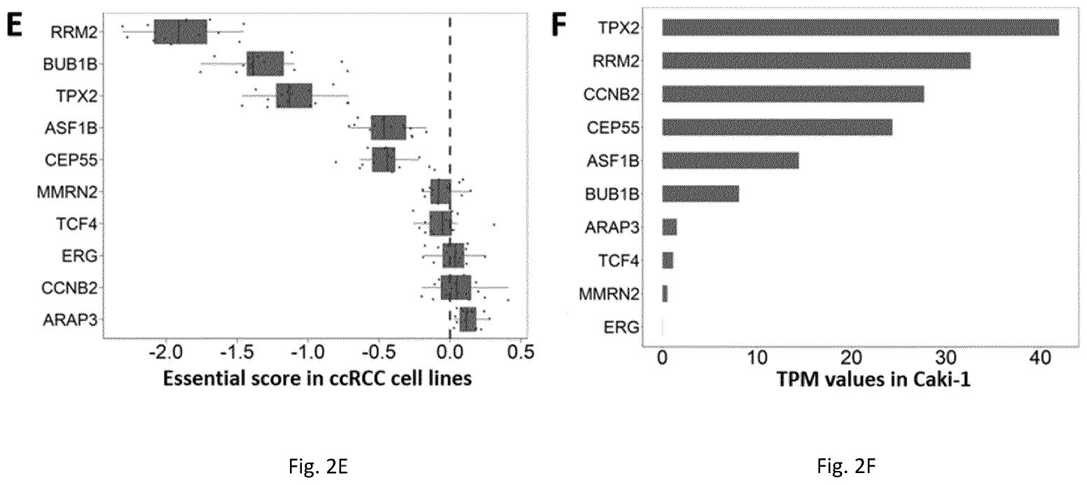

ccRCC is a heterogeneous tumor that has been stratified into several molecular subtypes characterized by distinct mRNA expression patterns and opposite survival outcomes of patients [14,32,33]. Different subtypes of patients respond differently to chemotherapy. Low response rates and drug resistance exacerbate the challenges in ccRCC therapy. Thus, there is an urgent need for discovering new treatment options for patients who cannot benefit from the commonly used chemotherapies. In the study behind the present disclosure, an integrated approach combining the target prediction and drug repositioning for ccRCC treatment was employed. As a result, four promising druggable target genes (i.e. BUB1B, RRM2, ASF1B and CCNB2), which encode the proteins involved in cell mitosis and cell cycle regulation were identified for treatment of ccRCC. BUB1B encodes a mitotic spindle checkpoint and exhibits a cell cycle dependent expression. It is undetectable in G1 and exhibits a gene expression peak in G2/M [34,35]. RRM2 encodes ribonucleotide reductase M2 subunit and it is responsible for the ribonucleotide deoxyribonucleotide conversion during the S phase of the cell cycle [36]. Thus, it is only expressed during the late G1/early S phase and degraded in late S phase [₃7]. CCNB2, encoding cyclin B2, is involved in the G2-M transition in eukaryotes by activating CDC2 kinase [38-40] and it also shows a gene expression peak in G2/M [35]. ASF1B, encoding one of the isoforms of the histone H3-H4 chaperone anti-silencing function 1, is necessary for cell proliferation and differently expressed in the cycling and non-cycling cells [41]. It has been reported that BUB1B is an independent prognostic marker [42], RRM2 is a drug resistance-related marker [43], ASF1B and CCNB2 are metastasis markers for ccRCC [44,45]. It is shown that these target genes are significantly upregulated in tumor tissues compared to the adjacent normal tissues. Moreover, broad coverage of upregulated alteration for each of these target genes was observed in ccRCC patients (FIG. 2C), implying that most patients may benefit from treatment of these target genes even disease states varied for individual patients.

Further, a drug repositioning approach using the correlation analysis between shRNA-perturbed and drug-perturbed signatures was developed and the three most effective drugs for each target were identified. The following inhibitory effects were validated using an in vitro model:
TG-101209 for targeting BUB1B;
NVP-TAE684 and withaferin-a for targeting RRM2; and
panobinostat for targeting ASF1B.

TG-101209, a JAK2 inhibitor, was originally developed for patients with myeloproliferative disorders who carry the JAK2V617F mutation [46]. It also inhibits the tumor cell growth in myeloma [47] and lung cancer [48] in in vitro or in vivo models.

NVP-TAE684, an ALK inhibitor, was originally designed to inhibit oncogenic ALK-rearranged fusion proteins (e.g., NPM-ALK) [49]. It has been reported that NVP-TAE684 suppressed the cell proliferation in pancreatic adenocarcinoma [50] and neuroblastoma [51], and reversed multidrug resistance in osteosarcoma [52].

Withaferin-a, a steroidal lactone derived from the medicinal plant Withania somnifera Dunal (Solanaceae), has a wide range of pharmacological activities, including cardioprotective, anti-inflammatory, and anti-angiogenesis [53]. It has been reported that withaferin-a induced cell apoptosis by inhibiting the expression or activation of STAT3 in ccRCC cells[54] and other cancer cells [55-57].

Panobinostat, a non-selective histone deacetylase inhibitor, is an FDA-approved oral drug to treat multiple myeloma [58].

Since all the three druggable targets, BUB1B, RRM2 and ASF1B, are cell cycle dependent genes, a combination of two or more of their corresponding repurposed drugs is expected to generate a synergistic effect on the inhibition of the tumor cell growth.

According to the present invention, TG-101209 is a promising drug candidate for the treatment of renal cell carcinoma. Therefore, the present inventors proceeded to identify alternative molecules by tuning the molecular structure of TG-101209. These compounds are defined by the first aspect and formula (I)

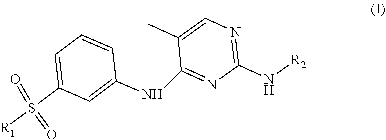

wherein:
R1 is selected from

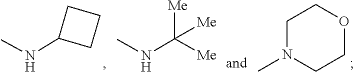

R2 is

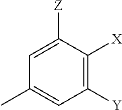

wherein Z is —H, —OMe or —SO₂—NH₂;
X is —H, -Me,

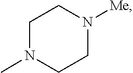

—NHC(O)—Ar or —C(O)NH—Ar, wherein —Ar is an optionally substituted aryl; and
Y is —H, —OMe or

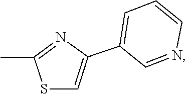

provided that the compound is not TG-101209.

The compounds according to formula (I) exhibit cytotoxicity towards renal cell carcinoma cells and reduce the cell viability of the treated cells. The compounds are hence suitable for use in the treatment of renal cell carcinoma such as clear cell renal cell carcinoma (ccRCC).

The compounds according to the first aspect target BUB1B, as the compounds are derivatives of TG-101209. These compounds may thereby be combined with one or more of NVP-TAE684 (targets RRM2), withaferin-a (targets RRM2) and panobinostat (targets ASF1B) in order to generate a synergistic effect on the inhibition of the tumor cell growth.

Furthermore, clinically used first-line chemotherapy drugs (e.g., a tyrosine kinase inhibitor, a mTOR inhibitor or a monoclonal antibody against VEGF) may also be combined with the compounds of the first aspect.

The present disclosure further provides the following itemized listing of embodiments:

Item 1. A compound or a pharmaceutically acceptable salt or prodrug thereof for use in a method of treatment of a renal cell carcinoma, such as clear cell renal cell carcinoma (ccRCC), wherein the compound is selected from the group consisting of TG-101209, panobinostat, NVP-TAE684 and withaferin-a.

Item 2. The compound for use according to item 1, wherein the compound is TG 101209.

Item 3. The compound for use according to item 2, wherein the method of treatment further comprises administration of panobinostat.

Item 4. The compound for use according to item 2 or 3, wherein the method of treatment further comprises administration of NVP-TAE684 or withaferin-a.

Item 5. The compound for use according to item 1, wherein the compound is panobinostat.

Item 6. The compound for use according to item 5, wherein the renal cell carcinoma is stage I-III ccRCC, preferably stage I or II ccRCC.

Item 7. The compound for use according to item 5 or 6, wherein the method of treatment further comprises administration of TG 101209.

Item 8. The compound for use according to any one of items 5-7, wherein the method of treatment further comprises administration of NVP-TAE684 or withaferin-a.

Item 9. The compound for use according to item 1, wherein the compound is NVP TAE684.

Item 10. The compound for use according to item 9, wherein the method of treatment further comprises administration of panobinostat.

Item 11. The compound for use according to item 9 or 10, wherein the method of treatment further comprises administration of TG-101209.

Item 12. The compound for use according to item 1, wherein the compound is withaferin-a.

Item 13. The compound for use according to item 12, wherein the method of treatment further comprises administration of panobinostat.

Item 14. The compound for use according to item 12 or 13, wherein the method of treatment further comprises administration of TG-101209.

Item 15. The compound for use according to any one of the preceding items, wherein the method of treatment further comprises administration of a drug selected from the group consisting of tyrosine kinase inhibitors (e.g. axitinib, sorafenib, pazopanib and sunitinib), mTOR inhibitors (e.g. everolimus and tesirolimus) and monoclonal antibodies against VEGF (e.g. bevacizumab).

Accordingly the compound TG-101209 or a pharmaceutically acceptable salt or prodrug thereof is provided for use in a method of treatment of a renal cell carcinoma, such as clear cell renal cell carcinoma (ccRCC). As explained above, TG-101209 targets BUB1B.

This method of treatment may further comprise administration of:
i) panobinostat (targeting ASF1B); and/or
ii) NVP-TAE684 or withaferin-a (targeting RRM2).

As indicated above, such a combination is expected to have a synergistic effect.

Further, the compound panobinostat or a pharmaceutically acceptable salt or prodrug thereof is provided for use in a method of treatment of a renal cell carcinoma, such as ccRCC. As explained above, panobinostat targets ASF1B.

This renal cell carcinoma is preferably stage I-III ccRCC, such as stage I or II ccRCC.

The method of treatment further comprises administration of:
i) TG-101209 (targeting BUB1B); and/or
ii) NVP-TAE684 or withaferin-a (targeting RRM2).

As indicated above, such a combination is expected to have a synergistic effect.

Also, the compound NVP-TAE684 or a pharmaceutically acceptable salt or prodrug thereof is provided for use in a method of treatment of a renal cell carcinoma, such as ccRCC. As explained above, NVP-TAE684 targets RRM2.

This method of treatment further comprises administration of:
i) panobinostat (targeting ASF1B); and/or
ii) TG-101209 (targeting BUB1B).

As indicated above, such a combination is expected to have a synergistic effect.

Finally, the compound withaferin-a or a pharmaceutically acceptable salt or prodrug thereof is provided for use in a method of treatment of a renal cell carcinoma, such as ccRCC. As explained above, withaferin-a targets RRM2.

This method of treatment further comprises administration of:
i) panobinostat (targeting ASF1B); and/or
ii) TG-101209 (targeting BUB1B).

As indicated above, such a combination is expected to have a synergistic effect.

The subject of the above-mentioned therapeutic methods is preferably a human.

The method of treatment may further comprise administration of a drug selected from the group consisting of tyrosine kinase inhibitors (e.g. axitinib, sorafenib, pazopanib and sunitinib), mTOR inhibitors (e.g. everolimus and tesirolimus) and monoclonal antibodies against VEGF (e.g. bevacizumab).

### Examples—Materials and Methods

### Data and Preprocessing

Transcript-expression profiles (TPM and count values) of 528 TCGA ccRCC samples and 72 adjacent normal samples were downloaded from https://osf.io/gqrz9[63]. The tumor and normal samples were extracted with sample and vial identifiers of BRC patient barcodes ‘01A’ and ‘11A’, respectively, which represent primary solid tumor tissue and solid normal tissue from the first vial, respectively. The mRNA expression was quantified by Kallisto [64] based on the GENCODE reference transcriptome (version 24) (Ensembl 83 (GRCh38.P5)). The clinical information of TCGA samples was downloaded by using the R package TCGAbiolinks [65]. The mRNA-seq data of 100 ccRCC samples of patients from the Japanese cohort [66] was downloaded from the European Genome-phenome Archive (Accession number: EGAS00001000509). BEDTools[67] was used to convert BAM to FASTQ file, and Kallisto was used for quantifying the count and TPM values of transcripts based on the same reference transcriptome of TCGA data. The sum value of the multiple transcripts of a gene was used as the expression value of this gene. The genes with average TPM values >1 were analyzed.

The essential scores of genes of 16 ccRCC cell lines were downloaded from DepMap Portal (https://depmap.org/portal/) [29], which are estimated based on the CRISPR-Cas9 essentiality screens. The meaning of the score is the essentiality of a gene for cancer cell survival after CRISPR-Cas9 knockout of this gene. More negative scores indicate more essential. The processed RNA-seq data of Caki-1 was downloaded from the Cancer Cell Line Encyclopedia (CCLE) portal (version: CCLE_RNAseq_rsem_transcripts_tpm_20180929.txt.gz) [68].

### Survival Analysis

Both the univariate Cox regression model and the Kaplan-Meier method were used to evaluate the association of gene expression with patients' overall survival (OS). For Cox analysis, an R package named ‘survival’ in which the input data is the expression values of each gene (TPM values) across all samples of patients, OS and survival outcomes of patients (death or alive) was used. For Kaplan-Meier analysis, the patients were classified into two groups based on the TPM values of each gene and examined their prognoses. Survival curves were estimated by the Kaplan-Meier method and compared by log-rank test. To choose the best TPM cutoffs for grouping the patients most significantly, all TPM values from the 20^{th} to 80^{th} percentiles used to group the patients, significant differences in the survival outcomes of the two groups were examined, and the value yielding the lowest log-rank P-value was selected.

### Functional Enrichment Analysis

The enrichGo function of the R package ClusterProfiler was used for Gene Ontology (GO) enrichment [69], which uses the hypergeometric distribution to estimate whether a list of genes is significantly enriched in each GO pathway. False discovery rate (FDR) was adjusted by the Benjamini-Hochberg (BH) method. FDR<0.05 was used to identify significantly enriched pathways.

### GCN Analysis and Gene Module Identification

Spearman correlation was used to estimate the correlation between each two genes across all tumor samples. The gene-gene links ranked in the top 1% with the highest correlation coefficients were extracted to construct the co-expression networks. A Random walks-based algorithm, Walktrap [22], was used to capture the gene modules with high transitivity from the co-expression network. The modules with more than 20 nodes and clustering coefficients higher than 0.6 were extracted for further analysis. R package ‘igraph’ was used for the topology analysis [70].

### Concordance Analysis of the Prognostic Genes

If two lists of prognostic genes, list 1 with L₁ genes and list 2 with L₂ genes, have k overlapping genes, among which s genes show the exact prognostic directions (both favorable or unfavorable) in the two gene lists, the probability of observing at least s consistent genes by chance can be estimated based on the following cumulative hypergeometric distribution model:

$$P = 1 - {\sum }_{i = 0}^{s - 1} \frac{( \begin{matrix}{L}_{2} \\ i\end{matrix} ) ( \begin{matrix}L - {L}_{2} \\ {L}_{1} - i\end{matrix} )}{( \begin{matrix}L \\ {L}_{1}\end{matrix} )}$$

L represents the number of the background genes commonly measured in the datasets from which the prognostic genes are extracted. The two gene lists were considered to be significantly overlapped if P<0.05. The concordance score s/k is used to represent the consistency between the two lists of genes. The score ranges from 0 to 1, and the higher concordance score indicates the better consistency of two lists of genes.

The hypergeometric distribution model was also used to test whether the favorable or unfavorable signatures significantly overlap with the genes involved in a functional module.

### Differential Expression Analysis

DESeq2 [71] was used to identify differentially expressed genes (DEGs) between TCGA tumor tissues and normal tissues groups. The lowly expressed genes with average TPM≤1 were removed, and the raw count values of the remaining genes were used as the input of DESeq2. FDR was adjusted by the BH method. FDR<0.05 was used to identify significant DEGs.

### Drug Repositioning

The shRNA- and drug-perturbed signature profiles (level 5 data) in HA1E, a kidney cell, were downloaded from the CMap data portal (https://clue.io/, version: CMAP LINCS 2020) [12]. Three or more biological replicates typically do the experiments in CMap. The level 5 data provides the replicate-collapsed Z-scores, representing a consensus biological response of transcriptomics to the perturbation of drug treatment or shRNA infection derived from different replicates[30]. Totally, 37,669 drug-perturbed signature profiles related to 6,986 drugs with different doses and time points and 21 shRNA-perturbed signature profiles related to BUB1B (six shRNAs), RRM2 (three shRNAs), CEP55 (three shRNAs), ASF1B (three shRNAs) and CCNB2 (six shRNAs) were obtained.

The drug repositioning approach hypothesizes that a drug is considered to have an inhibitory effect on the expression of a target gene if this drug leads to a wide perturbation on the gene expression landscape in tumor cells, similar to the effect of the knockdown of the target gene. Four procedures were applied to identify the drugs which had the highest possibility to inhibit the expression of their corresponding target genes by an integrated analysis of the shRNA- and drug-perturbed signature profiles. 1) Constructing the drug-shRNA matrix for each target gene. For a given target gene, the shRNA-perturbed signature profiles in HA1E cell line in which the Z-scores represent the biological dysregulation of gene expression in cells after shRNA infection was extracted. As one gene was knocked down by at least three shRNAs in the experiment setting in CMap, the shRNA-perturbed signature profiles were presented as a gene-shRNA matrix. Also, the drug-perturbed signature profiles in HA1E cell line in which the Z-scores represent the biological dysregulation of gene expression in cells after drug treatment were extracted and a gene-drug matrix was thus generated. Then, the Spearman correlation between each two possible lists of drug-perturbed and shRNA-perturbed signatures was calculated and a correlation coefficient matrix, named drug-shRNA matrix, in which each row represents a drug treatment by a specific dose and time point, and each column represents a specific shRNA for the knockdown of this target gene was thus generated. A correlation coefficient in the drug-shRNA matrix represents the similarity of effects on gene expression between specific drug treatment and specific shRNA knockdown. 2) Optimizing the drug-shRNA matrix. Since the drug treatment with different doses and time points could induce different effects on gene expression in cells as well as different similarities with the effects induced by shRNA knockdown, only the optimal dose and time point for each drug whose perturbation showed the highest similarity (correlation coefficient) with shRNA infection was extracted. Thus, the rows were simplified by keeping a unique and optimal dose and time point for each drug in the drug-shRNA matrix. A cell line may respond differently to different shRNAs even though they target the same genes. Thus, the column was simplified by extracting the optimal shRNAs whose signatures consistently correlate with drug perturbed signatures in the drug-shRNA matrix. A clustering analysis of different shRNAs was performed based on the correlation of different columns in the drug-shRNA matrix and the shRNAs, which were clustered together and showed better similarity with drug-perturbed effects, were extracted. Finally, three shRNAs for BUBIB, two shRNAs for RRM2, two shRNAs for CEP55, two shRNAs for ASF1B, and four shRNAs for CCNB2 were extracted. 3) Extracting the top 1% drug candidates. Based on the optimized drug-shRNA matrix, the drugs associated with each shRNA based on the correlation coefficient were ranked from the largest to smallest. A higher rank indicated a higher correlation. A list of drugs involved in the top 1% with the highest ranks associated with each shRNA was first extracted, and then the overlapped drugs among the top 1% lists related to different shRNAs were selected as candidates. 4) Selecting the three most effective drugs for each target. The top three drugs with the highest median ranks were finally considered the most effective drugs for each target.

The drug repositioning for BUB1B was taken as an example. The drug-shRNA matrix of BUB1B included 37,669 rows associated with 6,986 drugs with different doses and time points and six columns related to six different shRNAs. After optimization, a simplified drug-matrix with 6,986 drugs, each with the best dose and time point and three optimal shRNAs for knockdown of BUB1B, was obtained. Then, the top 1% drugs (70 drugs) with the highest correlation coefficients associated with each shRNA were extracted. 24 overlapped drugs were found among the three lists of top 1% drugs. Among these drugs, the top three drugs, TG-101209, oxetane, and WH-4-025, with the highest median ranks, were finally selected as the most effective drugs for targeting BUB1B.

### IHC Image

The IHC images of ccRCC tumor tissues and normal kidney tissues were downloaded from the Human Protein Atlas website (https://www.proteinatlas.org/)[27,28]. The protocol of IHC was provided in [27,28]. For a given gene, the IHC images of normal tissue and tumor tissue conducted by the same antibody were selected. TPX2, ASF1B, CCNB2 and TCF4 IHC images were done by the HPA005487, HPA069385, HPA008873 and HPA020722 antibodies, respectively.

### Cell Culture

Cell culture 1: Human clear cell renal cell carcinoma (ccRCC) cell line Caki-1 was obtained from the Karolinska Institute of Environmental Medicine, Stockholm, Sweden, derived from a male ccRCC patient. The cells were cultured in RPMI 1640 medium with 10% fetal bovine serum (FBS) and 1% penicillin/streptomycin. The cells were cultivated at 37° C. in a humidified incubator with 5% CO₂.

Cell culture 2: Caki-1 was purchased from CLS Cell Lines Service (Germany). The cells were maintained in McCoy's SA (modified) medium (16600082, Thermo Fisher Scientific, USA) and supplemented with 10% fetal bovine serum (FBS, F7524, Sigma-Aldrich), 1% Penicillin/Streptomycin (P4333, Sigma-Aldrich).

siRNA Transfection

20×10³ cells (from cell culture 1) were plated by quadruplicate into a 96-well plate per well. Day after seeding, 1 pmol siRNAs for targeting BUB1B, RRM2, ASF1B, CCNB2 and CEP55 (OriGene Technologies Inc., USA) were transfected into cells using the Lipofectamine® RNAiMAX reagent (Invitrogen) for three days. Then, the cells were used for cell viability assay and protein lysate was harvested for Western blot analysis after 3-day transfection.

### Drug Treatment

TG-101209, NVP-TAE684, MK-0752, actinomycin-d, and panobinostat were purchased from Selleckchem (S2692, S1108, S2660, S8964, S1030, Selleckchem, Houston, TX, USA), and withaferin-a was purchased from Sigma (W4394, Sigma-Aldrich, Saint Louis, MO, USA). These drugs were dissolved in DMSO. The cells (from cell culture 1) were seeded in a 96-well plate at 20×10³ cells per well. After seeding for 24 h, the cells were treated for 24 h with the target drugs: TG-101209 (6 nM), NVP-TAE684 (3 nM), MK-0752 (5 nM), withaferin-a (4 uM), actinomycin-d (0.3 nM), and panobinostat (5 nM). The cells in the negative control group were only treated by DMSO for one day.

The target drugs developed as alternatives to TG-101209, were dissolved in 0.1% DMSO (41639, Sigma-Aldrich). Caki-1 cells from cell culture 2 were seeded in a 6-well plate at 3×10⁵ cells per well. After seeding for 24 h, the cells were treated with 5 μM of the respective target drugs for 2 days.

### Western Blot Analysis

Cells Treated with TG-101209, NVP-TAE684, MK-0752, Actinomycin-d, and Panobinostat

The cells were washed with phosphate-buffered saline (PBS) and lysed with CelLytic M (C2978, Sigma-Aldrich, Saint Louis, MO, USA) lysis buffer containing protease inhibitors. The lysates were centrifuged at 12 000 rpm for 5 min, and the supernatant was collected. The proteins were separated by Mini-PROTEAN® TGX™ Precast Gels (BioRad, Berkeley, CA, USA) and transferred to a Trans-Blot Turbo Mini 0.2 um PVDF Transfer Packs membrane (BioRad, Berkeley, CA, USA) by using Trans-Blot® Turbo™ Transfer System (Bio-Rad, Berkeley, CA, USA). The antibodies for BUB1B (HPA008419), RRM2 (HPA056994), CEP55 (HPA023430), ASF1B(HPA069385), CCNB2 (HPA008873), and GAPDH (sc47724, Santa Cruz Biotechnology, Inc.) were used for primary immunoblotting. All the antibodies were diluted at 1:10000 concentration. The membranes were incubated in primary antibody solution overnight at 4° C. with gentle rocking. Secondary antibody, goat Anti-Rabbit HRP (ab205718) or goat anti-mouse IgG-HRP (sc2005, Santa Cruz Biotechnology, Inc.), was blotted for 30 min at 4° C. with gentle rocking. The protein bands were detected with ImageQuanattm LAS 500 (29-0050-63, GE) automatic exposure procedure or 6 min exposure.

Cells Treated with Synthesized TG-101209 Alternatives

The cells were washed with PBS solution and then lysed with CelLytic M (C2978, Sigma-Aldrich) lysis buffer containing protease inhibitors (11836170001, Roche). The cell lysates were centrifuged at 12 000 rpm for 10 min and supernatants were collected. Protein Assay Dye Reagent (5000006, Bio-Rad) was used for the determination of protein concentration. The absorbance of the proteins was measured spectrophotometrically at 595 nm by a microplate reader (Hidex Sense Beta Plus). Protein electrophoresis was performed using Mini-PROTEAN® TGX™ Precast Gels (4561086, Bio-Rad) and then the separated proteins were transferred to a Trans-Blot Turbo Mini 0.2 um PVDF Transfer Packs membrane (1704158, Bio-Rad) by using Trans-Blot® Turbo™ Transfer System (Bio-Rad). The membrane was blocked with 5% skim milk for 30 min at 4° C. with gentle rocking. After blocking, the membrane was treated with primary antibodies: Anti-BUB1B (HPA008419, Human Protein Atlas, Sweden) and Anti-Beta actin (ab8227, Abcam) as a loading control overnight at 4° C. on a rocking platform. After the treatment of primary antibodies, the membrane was washed three times with TBS-T buffer (A09-7500-100, Medicago). Then, Goat anti-Rabbit IgG-HRP (ab205718, Abcam) was treated as the secondary antibody for 30 min at 4° C. with gentle rocking. The protein bands on the membrane were revealed using enhanced chemiluminescence substrate (WBLUF0500, Merck) and detected with ImageQuant™ LAS 500 (GE Healthcare).

### Cell Viability Assay

Cell Viability Assay for siRNA Transfected Cells and TG-101209, NVP-TAE684. MK-0752. Actinomycin-d. And Panobinostat Treated Cells

Cell proliferation was detected by Cell Counting Kit-8 (CCK-8) assay. The 10 μl of CCK-8 reagent (1:10) was added to each well of 96-well plate with siRNA transfected cells or drug-treated cells by manufacturer's instruction. The 96-well plate was incubated at 37° C. for 2 hours and then measured absorbance at 450 nm using a microplate reader (Hidex Sense Beta Plus).

Cell Viability Assay for Cells Treated with TG-101209 Alternatives

The cytotoxic effects of the TG-101209 and its alternatives on the Caki-1 cells were tested by MTT (Thiazolyl Blue Tetrazolium Bromide) method. 1.5×104 cells were seeded per well in a 96-well plate in 100 μL growth media. After 1 day of cell seeding, 5 μM of the target drugs were dissolved in 0.1% DMSO and 100 μL of the media was treated for one day. After the incubation period, 5 mg/ml MTT (M2128, Sigma-Aldrich) solution in PBS (10 μL) was added to each well. After 1 hour, the MTT solution and all media were removed from the wells. 100 μl of DMSO was added and mixed to dissolve the formazan crystals. Cell viability was analysed by measuring the absorbance of the dissolved formazan at a wavelength of 570 nm with a microplate reader (Hidex Sense Beta Plus).

### LDH Assay

Lactate dehydrogenase (LDH) assay was performed with LDH Assay Kit (ab65393). 1.5×104 cells per well (from cell culture 2) were seeded in a 96-well plate and drugs were treated at 10 μM for 1 day with 200 μl media. 20 μl of media was analysed with LDH Assay Kit as per manufacturer's instruction.

### Synthesis of TG-101209 Alternatives

### Materials and Instrumentation

All reagents were purchased from commercial suppliers and were used without further purification. Reactions were monitored by LC-MS (Thermo Fisher TSQ Series, Athena C18-WP, 100 Å, 2.1×50 mm, 3 μm); Water:MeOH (0.01 formic acid)) or by Thin-Layer Chromatography (TLC) using silica gel pre-coated aluminum plates (Kieselgel 60, 254, E. Merck, Germany). The chromatograms were visualized using ultraviolet light (254 and 366 nm). 1H NMR spectra were recorded on Avance Bruker 500 MHz spectrometer in CDCl₃ and DMSO-d6. ¹³C NMR spectra were recorded in deuterated solvents on Bruker spectrometer at 126 MHz, with the central peak of the deuterated solvent as the internal standard. The 1H NMR spectra are reported as S/ppm downfield from tetramethyl silane as the internal standard. The following abbreviations are used for the proton spectra multiplicities: s, singlet; d, doublet; t, triplet; q, quartet; qt, quintuplet; and m, multiplet. Coupling constants (J) are reported in Hertz (Hz). Flash-column chromatography was performed on PuriFlash XS 520 Plus Flash chromatography system (Interchim) with built-in UV-detector, ELSD-detector and fraction collector with Interchim silica gel columns.

### Synthesis of (S)—N-(1-(4-bromophenyl)ethyl)-3-((5-methyl-2-((4-(4-methylpiperazin-1-yl)phenyl)amino)pyrimidin-4-yl)amino)benzenesulfonamide

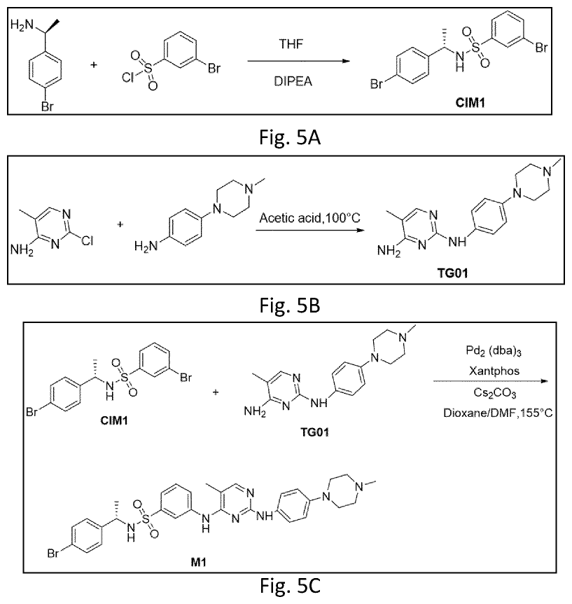

Step 1: N, N-diisopropylethylamine (0.7 mL, 3.9 mmol) and (S)-1-(4-bromophenyl)ethanamine (0.6 mL, 4.0 mmol) were added to a solution of 3-bromobenzenesulfonyl chloride (1.0 g, 3.9 mmol), in tetrahydrofuran (15 mL). The resulting mixture was stirred at room temperature. After completion of the reaction, the solvent was removed under reduced pressure. Water (15 mL) was added, and the resulting aqueous phase was extracted with CH₂Cl₂ (3×15 mL). The organic layer was dried over anhydrous sodium sulphate and concentrated to give (S)-3-bromo-N-(1-(4-bromophenyl)ethyl)benzenesulfonamide (CIM1) (1.4 g, yield: 85%) as a white solid (see FIG. 5A). MS (ESI): m/z 420.0 [M+H]+.

Step 2: A mixture of 2-chloro-5-methylpyrimidin-4-amine (1.0 g, 7.0 mmol) and 4-(4-methylpiperazin-1-yl)phenylamine (1.35 g, 7.0 mmol) in acetic acid (15 mL) was heated at 100° C. for 3 h. The mixture was allowed to cool to room temperature and the acetic acid was removed under reduced pressure. The residue was added to water (20 mL) and the mixture was neutralized with 10% NaOH solution until precipitation occurred. After filtration and washing with water, the intermediate 5-methyl-N2-(4-(4-methylpiperazin-1-yl)phenyl)pyrimidine-2,4-diamine (TG01) was obtained as a grey solid (1.4 g, 67%) (see FIG. 5B). It was used in the next step without further purification.

Step 3: A mixture of TG01 (0.1 g, 0.3 mmol), CIM1 (0.18 g, 0.36 mmol), Pd₂(dba)₃ (0.03 g, 0.03 mmol), Xantphos (0.03 g, 0.045 mmol) and cesium carbonate (0.22 g, 0.6 mmol) was suspended in dioxane/DMF (3:1, 8 mL) and heated at 155° C. under a nitrogen atmosphere for 24 hours. After completion of the reaction, the reaction mixture was cooled to room temperature and diluted with DCM (10 mL). The mixture was filtered, and the filtrate was concentrated under vacuum. The residue was purified by column chromatography (chloroform:methanol=9:1) to give the desired compound (M1) (40 mg, yield: 19%) as a white solid (see FIG. 5C). MS (ESI): m/z 637. 14 [M+H]+.

### Synthesis of 3-((5-methyl-2-((4-(4-methylpiperazin-1-yl)phenyl)amino)pyrimidin-4-yl)amino)-N-phenylbenzenesulfonamide (M3)

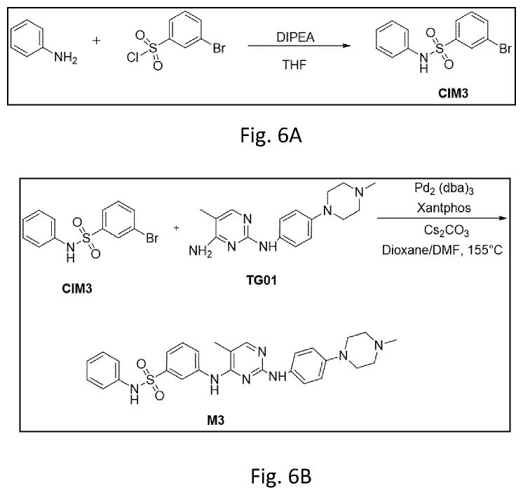

Step 1: N, N-diisopropylethylamine (0.7 mL, 3.9 mmol) and aniline (0.36 mL, 3.95 mmol) were added to a solution of 3-bromobenzenesulfonyl chloride (1.0 g, 3.9 mmol) in Tetrahydrofuran (15 mL). The resulting mixture was stirred at room temperature. After completion of the reaction, the solvent was removed under reduced pressure. Water (15 mL) was added to the reaction mixture, and the resulting aqueous phase was extracted with CH₂Cl₂ (3×20 mL). The organic layer was dried over anhydrous sodium sulphate and concentrated to give 3-bromo-N-phenylbenzenesulfonamide (CIM3) (1.1 g, yield: 90%) as a white solid (see FIG. 6A). MS (ESI): m/z 313.12 [M+H]+.

Step 2: A mixture of intermediate TG01 (0.1 g, 0.3 mmol), CIM3 (0.11 g, 0.36 mmol), Pd₂(dba)₃ (0.02 g, 0.02 mmol), Xantphos (0.03 g, 0.045 mmol) and cesium carbonate (0.2 g, 0.6 mmol) was suspended in dioxane/DMF (3:1, 8 mL) and heated at 155° C. under the nitrogen atmosphere for 24 hours, after completion of the reaction, the reaction mixture was cooled to room temperature and diluted with DCM (10 mL). The mixture was filtered and the filtrate was concentrated under vacuum. The residue was purified by column chromatography (chloroform:methanol=9:1) to give the desired compound (M3) (55 mg, yield: 30%) as a white solid (see FIG. 6B). MS (ESI): m/z 530. 23 [M+H]+.

¹H NMR (500 MHz, DMSO-d₆) δ 10.32 (s, 1H), 8.69 (s, 1H), 8.53 (s, 1H), 8.19 (d, J=8.1 Hz, 1H), 7.88 (s, 1H), 7.47-7.38 (m, 5H), 7.21 (dd, J=7.4, 8.5 Hz, 2H), 7.11 (dd, J=1.1, 8.6 Hz, 2H), 7.00 (t, J=7.9 Hz, 1H), 6.80 (d, J=9.1 Hz, 2H), 3.03-3.01 (m, 4H), 2.46-2.43 (m, 4H), 2.22 (s, 3H), 2.09 (s, 3H).

### Synthesis of N-cyclobutyl-3-((5-methyl-2-((4-(4-methylpiperazin-1-yl)phenyl)amino)pyrimidin-4-yl)amino)benzenesulfonamide (M5)

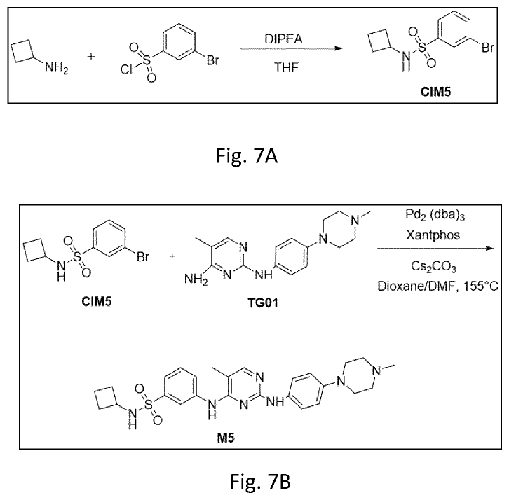

Step 1: N, N-diisopropylethylamine (0.7 mL, 3.9 mmol) and cyclobutanamine (0.3 mL, 4.0 mmol) were added to a solution of 3-bromobenzenesulfonyl chloride (1.0 g, 3.9 mmol) in tetrahydrofuran (15 mL. The resulting mixture was stirred at room temperature. After completion of the reaction, the solvent was removed under reduced pressure. Water (15 mL) was added to the reaction mixture and the resulting aqueous phase was extracted with CH₂Cl₂ (3×20 mL). The organic layer was dried over anhydrous sodium sulphate and concentrated to give 3-bromo-N-cyclobutylbenzenesulfonamide (CIM5) (0.8 g, yield: 70%) as a white solid (see FIG. 7A). MS (ESI): m/z 291.89 [M+H]+.

Step 2: A mixture of intermediate TG01 (0.12 g, 0.4 mmol), CIM5 (0.14 g, 0.48 mmol), Pd₂(dba)₃ (0.035 g, 0.04 mmol), Xantphos (0.035 g, 0.06 mmol) and cesium carbonate (0.25 g, 0.8 mmol) was suspended in dioxane/DMF (3:1, 8 mL) and heated at 155° C. under the nitrogen atmosphere for 20 hours. After completion of the reaction, the reaction mixture was cooled to room temperature and diluted with DCM (10 mL). The mixture was filtered and the filtrate was concentrated under vacuum. The residue was purified by column chromatography (chloroform:methanol=9:1) to give the desired compound (M5) (80 mg, yield: 40%) as a light yellow solid (see FIG. 7B). MS (ESI): m/z 508. 17 [M+H]+.

¹H NMR (500 MHz, DMSO-d₆) δ 8.71 (s, 1H), 8.53 (s, 1H), 8.21 (d, J=7.9 Hz, 1H), 7.98-7.93 (m, 2H), 7.89 (s, 1H), 7.49-7.43 (m, 4H), 6.79 (d, J=9.0 Hz, 2H), 3.63 (m, 1H), 3.03-3.00 (m, 4H), 2.46-2.44 (m, 4H), 2.22 (s, 3H), 2.11 (s, 3H), 1.92 (m, 2H), 1.80-1.74 (m, 2H), 1.49-1.41 (m, 2H).

### Synthesis of 5-methyl-N2-(4-(4-methylpiperazin-1-yl)phenyl)-N4-(3-(morpholinosulfonyl)phenyl)pyrimidine-2,4-diamine (M7)

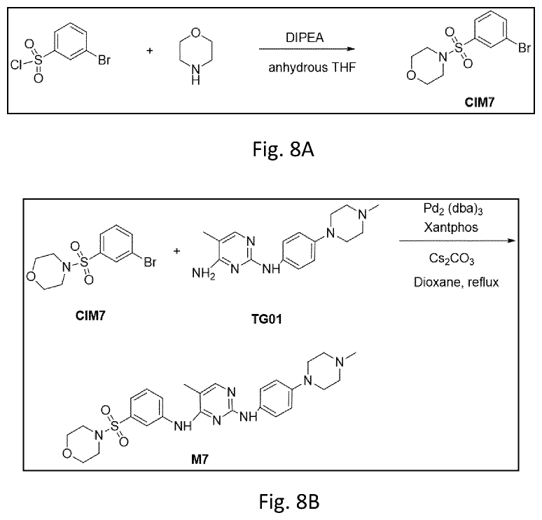

Step 1: N, N-diisopropylethylamine (2.25 mL, 12.7 mmol) and morpholine (1.1 mL, 12.7 mmol) were added to a solution of 3-bromobenzenesulfonyl chloride (3.1 g, 12.1 mmol) in tetrahydrofuran (20 mL). The resulting mixture was stirred at room temperature. After completion of the reaction, the solvent was removed under reduced pressure and water (15 mL) was added. The resulting aqueous phase was extracted with CH₂Cl₂ (3×20 mL). The organic layer was dried over anhydrous sodium sulphate and concentrated to give 4-((3-bromophenyl)sulfonyl)morpholine (CIM7) (3.4 g, yield: 90%) as a white solid (see FIG. 8A). MS (ESI): m/z 307.89[M+H]+.

Step 2: A mixture of intermediate TG01 (0.1 g, 0.3 mmol), 4-((3-bromophenyl)sulfonyl)morpholine (CIM7) (0.12 g, 0.36 mmol), Pd2(dba)3 (0.03 g, 0.03 mmol), Xantphos (0.04 g, 0.06 mmol) and cesium carbonate (0.25 g, 0.8 mmol) was suspended in dioxane (6 mL) and heated at 105° C. under the nitrogen atmosphere for 16 hours, after completion of the reaction, the reaction mixture was cooled to room temperature and diluted with DCM (10 mL). The mixture was filtered and the filtrate concentrated under vacuum. The residue was purified by column chromatography (chloroform:methanol=9:1) to give the desired compound (M7) (90 mg, yield: 51%) as a white solid (see FIG. 8B). MS (ESI): m/z 524. 26 [M+H]+.

### Synthesis of 5-methyl-N4-(5-methyl-4-(4-(trifluoromethyl)phenyl)pyrimidin-2-yl)-N2-(4-(4-methylpiperazin-1-yl)phenyl)pyrimidine-2,4-diamine (M94)

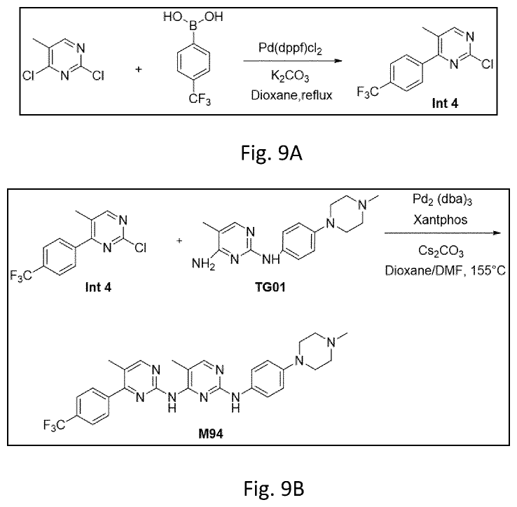

Step 1: A mixture of 2,4-dichloro-5-methylpyrimidine (0.3 g, 1.8 mmol), (4-(trifluoromethyl)phenyl) boronic acid (0.38 g, 1.98 mmol), K₂CO₃ (0.75 g, 5.4 mmol) PdCl₂(dppf) (0.13 g, 0.18 mmol), and H2O (15 μL) was suspended in dioxane (5 mL) and heated with stirring at 105° C. under a nitrogen atmosphere for 10 hours. After completion of the reaction, the solvent was removed and the crude material was purified by silica gel chromatography (0 to 10% EtOAc gradient in hexanes) to obtain (157 mg, yield: 31%) of Int 4 (see FIG. 9A) as a white powder.

Step 2: A mixture of intermediate TG01 (0.15 g, 0.48 mmol), Int 4 (0.12 g, 0.44 mmol), Pd₂(dba)₃ (0.04 g, 0.048 mmol), Xantphos (0.08 g, 0.13 mmol) and cesium carbonate (0.3 g, 0.9 mmol) was suspended in dioxane/DMF (3:1.5 mL) and heated at 120° C. under a nitrogen atmosphere for 20 hours. After completion of the reaction, the reaction mixture was cooled to room temperature and diluted with CHCl₃ (15 mL). The mixture was filtered and the filtrate was concentrated under vacuum. The residue was purified by column chromatography (chloroform:methanol=9:1) to give the desired compound M94 (35 mg, yield: 15%) as a yellow solid (see FIG. 9B). MS (ESI): m/z 535. 26 [M+H]+.

¹H NMR (500 MHz, DMSO-d₆) δ 9.33 (s, 1H), 8.86 (s, 1H), 8.63 (s, 1H), 8.04 (s, 1H), 7.93 (d, J=8.2 Hz, 2H), 7.84 (d, J=8.4 Hz, 2H), 7.50 (d, J=9.0 Hz, 2H), 6.48 (d, J=8.9 Hz, 2H), 2.91-2.89 (m, 4H), 2.42-2.39 (m, 4H), 2.31 (s, 3H), 2.19 (s, 3H), 2.09 (s, 3H).

### Synthesis of N-(tert-butyl)-3-((2-((6-(4-chlorophenoxy)pyridin-3-yl)amino)-5-methylpyrimidin-4-yl)amino)benzenesulfonamide (M24)

Step 1: N, N-diisopropylethylamine (2.88 mL, 16.43 mmol) and tert-butylamine (1.73 mL, 16.43 mmol) were added to a solution of 3-bromobenzenesulfonyl chloride (4 g, 15.65 mmol) in tetrahydrofuran (20 mL). The resulting mixture was stirred at room temperature. After completion of the reaction, the solvent was removed under reduced pressure and water (15 mL) was added. The resulting aqueous phase was extracted with CH₂Cl₂ (3×20 mL). The organic layer was dried over anhydrous sodium sulphate and concentrated to give 3-bromo-N-(tert-butyl)benzenesulfonamide (A, 4.1 g, yield: 90%) as a white solid (see FIG. 9A). MS (ESI): m/z 293.0 [M+H]+.

Step 2: To a solution of 3-bromo-N-(tert butyl) benzenesulfonamide (1.96 g, 6.7 mmol) and 2-chloro-5-methylpyrimidin-4-amine (0.8 g, 5.6 mmol) in 1,4-dioxane (15 mL) was added cesium carbonate (3.64 g, 11.2 mmol), tri(dibenzylacetone)diphenyl palladium (0.26 g, 0.28 mmol) and 4,5-diphenylphosphine-9,9-dimethoxyanthracene (0.48 g, 0.84 mmol). The mixture was stirred at 105° C. for 6 hours under N₂ atmosphere. Then, the reaction mixture was cooled to room temperature and diluted with chloroform (20 mL). The mixture was filtered and the filtrate was concentrated under vacuum. The residue was dissolved in EtOAc and hexane was added until precipitation occurred. After filtration, N-(tert-butyl)-3-((2-chloro-5-methylpyrimidin-4-yl)amino)benzenesulfonamide (TGo) (1.6 g, 82%) was obtained as a yellow solid (see FIG. 9B). MS (ESI): m/z 355. 04 and 357.03 [M+H]+. It was used in the next step without purification.

Step 3: A mixture of intermediate TGo (0.1 g, 0.28 mmol), 6-(4-chlorophenoxy)pyridin-3-amine CIM24 (0.065 g, 0.29 mmol), Pd2(dba)3 (0.018 g, 0.014 mmol), Xantphos (0.024 g, 0.042 mmol) and cesium carbonate (0.18 g, 0.56 mmol) was suspended in dioxane (5 mL) and heated at reflux under a nitrogen atmosphere for 18 hours. The reaction mixture was cooled to room temperature and diluted with DCM (10 mL). The mixture was filtered and the filtrate was concentrated under vacuum. The residue was purified by column chromatography (hexane:ethyl acetate=7:3) to give N-(tert-butyl)-3-((2-((6-(4-chlorophenoxy)pyridin-3-yl)amino)-5-methylpyrimidin-4-yl)amino)benzenesulfonamide (M24) (20 mg, yield: 13%) as a white solid (see FIG. 9C). MS (ESI): m/z 537.16 and 538.16 [M−H]−.

### Synthesis of N-(tert-butyl)-3-((5-methyl-2-((3-(morpholinosulfonyl)phenyl)amino)pyrimidin-4-yl)amino)benzenesulfonamide (M25)

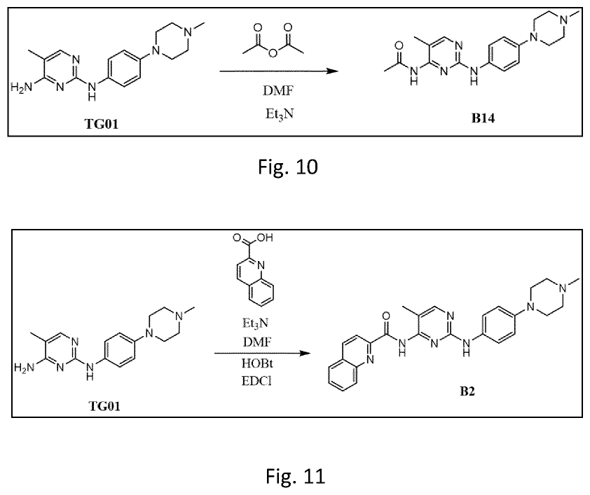

A mixture of intermediate TGo (0.1 g, 0.28 mmol), 3-(morpholinosulfonyl)aniline CIM25 (0.075 g, 0.3 mmol), Pd₂(dba)₃ (0.018 g, 0.014 mmol), Xantphos (0.024 g, 0.042 mmol) and cesium carbonate (0.18 g, 0.56 mmol) was suspended in dioxane (5 mL) and heated at reflux under a nitrogen atmosphere for 20 hours. The reaction mixture was cooled to room temperature and diluted with DCM (10 mL). The mixture was filtered and the filtrate was concentrated under vacuum. The residue was purified by column chromatography (hexane:ethyl acetate=7:3) to give N-(tert-butyl)-3-((5-methyl-2-((3-(morpholinosulfonyl)phenyl)amino)pyrimidin-4-yl)amino)benzenesulfonamide (M25) (24 mg, yield: 15%) as a yellow solid (see FIG. 10). MS (ESI): m/z 561. 19 [M+H]+.

### Synthesis of N-(tert-butyl)-3-((5-methyl-2-((4-(N-(5-methylisoxazol-3-yl)sulfamoyl)phenyl)amino)pyrimidin-4-yl)amino)benzenesulfonamide (M47)

A mixture of intermediate TGo (0.1 g, 0.28 mmol), sulfamethoxazole (0.075 g, 0.29 mmol), Pd₂(dba)₃ (0.018 g, 0.014 mmol), Xantphos (0.024 g, 0.042 mmol) and cesium carbonate (0.18 g, 0.56 mmol) was suspended in dioxane/DMF (10:3, 6.5 mL), then heated at 120° C. under a nitrogen atmosphere for 18 hours. After cooling to room temperature, the resulting mixture was filtered and the filtered solid was washed with DCM. The filtrate was concentrated. The residue was purified by column chromatography (hexane:ethyl acetate=6:4, with a few drops of methanol) to give N-(tert-butyl)-3-((5-methyl-2-((4-(N-(5-methylisoxazol-3-yl)sulfamoyl)phenyl)amino)pyrimidin-4-yl)amino)benzenesulfonamide (M47) (40 mg, yield: 25%) as a yellow solid (see FIG. 11). MS (ESI): m/z 572. 17 [M+H]+.

¹H NMR (500 MHz, DMSO) δ 11.17 (s, 1H), 9.57 (s, 1H), 8.73 (s, 1H), 8.07 (s, 1H), 8.05 (s, 1H), 8.01 (s, 1H), 7.81 (d, J=8.9 Hz, 2H), 7.63-7.50 (m, 5H), 6.13 (s, 1H), 2.29 (s, 3H), 2.15 (s, 3H), 1.12 (s, 9H).

### Synthesis of 3-((2-((3-((6,7-bis(2-methoxyethoxy)quinazolin-4-yl)amino)phenyl)ethynyl)-5-methylpyrimidin-4-yl)amino)—N-(tert-butyl)benzenesulfonamide (M48)

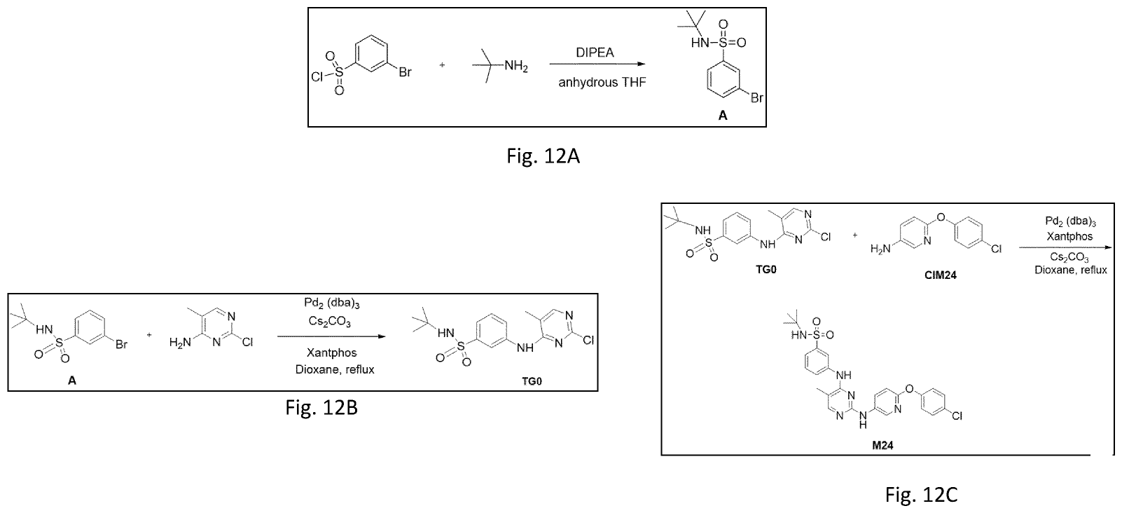

A mixture of TGo (0.11 g, 0.3 mmol), erlotinib (0.1 g, 0.25 mmol), PdCl₂(PPh₃)₂ (10 mol %, 0.02 g), CuI (5 mol %, 0.002 g) and K3PO4 (0.07 g, 0.32 mmol) in anhydrous DMF (4 mL) was stirred vigorously at 90° C. for 24 h under a nitrogen atmosphere. After completion of the reaction, the resulting mixture was quenched by water (30 mL) and extracted with chloroform (3×20 mL). The combined organic layers were washed with water, dried over anhydrous MgSO₄, and concentrated under reduced pressure. The residue was purified by column chromatography on silica gel using hexane/isopropanol (5:5) as eluent to afford 3-((2-((3-((6,7-bis(2-methoxyethoxy)quinazolin-4-yl)amino)phenyl)ethynyl)-5-methylpyrimidin-4-yl)amino)-N-(tert-butyl)benzenesulfonamide (M48) as a white solid (10 mg, yield: 5%) (see FIG. 12). MS (ESI): m/z 712. 28 [M+H]+.

### Synthesis of 3,3′-((5-methylpyrimidine-2,4-diyl)bis((azanediyl))bis(N-(tert-butyl)benzenesulfonamide) (M55)

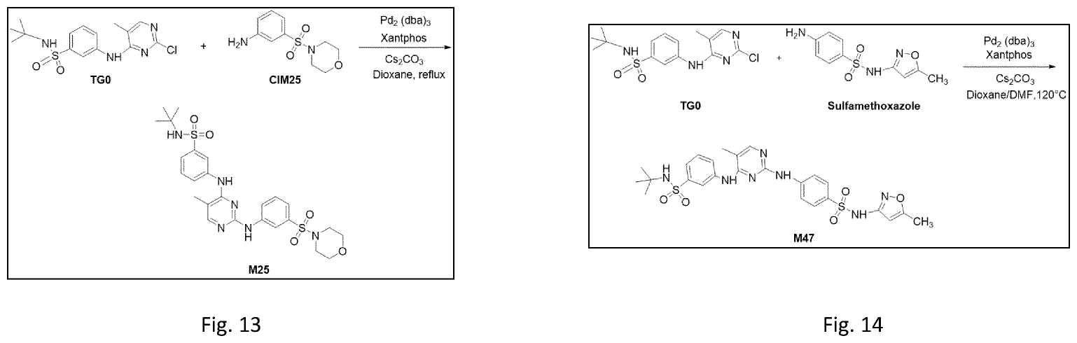

A mixture of TGo (0.2 g, 0.57 mmol), N-tert-Butyl 3-Aminophenylsulfonamide CIM55 (0.12 g, 0.52 mmol), Cs2CO3 (0.34 g, 1.04 mmol), Pd2(dba)3 (0.05 g, 0.052 mmol), Xantphos (0.06 g, 0.1 mmol) in Dioxane (5 mL) was prepared under an atmosphere of nitrogen. The resulting mixture was flushed with nitrogen and heated to 105° C. for 20 h. After completion of the reaction, the mixture was then allowed to cool to room temperature and diluted with CHCl3 (10 mL). The mixture was filtered and the filtrate concentrated under vacuum.. The residue was purified by column chromatography (hexane:ethyl acetate=6:4) to give 3,3′-((5-methylpyrimidine-2,4-diyl)bis(azanediyl))bis(N-(tert-butyl)benzenesulfonamide) (M55) (60 mg, yield: 21%) as a white solid (see FIG. 13). MS (ESI): m/z 547. 23 [M+H]+.

¹H NMR (500 MHz, DMSO) δ 9.31 (s, 1H), 8.63 (s, 1H), 8.21 (dd, J=1.8, 7.0 Hz, 1H), 8.16 (s, 1H), 8.12 (s, 1H), 7.99 (d, J=3.8 Hz, 2H), 7.51 (s, 1H), 7.50 (s, 1H), 7.42 (s, 1H), 7.38-7.29 (m, 3H), 2.16 (s, 3H), 1.12 (s, 9H), 1.11 (s, 9H).

### Synthesis of 5-methyl-N2,N4-bis(3-(morpholinosulfonyl)phenyl)pyrimidine-2,4-diamine (M57)

To a solution of 4-((3-bromophenyl)sulfonyl)morpholine (CIM7) (0.77 g, 2.5 mmol) and 2-chloro-5-methylpyrimidin-4-amine (0.3 g, 2.1 mmol) in 1,4-dioxane (10 mL) was added cesium carbonate (1.36 g, 4.2 mmol), tri(dibenzylacetone)diphenyl palladium (0.1 g, 0.1 mmol) and 4,5-diphenylphosphine-9,9-dimethoxyanthracene (0.18 g, 0.3 mmol). The mixture was stirred at 105° C. for 5 hours under N₂ atmosphere. Then, the reaction mixture was cooled to room temperature and diluted with CHCl₃ (15 mL). The mixture was filtered and the filtrate was concentrated in vacuo. The residue was dissolved in EtOAc and hexane was added until solid precipitation occurred. After filtration, 2-chloro-5-methyl-N-(3-(morpholinosulfonyl)phenyl)pyrimidin-4-amine (SM-M7) (0.7 g, 90%) was obtained as a yellow solid (see FIG. 14A). It was used in the next step without purification.

A mixture of intermediate SM-M7 (0.12 g, 0.32 mmol), 3-(morpholinosulfonyl)aniline CIM25 (0.09 g, 0.35 mmol), Pd₂(dba)₃ (0.02 g, 0.02 mmol), Xantphos (0.028 g, 0.048 mmol) and cesium carbonate (0.18 g, 0.64 mmol) was suspended in dioxane (5 mL) and heated at reflux under a nitrogen atmosphere for 18 hours. The reaction mixture was cooled to room temperature and diluted with CHCl3 (10 mL). The mixture was filtered and the filtrate was concentrated under vacuum. The residue was purified by column chromatography (hexane:ethyl acetate=5:5) to give 5-methyl-N2,N4-bis(3-(morpholinosulfonyl)phenyl)pyrimidine-2,4-diamine M57 (30 mg, yield: 16%) as a brock white (see FIG. 14B). MS (ESI): m/z 575. 14 [M+H]+.

¹H NMR (500 MHz, DMSO) δ 9.44 (s, 1H), 8.74 (s, 1H), 8.50 (d, J=8.1 Hz, 1H), 8.12 (s, 1H), 8.02 (s, 1H), 7.84 (s, 1H), 7.62 (t, J=8.0 Hz, 1H), 7.46 (t, J=8.0 Hz, 1H), 7.40-7.36 (m, 3H), 3.63 (m, 8H), 2.89 (m, 4H), 2.86 (m, 4H), 2.16 (s, 3H).

### Synthesis of N2-(6-(4-chlorophenoxy)pyridin-3-yl)-5-methyl-N4-(3-(morpholinosulfonyl) phenyl)pyrimidine-2,4-diamine (M58)

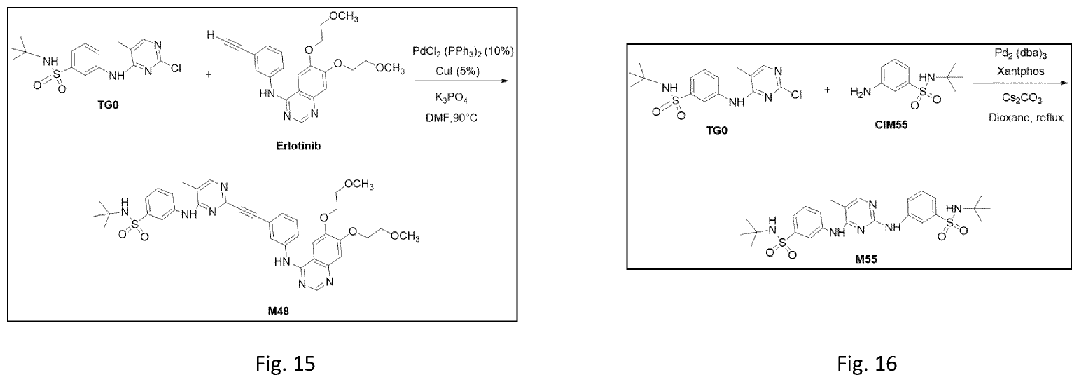

A mixture of intermediate SM-M7 (0.12 g, 0.32 mmol), 6-(4-chlorophenoxy)pyridin-3-amine (CIM24, 0.085 g, 0.35 mmol), Pd₂(dba)₃ (0.02 g, 0.02 mmol), Xantphos (0.028 g, 0.048 mmol) and potassium tert-butoxide (0.05 g, 0.48 mmol) was suspended in dioxane (5 mL) and heated at reflux under a nitrogen atmosphere for 20 hours. The reaction mixture was cooled to room temperature and diluted with DCM (10 mL). The mixture was filtered and the filtrate was concentrated under vacuum. The residue was purified by column chromatography (hexanes:ethyl acetate=6:4) to give the desired product M58 (22 mg, yield: 12%) as a light yellow solid (see FIG. 15). MS (ESI): m/z 554.18 [M+H]+.

¹H NMR (500 MHz, DMSO) δ 9.16 (s, 1H), 8.70 (s, 1H), 8.39-8.34 (m, 2H), 8.16 (dd, J=2.7, 8.9 Hz, 1H), 7.96 (s, 1H), 7.83 (d, J=1.9 Hz, 1H), 7.57 (t, J=8.0 Hz, 1H), 7.44-7.41 (m, 2H), 7.37 (d, J=7.8 Hz, 1H), 7.11-7.09 (m, 2H), 6.95 (d, J=8.8 Hz, 1H), 3.63 (m, 4H), 2.88 (m, 4H), 2.14 (s, 3H).

### Synthesis of N-(tert-butyl)-3-((5-methyl-4-((3-(morpholinosulfonyl)phenyl)amino)pyrimidin-2-yl)amino)benzenesulfonamide (M59)

A mixture of intermediate SM-M7 (0.1 g, 0.27 mmol), N-tert-Butyl 3-Aminophenylsulfonamide (CIM55, 0.07 g, 0.32 mmol), Pd₂(dba)₃ (0.02 g, 0.027 mmol), Xantphos (0.047 g, 0.08 mmol) and potassium tert-butoxide (0.06 g, 0.54 mmol) was suspended in dioxane (5 mL) and heated at reflux under a nitrogen atmosphere for 21 hours. The reaction mixture was cooled to room temperature and diluted with CHCl3 (10 mL). The mixture was filtered and the filtrate was concentrated under vacuum. The residue was purified by column chromatography (hexanes:ethyl acetate=5:5) to give the desired product M59 (25 mg, yield: 16%) as a brown solid (see FIG. 16). MS (ESI): m/z 561.16 [M+H]+.

### Synthesis of N-(tert-butyl)-3-((2-((2-methoxy-4-(4-(4-methylpiperazin-1-yl)piperidin-1-yl)phenyl)amino)-5-methylpyrimidin-4-yl)amino)benzenesulfonamide (M60)

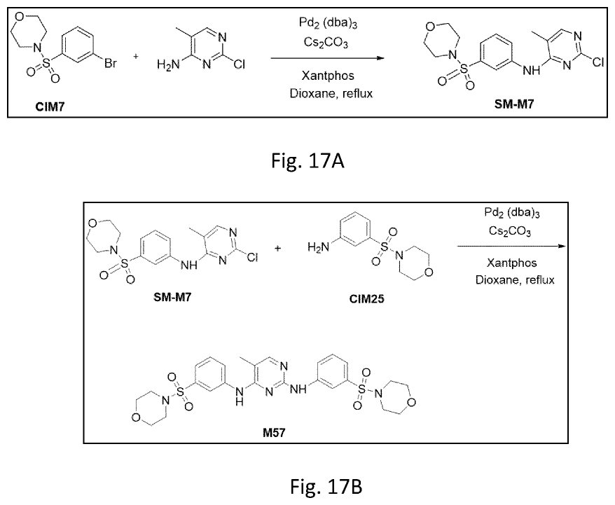

A mixture of intermediate TGo (0.13 g, 0.35 mmol), CIM60 (0.1 g, 0.32 mmol) and trifluoroacetic acid (50 μL) in isopropanol (6 mL) was stirred at 82° C. for 20 h. After completion of the reaction, the solvent was removed and saturated NaHCO₃ solution (15 mL) was added. The aqueous layer was extracted with EtOAc (25 mL×3) and the combined organic layers were dried over MgSO₄, filtered and concentrated under reduced pressure. The residue was purified by column chromatography (CHCl₃:MeOH=9:1) to give the desired product (M60) (20 mg, yield: 10%) as a grey solid (see FIG. 17). MS (ESI): m/z 623.29 [M+H]+.

### Synthesis of N-(tert-butyl)-3-((2-((2-isopropylsulfonyl) phenyl)amino)-5-methylpyrimidin-4-yl))amino)benzenesulfonamide (M62)

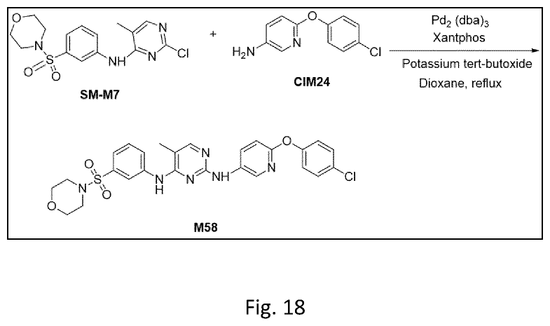

A mixture of TGo (0.1 g, 0.28 mmol), 1-amino-2-(isopropylsulphonyl)benzene (CIM62, 0.07 g, 0.34 mmol), sodium tert-butoxide (0.05 g, 0.56 mmol), Pd₂(dba)₃ (0.025 g, 0.028 mmol), BINAP (0.05 g, 0.08 mmol) in dioxane (5 mL) was prepared under an atmosphere of nitrogen. The resulting mixture was flushed with nitrogen and heated to 105° C. for 24 h. After completion of the reaction, the mixture was then allowed to cool to room temperature and diluted with CHCl3 (10 mL). The mixture was filtered and the filtrate was concentrated under vacuum. The residue was purified by column chromatography (hexane:ethyl acetate=6:4) to give N-(tert-butyl)-3-((2-((2-(isopropylsulfonyl)phenyl)amino)-5-methylpyrimidin-4-yl)amino)benzenesulfonamide (M62) (15 mg, yield: 10%) as a white solid (FIG. 18). MS (ESI): m/z 518. 13 [M+H]+.

### Synthesis of N-(tert-butyl)-3-((5-methyl-2-((3-(4-methyl-1H-imidazol-1-yl)-5-(trifluoromethyl)phenyl)amino)pyrimidin-4-yl)amino)benzenesulfonamide (M64

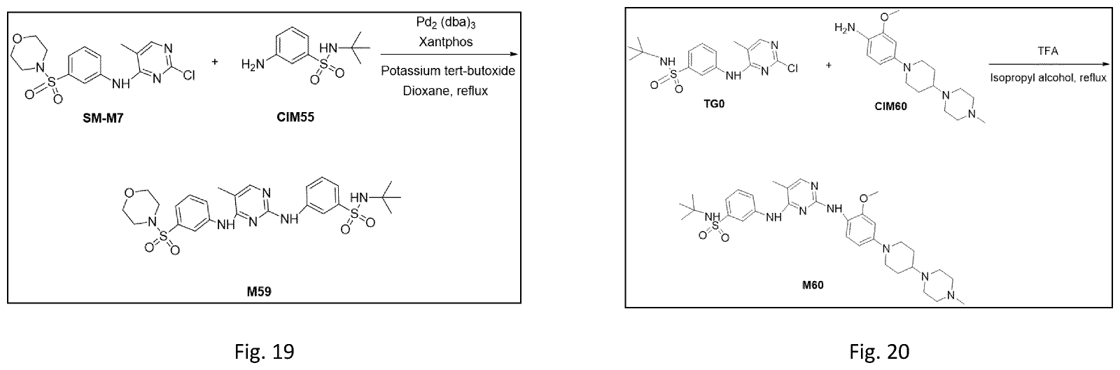

A mixture of intermediate TGo (0.16 g, 0.45 mmol), 3-(4-Methyl-1H-imidazol-1-yl)-5-(trifluoromethyl)aniline (CIM64, 0.1 g, 0.4 mmol) and trifluoroacetic acid (50 μL) in isopropanol/DMF (4:1, 5 mL) was stirred at 100° C. for 24 h. After completion of the reaction, the solvent was removed and saturated NaHCO₃ solution (15 mL) was added. The aqueous layer was extracted with EtOAc (25 mL×3), the combined organic layers were dried over MgSO₄, filtered and concentrated under reduced pressure. The residue was purified by column chromatography (hexane:ethyl acetate=3:7) to give the desired product (M64) (20 mg, yield: 9%) as a yellow-white solid (see FIG. 19). MS (ESI): m/z 560.17 [M+H]+.

### Synthesis of N-(tert-butyl)-3-((2-((2,4-dichloro-5-methoxyphenyl)amino)-5-methylpyrimidin-4-yl)amino)benzenesulfonamide (M69)

A mixture of TGo (0.1 g, 0.28 mmol), 2,4-dichloro-5-methoxyaniline (CIM69, 0.06 g, 0.33 mmol), sodium tert-butoxide (0.05 g, 0.56 mmol), Pd₂(dba)₃ (0.026 g, 0.028 mmol), BINAP (0.05 g, 0.08 mmol) in dioxane (5 mL) was prepared under an atmosphere of nitrogen. The resulting mixture was flushed with nitrogen and heated to 105° C. for 20 h. After completion of the reaction, the mixture was then allowed to cool to room temperature and diluted with CHCl3 (10 mL). The mixture was filtered and the filtrate was concentrated under vacuum. The residue was purified by column chromatography (hexane:ethyl acetate=3:7) to give the desired compound (M69) (25 mg, yield: 18%) as a yellow-white solid (see FIG. 20). MS (ESI): m/z 510. 06 [M+H]+.

### Synthesis of N-(tert-butyl)-3-((2-((3,5-dimethoxyphenyl)amino)-5-methylpyrimidin-4-yl)amino)benzenesulfonamide (M79)

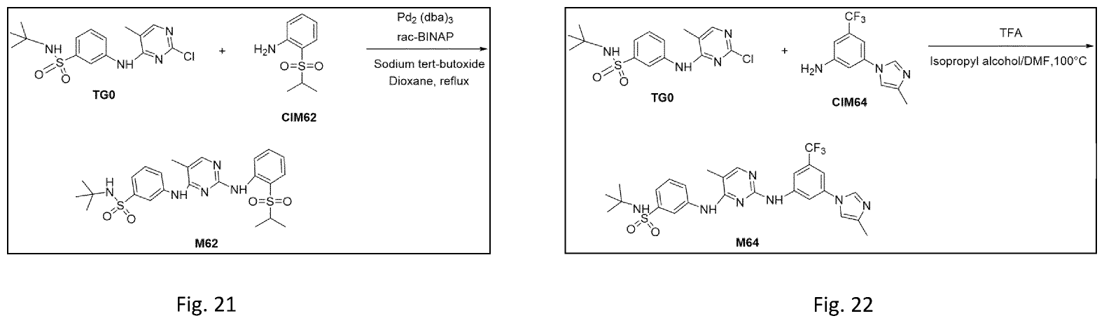

A mixture of intermediate TGo (0.1 g, 0.28 mmol), 3,5-dimethoxyanilineo (CIM79, 0.05 g, 0.34 mmol), Pd₂(dba)₃ (0.018 g, 0.02 mmol), Xantphos (0.024 g, 0.042 mmol) and cesium carbonate (0.18 g, 0.56 mmol) was suspended in dioxane (4 mL) and heated at reflux under a nitrogen atmosphere for 16 hours. The reaction mixture was cooled to room temperature and diluted with DCM (10 mL). The mixture was filtered and the filtrate was concentrated under vacuum. The residue was purified by column chromatography (hexane:ethyl acetate=6:4) to give the desired compound (M79) (30 mg, yield: 22%) as a yellow solid (see FIG. 21). MS (ESI): m/z 472. 20 [M+H]+.

### Synthesis of N-(tert-butyl)-3-((0.5-methyl-2-((4-methyl-3-((4-(pyridin-3-yl)thiazol-2-yl)amino)phenyl)amino)pyrimidin-4-yl)amino)benzenesulfonamide (M88)

A mixture of intermediate TGo (0.15 g, 0.42 mmol), 6-Methyl-N1-(4-(pyridin-3-yl)thiazol-2-yl)benzene-1,3-diamine (CIM88, 0.1 g, 0.35 mmol) and trifluoroacetic acid (55 μL) in Isopropanol/DMF (4/1, 5 mL) was stirred at 100° C. for 24 h. After completion of the reaction, the solvent was removed and saturated NaHCO₃ solution (15 mL) was added. The aqueous layer was extracted with EtOAc (20 mL×3) and the combined organic layers were dried over MgSO₄, filtered and concentrated under reduced pressure. The residue was purified by column chromatography (hexane:ethyl acetate=2:8) to give the desired product M88 (80 mg, yield: 38%) as a light brown solid (see FIG. 22) MS (ESI): m/z 601.20 [M+H]+.

### Synthesis of 4-((4-((3-(N-(tert-butyl)sulfamoyl)phenyl)amino)-5-methylpyrimidin-2-yl)amino)benzoic acid (M101)

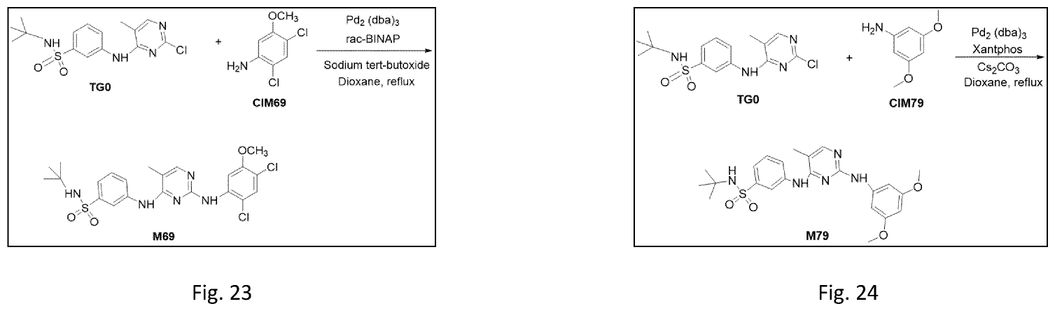

A mixture of intermediate TGo (1.0 g, 2.8 mmol), 4-aminobenzoic acid (CIM101, 0.4 g, 2.9 mmol) and trifluoroacetic acid (0.5 mL) in isopropanol/DMF (8:2, 15 mL) was stirred at 100° C. for 18 h. After completion of the reaction, the solvent was removed and saturated NaHCO₃ solution (20 mL) was added. The aqueous layer was extracted with EtOAc (30 mL×3) and the combined organic layers were dried over MgSO₄, filtered and concentrated under reduced pressure to give the desired product M101 (0.8 g, yield: 63%) as a yellow solid (see FIG. 23). MS (ESI): m/z 456.13 [M+H]+. It was used in the next step without purification.

### Synthesis of tert-butyl 4-(4-((4-((3-(N-(tert-butyl)sulfamoyl)phenyl)amino)-5-methylpyrimidin-2-yl)amino)phenyl)piperazine-1-carboxylate (M102)

A mixture of TGo (1.0 g, 2.8 mmol), tert-butyl 4-(4-aminophenyl)piperazine-1-carboxylate (CIM102, 0.85 g, 3.1 mmol), cesium carbonate (1.8 g, 5.6 mmol), (Pd(OAc)₂)₃ (0.06 g, 0.28 mmol), BINAP (0.35 g, 0.56 mmol) in dioxane (15 mL) was prepared under an atmosphere of nitrogen. The resulting mixture was flushed with nitrogen and heated to 105° C. for 15 h. After completion of the reaction, the mixture was allowed to cool to room temperature and was then diluted with CHCl3 (10 mL). The mixture was filtered and the filtrate was concentrated under vacuum. The residue was purified by column chromatography (hexane:ethyl acetate=5:5) to give M102 (1.0 g, yield: 60%) as a white solid (see FIG. 24). MS (ESI): m/z 596. 23 [M+H]+.

### Synthesis of N-(tert-butyl)-3-((5-methyl-2-((4-(3-morpholinopropoxy)phenyl)amino)pyrimidin-4-yl)amino)benzenesulfonamide (M103)

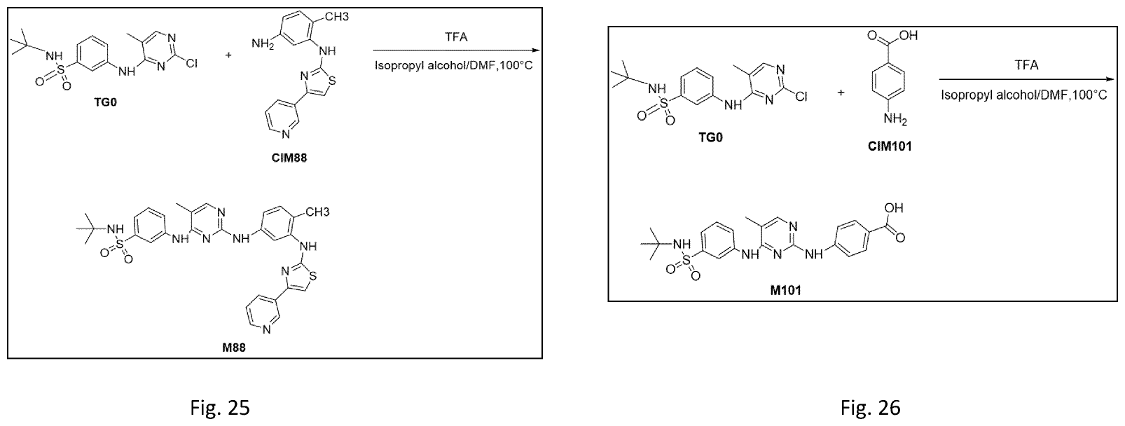

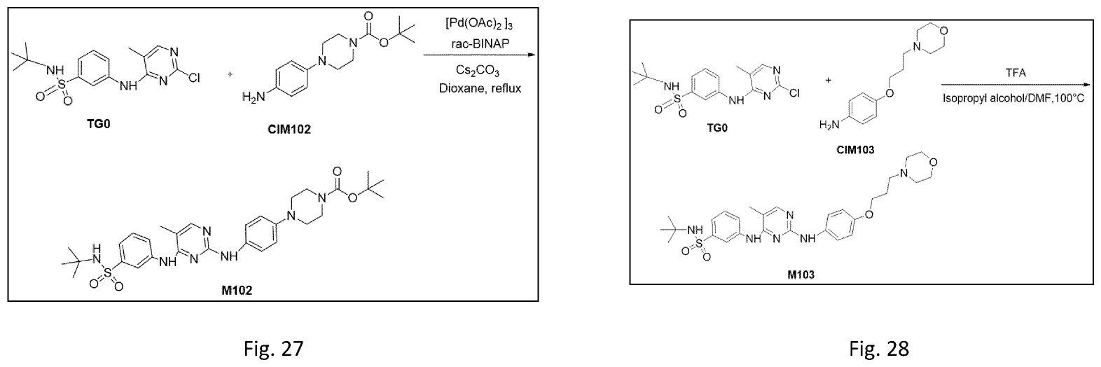

A mixture of intermediate TGo (0.1 g, 0.28 mmol), 4-(3-Morpholinopropoxy)aniline (CIM103, 0.07 g, 0.3 mmol) and trifluoroacetic acid (43 μL) in isopropanol/DMF (4:1, 5 mL) was stirred at 100° C. for 22 h. After completion of the reaction, the solvent was removed and saturated NaHCO₃ solution (15 mL) was added. The aqueous layer was extracted with EtOAc (20 mL×3) and the combined organic layers were dried over MgSO4, filtered and concentrated under reduced pressure. The residue was purified by column chromatography (hexane:ethyl acetate=4 6) to give the desired product M103 (70 mg, yield: 44%) as a white solid (see FIG. 25). MS (ESI): m/z 555.22 [M+H]+.

### Synthesis of N-(tert-butyl)-3-((5-methyl-2-((4-methyl-3-sulfamoylphenyl)amino)pyrimidin-4-yl)amino)benzenesulfonamide (M66)

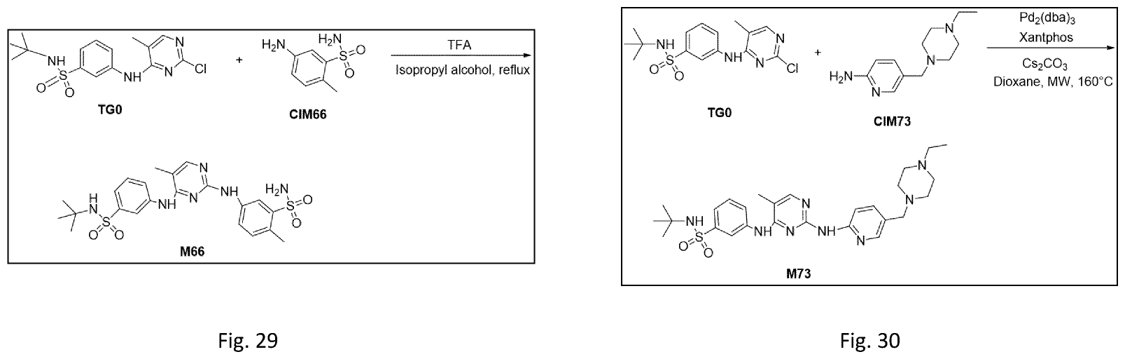

A mixture of intermediate TGO (0.1 g, 0.28 mmol), 5-amino-2-methylbenzenesulfonamide (CIM66, 0.05 g, 0.29 mmol) and trifluoroacetic acid (43 μL) in isopropanol (5 mL) was stirred at 83° C. for 15 h. After completion of the reaction, the solvent was removed and saturated NaHCO₃ solution (15 mL) was added. The aqueous layer was extracted with CHCl₃ (20 mL×3) and the combined organic layers were dried over MgSO4, filtered and concentrated under reduced pressure. The residue was purified by column chromatography (hexane:ethyl acetate=2:8) to give the desired product M66 (40 mg, yield: 28%) as a white solid (see FIG. 29). MS (ESI): m/z 505.03 [M+H]+.

### Synthesis of N-(tert-butyl)-3-((2-((5-((4-ethylpiperazin-1-yl)methyl)pyridin-2-yl)amino)-5-methylpyrimidin-4-yl)amino)benzenesulfonamide (M73)

TGo (0.12 g, 0.34 mmol), 5-((4-ethylpiperazin-1-yl)methyl)pyridin-2-amine (CIM73, 0.08 g, 0.36 mmol), Pd₂(dba)₃ (0.02 g, 0.024 mmol), Xantphos (0.03 g, 0.05 mmol) and cesium carbonate (0.18 g, 0.56 mmol) were suspended in dioxane (5 mL) under a nitrogen atmosphere. The reaction mixture was heated with stirring in a microwave reactor at 160° C. for 15 min. After completion of the reaction, the mixture was diluted with CHCl3 (10 mL), filtered and the filtrate was concentrated under vacuum. The residue was purified by column chromatography (chloroform:methanol=8:2) to give the desired compound M73 (30 mg, yield: 16%) as a yellow solid (see FIG. 30). MS (ESI): m/z 539. 17 [M+H]+.

### Synthesis of 4-((4-((3-(N-(tert-butyl)sulfamoyl)phenyl)amino)-5-methylpyrimidin-2-yl)amino)—N-(3,5-dimethoxyphenyl)benzamide (M10A)

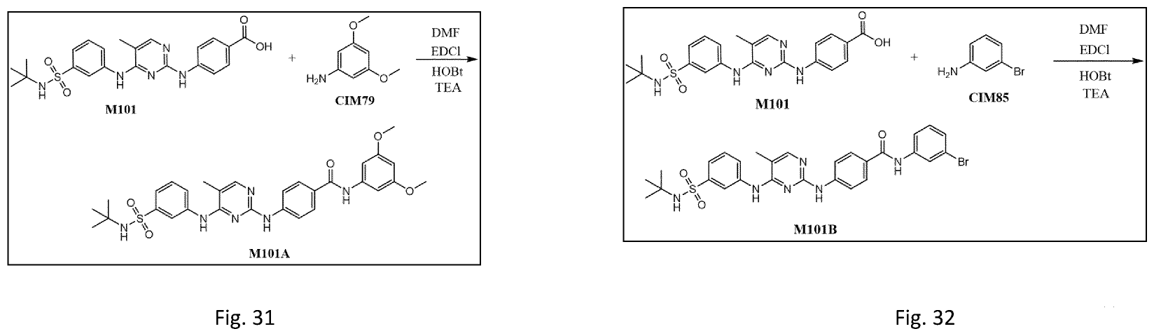

A solution of intermediate M101 (0.15 g, 0.33 mmol), EDCI (0.08 g, 0.6 mmol), HOBt (0.067 g, 0.5 mmol), and triethylamine (TEA) (0.07 mL, 0.52 mmol) in DMF (5 mL) was stirred for 30 min at 0° C., after which 3, 5-dimethoxyaniline (CIM79, 60 mg, 0.4 mmol) was added. The mixture was stirred at room temperature for 20 h. After completion of the reaction, the mixture was poured into ice water and extracted with DCM. The combined organic layers were concentrated and the resulting residue was purified by chromatography on a silica gel column (ethyl acetate:hexane=6:4) to give the desired compound M101A (20 mg, yield: 10%) as a white solid (see FIG. 31). MS (ESI): m/z 591.12 [M+H]+.

### Synthesis of N-(3-bromophenyl)-4-((4-((3-(N-(tert-butyl)sulfamoyl)phenyl)amino)-5-methylpyrimidin-2-Yl)amino)benzamide (M101B)

A solution of intermediate M101 (0.15 g, 0.33 mmol), EDCI (0.08 g, 0.6 mmol), HOBt (0.067 g, 0.5 mmol), and triethylamine (TEA) (0.07 mL, 0.52 mmol) in DMF (5 mL) was stirred for 30 min at 0° C., after which 3-bromoaniline (CIM85, 68 mg, 0.4 mmol) was added. The mixture was stirred at room temperature for 20 h. After completion of the reaction, the mixture was poured into ice water and extracted with DCM. The combined organic layers were concentrated, and the resulting residue was purified by chromatography on a silica gel column (ethyl acetate:hexane=6:4) to give the desired compound M101B (55 mg, yield: 27.5%) as a white solid (see FIG. 32). MS (ESI): m/z 610.97 [M+H]+.

### Synthesis of N-(3-(N-(tert-butyl)sulfamoyl)phenyl)-4-((4-((3-(N-(tert-butyl)sulfamoyl)phenyl)amino)-5-methylpyrimidin-2-yl)amino)benzamide (M101D

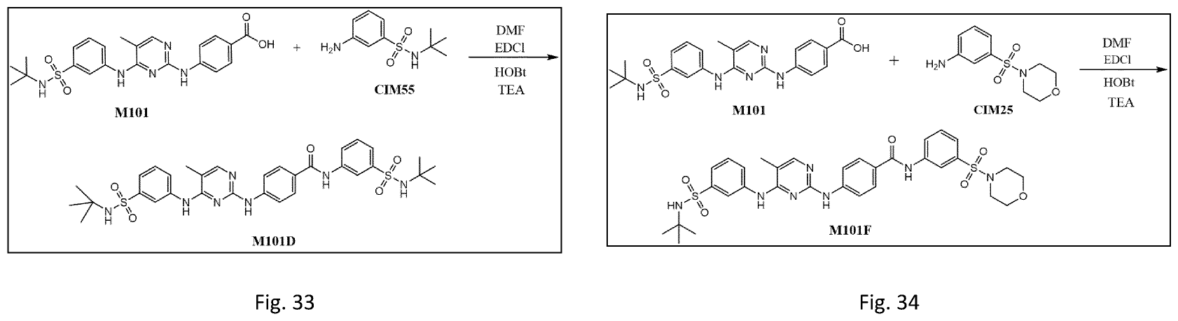

A solution of intermediate M101 (0.15 g, 0.33 mmol), EDCI (0.08 g, 0.6 mmol), HOBt (0.067 g, 0.5 mmol), and triethylamine (TEA) (0.07 mL, 0.52 mmol) in DMF (5 mL) was stirred for 30 min at 0° C., after which 3-amino-N-(tert-butyl)benzenesulfonamide CIM55 (92 mg, 0.4 mmol) was added. The mixture was stirred at room temperature for 16 h, after completion of the reaction, the mixture was poured into ice water and extracted with DCM. The combined organic layers were concentrated and the resulting residue was purified by chromatography on a silica gel column (Ethyl acetate:hexane=7:3) to give the desired compound M101D (15 mg, yield: 7%) as a white solid (see FIG. 33). MS (ESI): m/z 666.19 [M+H]+.

### Synthesis of 4-((4-((3-(N-(tert-butyl)sulfamoyl)phenyl)amino)-5-methylpyrimidin-2-yl)amino)—N-(3-(morpholinosulfonyl)phenyl)benzamide

A solution of intermediate M101 (0.15 g, 0.33 mmol), EDCI (0.08 g, 0.5 mmol), HOBt (0.067 g, 0.6 mmol), and triethylamine (TEA) (0.07 mL, 0.5 mmol) in DMF (5 mL) was stirred for 30 min at 0° C., after which 3-(morpholinosulfonyl)aniline (CIM25, 95 mg, 0.4 mmol) was added. The mixture was stirred at room temperature for 24 h, after completion of the reaction, the mixture was poured into ice water and extracted with DCM. The combined organic layers were concentrated and the resulting residue was purified by chromatography on a silica gel column (Ethyl acetate:hexane=8:2) to give the desired compound M101F (18 mg, yield: 8%) as a white solid (see FIG. 34). MS (ESI): m/z 680.49 [M+H]+.

### Synthesis of 4-((4-((3-(N-(tert-butyl)sulfamoyl)phenyl)amino)-5-methylpyrimidin-2-yl)amino)—N-(4-(3-morpholinopropoxy)phenyl)benzamide M101G

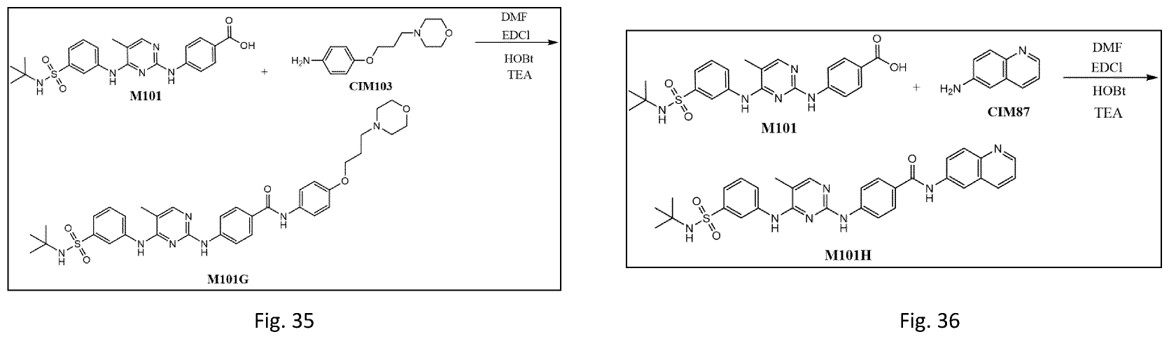

A solution of intermediate M101 (0.15 g, 0.33 mmol), EDCI (0.08 g, 0.5 mmol), HOBt (0.067 g, 0.5 mmol), and triethylamine (TEA) (0.07 mL, 0.5 mmol) in DMF (5 mL) was stirred for 30 min at 0° C., after which 4-(3-morpholinopropoxy)aniline (CIM103, 93 mg, 0.4 mmol) was added. The mixture was stirred at room temperature for 20 h. After completion of the reaction, the mixture was poured into ice water, and extracted with DCM. The combined organic layers were concentrated and the resulting residue was purified by chromatography on a silica gel column (chloroform:methanol=9:1) to give the desired compound M101G (80 mg, yield: 36%) as a white solid (see FIG. 35). MS (ESI): m/z 674.45 [M+H]+.

### Synthesis of 4-((4-((3-(N-(tert-butyl)sulfamoyl)phenyl)amino)-5-methylpyrimidin-2-yl)amino)—N-(quinolin-6-yl)benzamide (M101H)

A solution of intermediate M101 (0.15 g, 0.33 mmol), EDCI (0.08 g, 0.5 mmol), HOBt (0.067 g, 0.5 mmol), and triethylamine (TEA) (0.07 mL, 0.5 mmol) in DMF (5 mL) was stirred for 30 min at 0° C., after which quinolin-6-amine (CIM87, 57 mg, 0.4 mmol) was added. The mixture was stirred at room temperature for 24 h. After completion of the reaction, the mixture was poured into ice water and extracted with DCM. The combined organic layers were concentrated, and the resulting residue was purified by chromatography on a silica gel column (chloroform:methanol=9:1) to give the desired compound M101H (30 mg, yield: 15%) as a white solid (see FIG. 36). MS (ESI): m/z 582.11 [M+H]+.

### Synthesis of N2-(3,5-Dimethoxyphenyl)-5-methyl-N4-(3-(morpholinosulfonyl)phenyl)pyrimidine-2,4-diamine (M104)

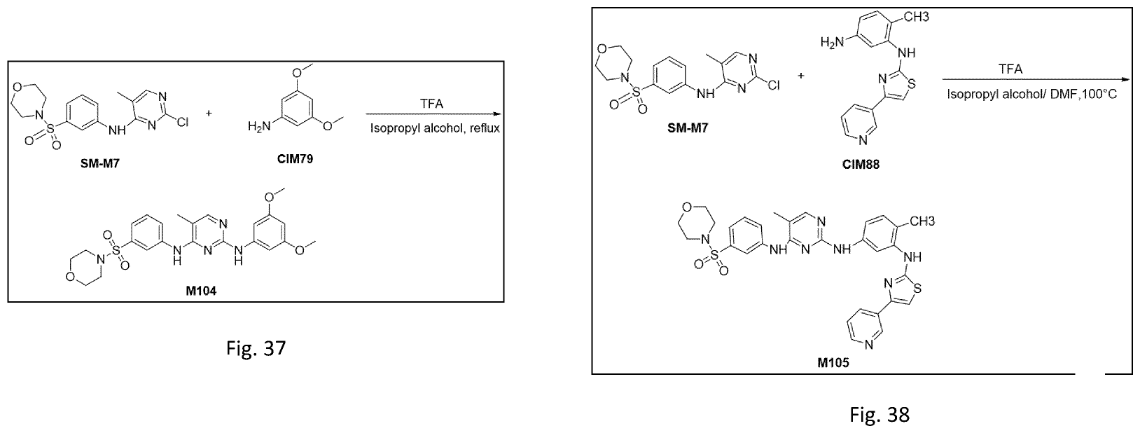

A mixture of intermediate SM-M7 (0.1 g, 0.27 mmol), 3,5-dimethoxyaniline (CIM79, 0.045 g, 0.29 mmol) and trifluoroacetic acid (42 L) in isopropanol (5 mL) was stirred at 83° C. for 16 h. After completion of the reaction, the solvent was removed and saturated NaHCO₃ solution (15 mL) was added. The aqueous layer was extracted with CHCl₃ (20 mL×3) and the combined organic layers were dried over MgSO4, filtered and concentrated under reduced pressure. The residue was purified by column chromatography (hexane:ethyl acetate=4:6) to give the desired product M104 (35 mg, yield: 27%) as a yellow solid (see FIG. 37). MS (ESI): m/z 486.04 [M+H]+.

### Synthesis of 5-methyl-N2-(4-methyl-3-((4-(pyridin-3-yl)thiazol-2-yl)amino)phenyl)-N4-(3-(morpholinosulfonyl)phenyl)pyrimidine-2,4-diamine (M105)

A mixture of intermediate SM-M7 (0.1 g, 0.27 mmol), 6-methyl-N1-(4-(pyridin-3-yl)thiazol-2-yl)benzene-1,3-diamine (CIM88, 0.084 g, 0.3 mmol), and trifluoroacetic acid (42 L) in isopropanol/DMF (4:1, 5 mL) was stirred at 100° C. for 14 h. After completion of the reaction, the solvent was removed and saturated NaHCO₃ solution (15 mL) was added. The aqueous layer was extracted with CHCl₃ (20 mL×3) and the combined organic layers were dried over MgSO4, filtered, concentrated under reduced pressure and the reaction mixture was subjected to flash chromatography on silica gel (hexane:ethyl acetate=1:9) to afford (30 mg, yield: 19%) of 105 as a light brown solid (see FIG. 38). MS (ESI): m/z 615.12 [M+H]+.

### Synthesis of 5-methyl-N2-(4-(3-morpholinopropoxy)phenyl)-N4-(3-(morpholinosulfonyl)phenyl)pyrimidine-2,4-diamine (M106)

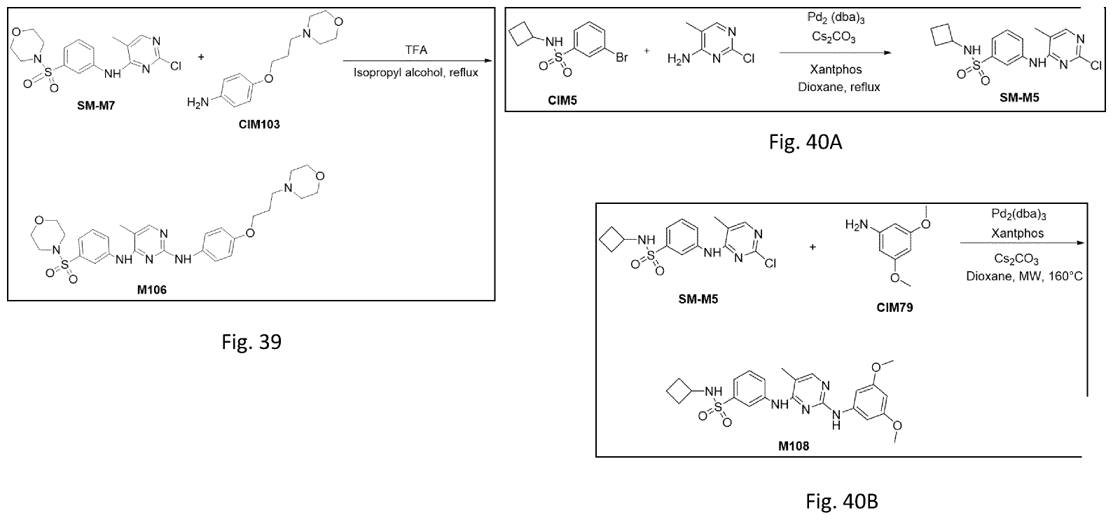

A mixture of intermediate SM-M7 (0.1 g, 0.27 mmol), 4-(3-morpholinopropoxy)aniline (CIM103, 0.065 g, 0.28 mmol) and trifluoroacetic acid (42 μL) in isopropanol (5 mL) was stirred at 83° C. for 16 h. After completion of the reaction, the solvent was removed and saturated NaHCO₃ solution (15 mL) was added. The aqueous layer was extracted with CHCl₃ (20 mL×3) and the combined organic layers were dried over MgSO4, filtered and concentrated under reduced pressure. The residue was purified by column chromatography (chloroform:methanol=9:1) to obtain (20 mg, yield: 13%) of M106 as a white solid (see FIG. 39). MS (ESI): m/z 569.17 [M+H]+.

### Synthesis of N-cyclobutyl-3-((2-((3,5-dimethoxyphenyl)amino)-5-methylpyrimidin-4-yl)amino)benzenesulfonamide (108)

Step 1: Cesium carbonate (1.8 g, 5.6 mmol), tri(dibenzylacetone)diphenyl palladium (0.18 g, 0.2 mmol) and 4,5-diphenylphosphine-9,9-dimethoxyanthracene (0.24 g, 0.4 mmol) was added to a solution of 3-bromo-N-cyclobutylbenzenesulfonamidee (CIM5, 0.9 g, 3.1 mmol) and 2-chloro-5-methylpyrimidin-4-amine (0.4 g, 2.8 mmol) in 1,4-dioxane (10 mL). The mixture was stirred at 105° C. for 7 hours under N₂ atmosphere. The reaction mixture was cooled to room temperature and diluted with CHCl3 (15 mL). The mixture was filtered and the filtrate concentrated in vacuo. The residue was dissolved in EtOAc and hexane was added until solid precipitation occurred. After filtration, SM-M5 (0.75 g, 70%) was obtained as an orange solid (see FIG. 40A). MS (ESI): m/z 351.14 [M−H]−. It was used in the next step without purification.

Step 2: SM-M5 (0.1 g, 0.28 mmol), 3,5-dimethoxyaniline (CIM79, 0.05 g, 0.3 mmol), Pd₂(dba)₃ (0.018 g, 0.02 mmol), Xantphos (0.024 g, 0.042 mmol) and cesium carbonate (0.18 g, 0.56 mmol) were suspended in dioxane (5 mL) under a nitrogen atmosphere. The reaction mixture was heated with stirring in a microwave reactor at 160° C. for 15 min. After completion of the reaction, the mixture was diluted with CHCl3 (10 mL), filtered and the filtrate was concentrated under vacuum. The residue was purified by column chromatography (hexane:ethyl acetate=5:5) to give the desired compound M108 (10 mg, yield: 7%) as a yellow solid (see FIG. 40B). MS (ESI): m/z 470. 10 [M+H]+.

### Examples—Results

Identification of ccRCC Signature Genes

Both Kaplan-Meier analysis and univariate Cox model were applied to investigate the associations of the mRNA expression levels of genes with the patients' OS in TCGA and Japanese ccRCC cohorts. A Kaplan-Meier survival analysis was performed by classifying the patients into two groups with high and low expression (TPM values) of the investigated gene by selecting an optimal cut off from the 20^{th} and 80^{th} expression percentiles yielding the lowest log-rank P-value as in our previous study[18,19]. Meanwhile, the univariate Cox analysis was performed by calculating the hazard ratio of each gene. As a result, an overlap of 7,813 prognostic genes was identified by Kaplan-Meier and Cox survival analysis in the TCGA cohort (FDR<0.05). Similarly, 1,335 prognostic genes were identified in the Japanese cohort (FDR<0.05). The two sets of genes had a significant overlap (n=1,129, hypergeometric distribution test, P<1.Ile-16), and the concordance score of these overlapped genes (both favorable or unfavorable genes) is 99.91%. Among these 1,129 prognostic genes, there were 342 unfavorable genes and 787 favorable genes whose high expression indicated poor and good survival outcomes of patients, respectively. Functional enrichment analysis showed that the unfavorable genes were significantly enriched in cell division and cell cycle pathways. In contrast, the favorable genes were significantly enriched in the angiogenesis, vasculogenesis, cell migration and cell differentiation pathways (FDR<0.05), which are well-known hallmarks in cancer [17]. Therefore, these genes were used as unfavorable and favorable signature genes for ccRCC.

### Identification of Functional Modules in the GCN

GCN analysis is a powerful method to identify the hub genes that drive the key cellular signaling pathways, including a set of co-expressed or functionally associated genes during tumor development. The conservative co-expressed pattern may confer a selective advantage for tumor cells if it could be validated in independent datasets [20,21]. The Spearman correlation between two possible gene pairs based on the mRNA expression profiles of ccRCC patients in TCGA and Japanese cohort, respectively, was calculated. To balance the sensitivity for detecting the functional gene modules and the robustness for validation in independent datasets, gene-gene links involved in the top 1% with the highest correlation coefficients were extracted to build the GCN. Around 900,000 gene-gene links with correlation coefficients ranging from 0.74 to 1 were obtained in the co-expression network of the TCGA cohort. Then, a random walks-based algorithm, Walktrap[22], was used to identify the gene modules with high transitivity based on the topology of the GCN. To discover functionally meaningful modules in which genes show a good connectivity, the modules with more than 20 genes and clustering coefficients higher than 0.6 were extracted. Finally, 18 modules (M1-18) were obtained in the TCGA cohort. Further, the association of each module with the unfavorable and favorable signature genes was investigated by concordance analysis. It was found that the 41 genes involved in M11 had a significant overlap with the 342 unfavorable signature genes (n=40, hypergeometric test, FDR<0.05). Thus, the M11 was determined as an unfavorable module. Meanwhile, it was observed that the genes involved in M1 (184 genes), M2 (177 genes), M3 (88 genes), M4 (76 genes) and M18 (22 genes) had significant overlaps with the 787 favorable signature genes, respectively (n=51, 86, 33, 18 and 6, hypergeometric test, FDR<0.05), which were determined as favorable modules. Then, the biological function of these modules was investigated by functional enrichment analysis. It was found that the genes involved in unfavorable module were significantly enriched in cell division, cell cycle, DNA replication and MAPK activity pathways (FDR<0.05). The genes involved in favorable modules were significantly enriched in the angiogenesis, vasculogenesis, cell migration, cell differentiation, cell adhesion and several signaling pathways (cAMP-mediated signaling, ERK1 and ERK2 cascade, Ras protein signal transduction and Rho protein signal transduction) (FDR<0.05).

Based on the same methodology, around 900 000 gene-gene links with correlation coefficients ranging from 0.68 to 1 in the GCN of the Japanese cohort were obtained. Five modules (M1-5) were found based on the exact cutoff setting. M3 (81 genes) and M1 (770 genes) had significant overlaps with the unfavorable and favorable signature genes, respectively (n=76 and 222, hypergeometric test, FDR<0.05), which were correspondingly determined as unfavorable and favorable modules. Functional enrichment analysis of the two modules exhibited an identical set of biological pathways resulting from the TCGA cohort. Based on these results, it was inferred that M11 from the TCGA cohort and M3 from the Japanese cohort indicated the same unfavorable gene module and M1, M2, M3, M4 and M18 from the TCGA cohort and M1 from the Japanese cohort indicated the same favorable gene module. The individual genes in these two modules were visualized. The M3 from the Japanese cohort wholly covered all the genes involved M11 from the TCGA cohort, which is highly significant overlap (n=41, hypergeometric test, P<1.1e-16). Moreover, each of M1, M2, M3, M4 and M18 from the TCGA cohort had a significant overlap with M1 from the Japanese cohort (n=98, 168, 78, 73 and 20, hypergeometric, all P<1.1e-16). Thus, the genes from M1, M2, M3, M4 and M18 were merged in the TCGA cohort and generated an integrated module M1/2/3/4/18. These findings suggested that two types of gene modules that lead to unfavorable and favorable survival outcomes of patients in ccRCC were found, which had high confidence since these modules were validated in an independent cohort with different racial and geographical characteristics.

It was found the genes involved in M7 and M17 from the TCGA cohort were significantly enriched in the innate immune response pathways (phagocytosis, macrophage activation and Toll-like receptor signaling pathway) and the genes involved in M5, M6, M12 and M15 were significantly enriched in the adaptive immune response pathways (T/B cell activation, T cell differentiation and B cell receptor signaling pathway). Interestingly, the genes involved in M2 from the Japanese cohort were significantly enriched in an identical set of both innate and adaptive immune response pathways. Concordance analysis showed that each of the modules from the TCGA cohort mentioned above had a significant overlap with M2 from the Japanese cohort, suggesting these immune-related modules identified from the TCGA cohort were also independently validated in the Japanese cohort. In addition, the genes involved in M4 from the Japanese cohort were enriched in the fatty acid metabolic process. A previous study reports three molecular subtypes of ccRCC characterized by the different activity of energy metabolism in terms of cell viability and proliferation, cell differentiation and fatty acid metabolism[14], which are consistent with the functions of the modules identified in this study. The genes involved in M5 from the Japanese cohort were significantly enriched in the translational initiation, protein targeting to the endoplasmic reticulum, protein targeting to membrane, viral transcription and viral gene expression pathways. Interestingly, two of the three subtypes identified in the previous study were characterized by the opposite dysregulation of these pathways, corresponding to patients' best and poorest survival outcomes[14].

### Identification of the Potentially Druggable Target Genes

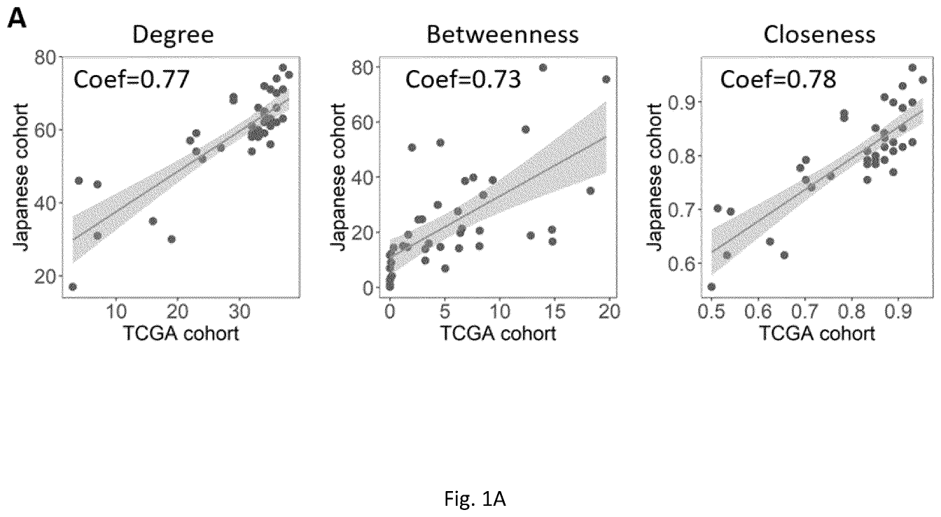

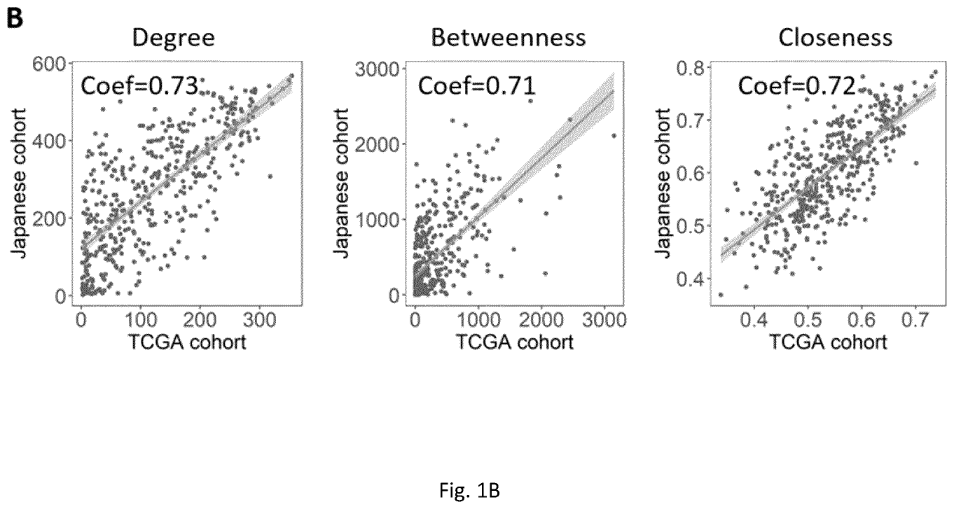

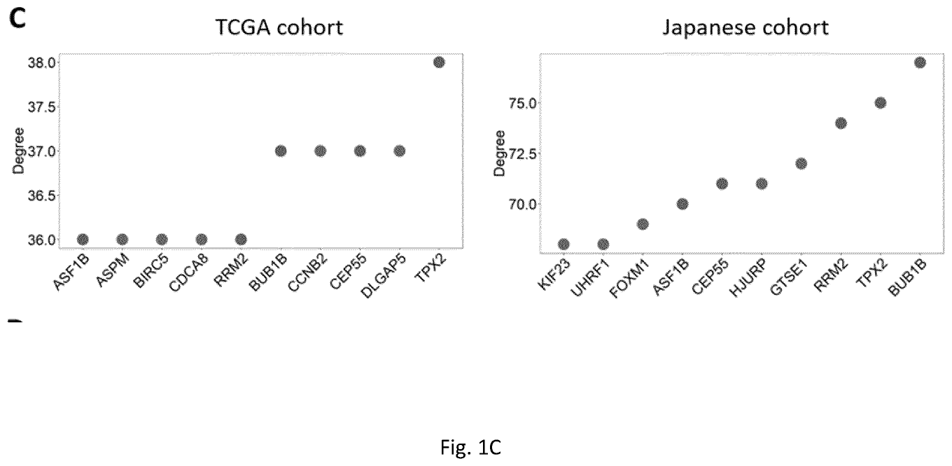

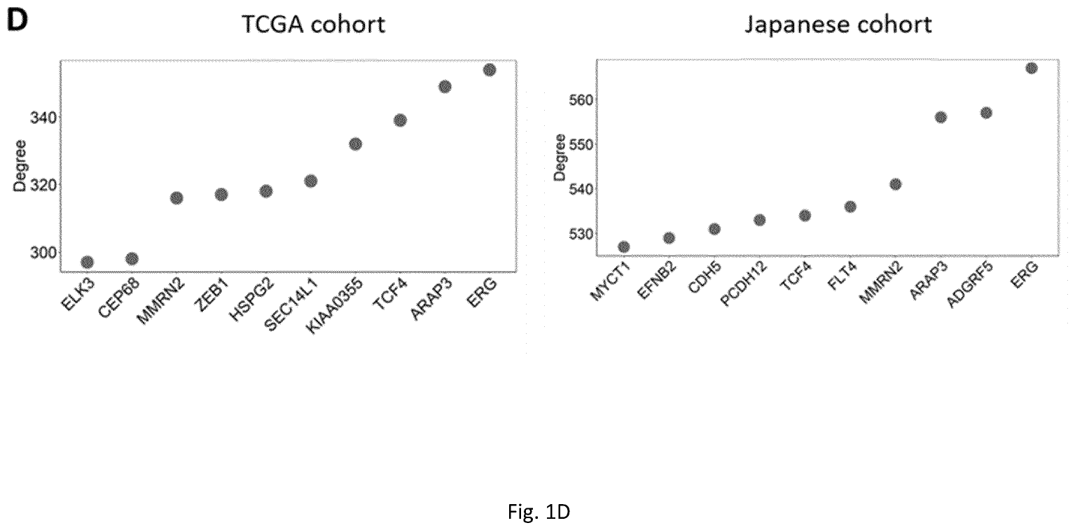

Analysis of network topology could provide insight into the importance of a given gene in the network. The genes with high centrality are considered as hub genes in the network, and it was predicted that these are potential targets that are worthwhile to be further evaluated. The degree, betweenness and closeness of each gene in each unfavourable and favourable module were calculated. These three measurements are commonly used to characterize the centrality of nodes from three distinct perspectives: a higher degree indicates that the node is involved in more interactions; a higher betweenness indicates that the node acts as a bridge which lies on the shortest path between other nodes; a higher closeness indicates that the node shows shorter paths to all the other nodes and is likely to be the geometric center of the module [23-26]. The Spearman correlation of each centrality measurement between the two unfavorable modules (or favorable modules) identified from TCGA and Japanese datasets were then calculated. As shown in FIGS. 1A and 1B, the correlation coefficients of degree, betweenness and closeness were 0.77, 0.73 and 0.78 between M11 from the TCGA cohort and M3 from the Japanese cohort, and 0.73, 0.71 and 0.72 between M1/2/3/4/18 from TCGA cohort and M1 from the Japanese cohort. This result suggests that the topology of the unfavorable and favorable modules identified from the TCGA cohort are highly similar to the Japanese cohort even though the two cohorts have different racial and geographical characteristics. The genes involved in each unfavourable and favourable module were ranked based on decreasing order of the degree, betweenness and closeness values. Then the common genes involved in the top 10 gene lists with the highest rank were selected based on at least one of the three centrality measurements in both TCGA and Japanese cohorts as the hub genes. Consequently, six hub genes, namely BUB1B, TPX2, RRM2, CEP55, ASF1B and CCNB2, from the two unfavorable modules (FIG. 1C) and four hub genes, including ERG, ARAP3, TCF4 and MMRN2, from the two favorable modules (FIG. 1D) were found. Here CD93 was ignored in the favorable modules since it had no significant association with prognoses of patients. The prognostic effects of these hub genes have been established (data not shown).

The expression levels of the 10 hub genes were visualized based on the gene expression profiles of TCGA tumors and adjacent normal samples. As shown in FIGS. 2A and 2B, most of these hub genes, especially the six unfavorable hub genes, presented very low expression in normal tissues, while their expression levels were significantly increased in the tumor tissues. As these unfavorable hub genes encode cell mitosis and cell cycle-related proteins, this alteration may indicate that low expression of these genes maintains normal cell renewal and metabolism, but increased expression promotes tumorigenesis or tumor progression. Meanwhile, the significantly increased expression levels of favorable hub genes in tumor tissue may imply activating a protective mechanism to mediate anti-tumor effect in cancer. Further, the altered coverage of these hub genes was investigated in TCGA tumor tissues. The result showed 84-96% of patients presented a high expression of unfavorable hub genes, which leads to poor survival outcomes of patients and 59-73% of patients showed an increased expression of favorable genes, which leads to good survival of patients (FIG. 2C), suggesting that most of the ccRCC patients may carry the common molecular alterations induced by the unfavorable hub genes but not for their anti-tumor mechanisms. To confirm the expression changes at the protein level, the expression differences observed at the mRNA level were validated using the immunohistochemistry (IHC) images of the hub genes in normal tissue and tumor tissue from the Human Protein Atlas [27,28](FIG. 2D). Four hub genes with selected antibodies showed a higher protein expression in tumor cells than corresponding normal tissues. For example, TPX2 was not detected in normal glomeruli cells, and lowly expressed in normal tubule cell, but showed medium expression in tumor cells (FIG. 2D).

Moreover, the essential scores of these hub genes in 16 ccRCC cell lines were extracted from the Dependency Map (DepMap) portal[29]. The essential scores were evaluated based on genome-scale CRISPR-Cas9 loss-of-function screens and corrected by a computational method CERES[29]. A more negative score of a gene indicates this gene is more essential for tumor cell proliferation and survival. As shown in FIG. 2E, most of the unfavorable hub genes were more essential than the favorable genes in ccRCC. The mRNA expression levels of these hub genes were also investigated in Caki-1, a commonly used ccRCC cell model. The result showed that the unfavorable hub genes had appropriate expression levels in Caki-1 (FIG. 2F). Considering these, the unfavorable hub genes are considered more appropriate target genes, and the inhibitory effect on these genes (e.g., shRNAs and chemical inhibitors) should be further evaluated.

### Drug Repositioning for Target Genes

To find the potential therapeutic drugs for candidate targets, a drug repositioning approach was developed based on an integrated analysis of the drug- and shRNA-perturbed signature profiles from the CMap database[30]. It was hypothesized that a drug has an inhibited effect on the expression of a target gene in tumor cells if the drug treatment leads to a similar dysregulation of gene expression induced by shRNA knockdown. In brief, the drug repositioning approach consists of the following four steps (see Method section for details): 1) Constructing the drug-shRNA matrix for each target gene. The drug-perturbed and shRNA-perturbed signature matrix of the HA1E cell line, the only kidney cell line in CMap database, was extracted. TPX2 was not analyzed in the following analysis because its shRNA knockdown data was not provided in CMap. For each of the other five target genes, a correlation matrix, namely drug-shRNA matrix, was constructed by calculating Spearman correlation between all possible combinations of drug-perturbed and target-specific shRNA-perturbed signatures. The correlation coefficients in this matrix represent the similarity of effects on gene expression induced by specific drug treatment and specific shRNA knockdown. 2) Optimizing the drug-shRNA matrix. For a specific drug, different doses and treated time points were set for cell line treatment in the CMap experiments. Thus, several perturbed profiles associated with the same drug treatment in a cell line were usually obtained. It has been reported that the effects of drug on human cells is highly dependent on the dose setting [31]. Under different doses, drug-target binding affinities could be changed and the downstream pathways can be affected differently, especially for cell cycle related pathways [31]. To maximally represent the drug efficacy, only the optimal dose and time point with the highest similarity with each shRNA knockdown was kept. For a specific gene knockdown experiment, different shRNAs were set for targeting the same gene. These shRNAs are the specific sub-sequences from the investigated gene, whose sequences could be completely different or share some common bases. To maximally represent the gene knockdown efficacy and avoid the effects from the off-target shRNAs, at least two shRNAs which showed a consistently higher correlation with drug treatment were extracted by a clustering analysis. As a result, five simplified drug-shRNA matrixes were obtained and each of the matrixes had 6,986 drugs (rows) while three shRNAs (columns) for BUB1B, two shRNA for RRM2, two shRNA for CEP55, two shRNA for ASF1B, and four shRNA for CCNB2, respectively. 3) Extracting the top 1% drug candidates. In a given drug-shRNA matrix, the drugs were ranked based on the correlation with each shRNA. The overlapped drugs, which ranked in the top 1% (70) drug lists with the highest correlation with each shRNA as candidates were extracted. 4) Selecting the three most effective drugs for each target. The top three drugs with the highest median rank across different shRNAs were finally considered as the most effective drugs for each target. As shown in FIG. 3, it was discovered that TG-101209, oxetane, WH-4-025 target BUB1B, NVP-TAE684, MK-0752, and withaferin-a target RRM2, actinomycin-d, triphenyl-tin, RS—I-002-6 target CEP55, BRD-K26510616, panobinostat and tacedinaline target ASF1B and oxetane and BRD-K54343811 target CCNB2.

### Validation of Target Genes and Drug Effect

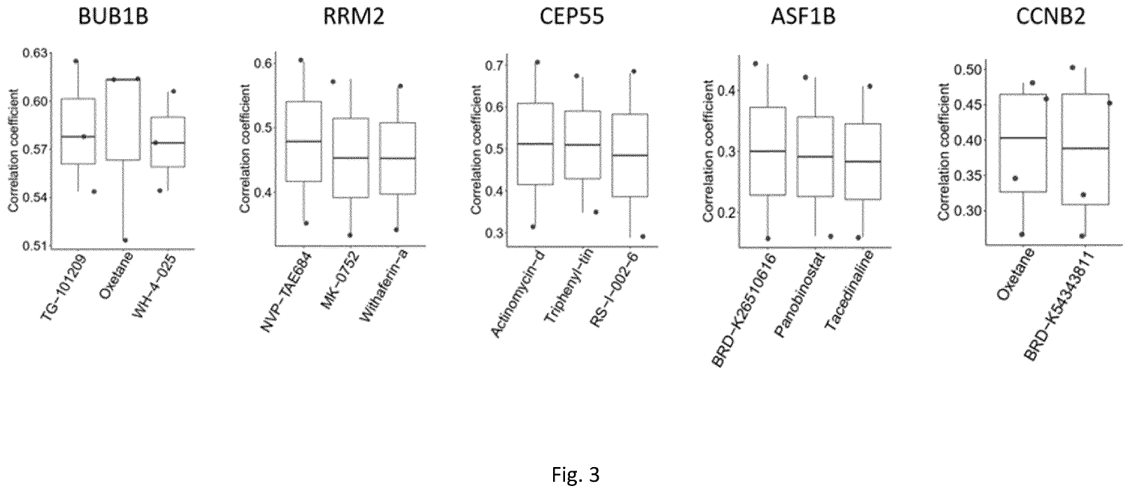

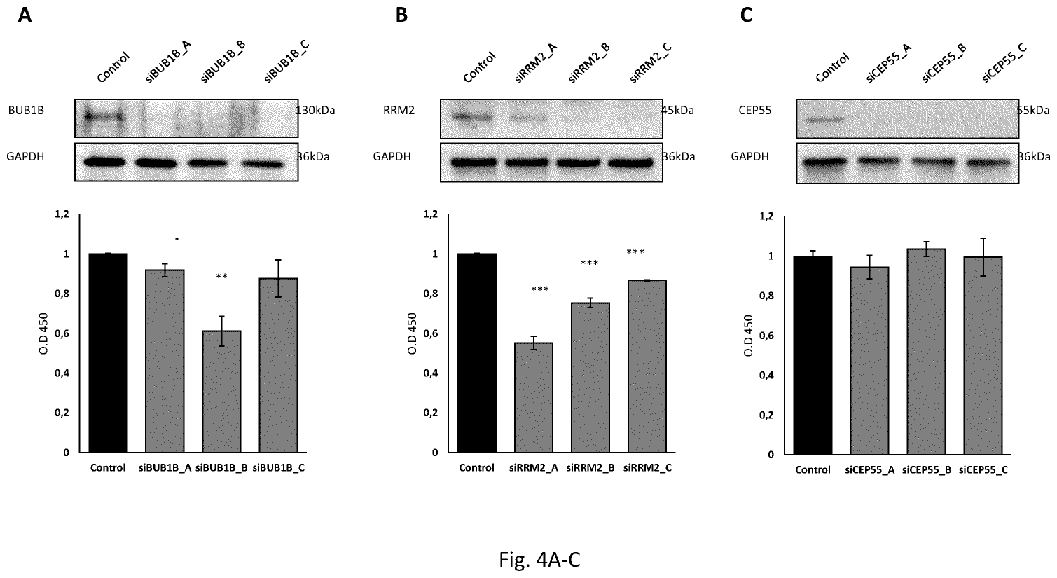

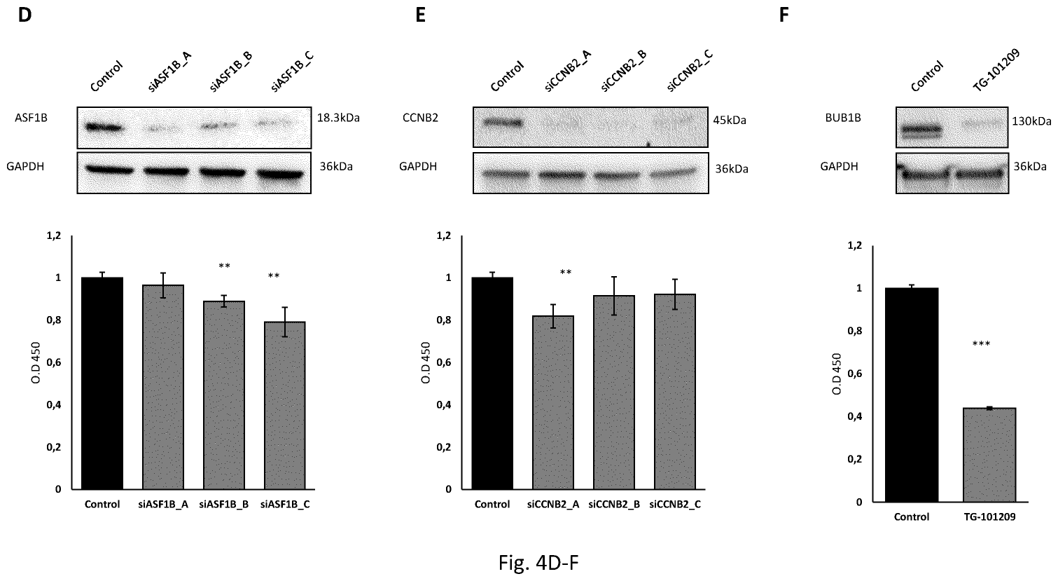

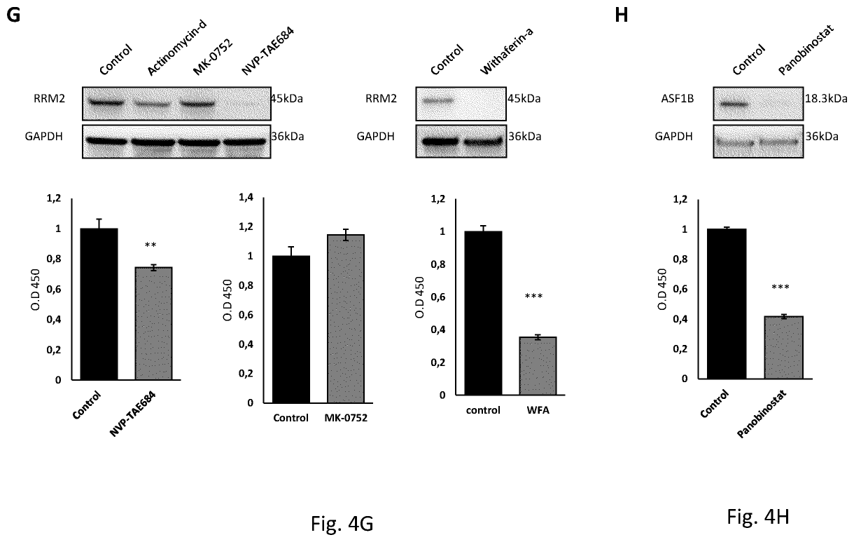

To confirm the essentiality of the target genes in ccRCC, the expression of target genes was inhibited by transfecting siRNAs to Caki-1 and investigated the effect on cell viability. Western blots showed that the protein expression of the five target genes was successfully suppressed in Caki-1 transfected with siRNAs compared with negative control (FIG. 4A-E). A CCK-8 assay was performed to measure cell proliferation. The result indicated that the cell viability was significantly decreased by the knockdown of BUB1B, RRM2, ASF1B and CCNB2 in Caki-1 but not for the knockdown of CEP55 (FIG. 4A-E). Thus, CEP55 was excluded in the following experiments.

Further, it was investigated whether the repurposed drugs could inhibit their corresponding target genes. TG-101209, NVP-TAE684, MK-0752, withaferin-a, actinomycin-d, and panobinostat were purchased, and these drugs were tested in Caki-1 in the following experiments. As shown in FIG. 4F, it was observed that the protein level of BUB1B was significantly decreased by the treatment of TG-101209, which was identified as the most effective drug for BUB1B compared to the negative control, and the cell viability was also significantly reduced. NVP-TAE684, MK-0752, and withaferin-a were the top three effective drugs that were identified for targeting RRM2. As shown in FIG. 4G, it was observed that the protein level of RRM2 was significantly suppressed by both NVA-TAE684 and withaferin-a, but not by MK-0752. The cell viability was significantly reduced by NVA-TAE684 and withaferin-a but not by MK-0752, consistent with the result showed in Western blots. The effect of actinomycin-d (actually targeting CEP55 based on our prediction) on RRM2 was also tested. The Western blot showed that this drug does not work for inhibiting RRM2. The protein level of ASF1B was significantly decreased by its target drug panobinostat and cell viability was also significantly reduced after the drug treatment (FIG. 4H). In summary, BUB1B, RRM2 and ASF1B were confirmed as druggable targets and drugs (i.e. TG-101209, NVP-TAE684, withaferin-a and panobinostat) targeting them have been identified and validated in vitro.

### Development of TG-101209 Derivatives

As treatment with TG-101209 significantly reduced the expression levels of BUB1B and the cell viability of treated cells, alternative molecules based on the molecular structure of TG-101209 were developed, see Table 1.

Table 1 shows the synthesized TG-101209 derivatives.

| Sample name | Molecular structure |
|---|---|
| M1 | 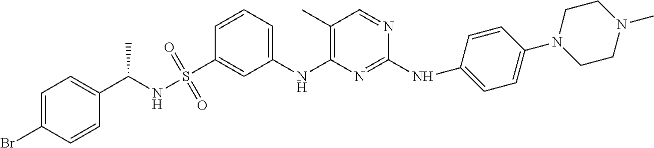 [SMILES: Cc1cnc(Nc2ccc(N3CCN(C)CC3)cc2)nc1Nc1cccc(S(=O)(=O)N[C@@H](C)c2ccc(Br)cc2)c1] |
| M3 | 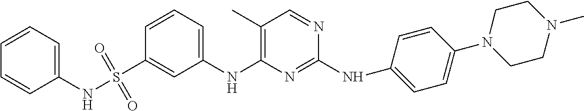 [SMILES: Cc1cnc(Nc2ccc(N3CCN(C)CC3)cc2)nc1Nc1cccc(S(=O)(=O)Nc2ccccc2)c1] |
| M5 | 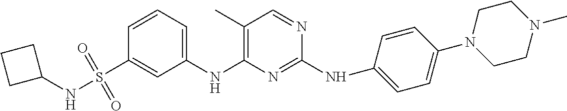 [SMILES: Cc1cnc(Nc2ccc(N3CCN(C)CC3)cc2)nc1Nc1cccc(S(=O)(=O)NC2CCC2)c1] |
| M7 | 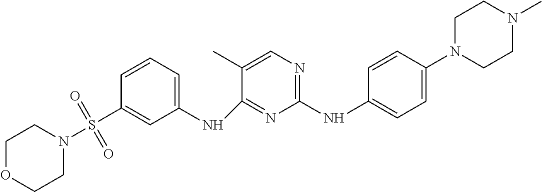 [SMILES: Cc1cnc(Nc2ccc(N3CCN(C)CC3)cc2)nc1Nc1cccc(S(=O)(=O)N2CCOCC2)c1] |
| M94 | 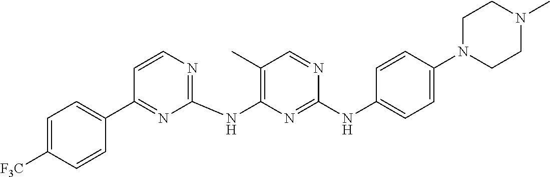 [SMILES: Cc1cnc(Nc2ccc(N3CCN(C)CC3)cc2)nc1Nc1nccc(-c2ccc(C(F)(F)F)cc2)n1] |
| B14 | 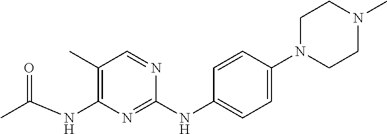 [SMILES: CC(=O)Nc1nc(Nc2ccc(N3CCN(C)CC3)cc2)ncc1C] |
| B2 | 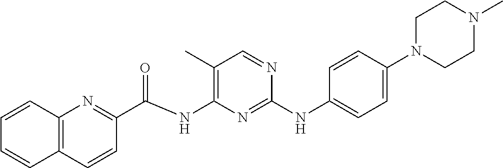 [SMILES: Cc1cnc(Nc2ccc(N3CCN(C)CC3)cc2)nc1NC(=O)c1ccc2ccccc2n1] |
| M24 | 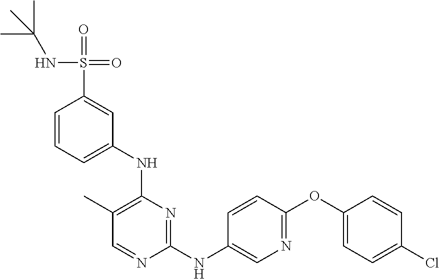 [SMILES: Cc1cnc(Nc2ccc(Oc3ccc(Cl)cc3)nc2)nc1Nc1cccc(S(=O)(=O)NC(C)(C)C)c1] |
| M25 | 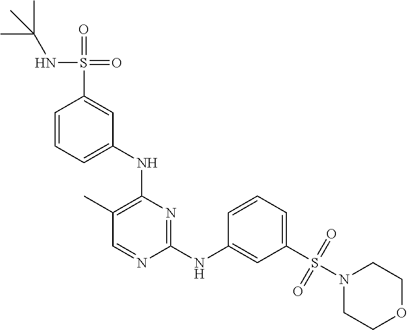 [SMILES: Cc1cnc(Nc2cccc(S(=O)(=O)N3CCOCC3)c2)nc1Nc1cccc(S(=O)(=O)NC(C)(C)C)c1] |
| M47 | 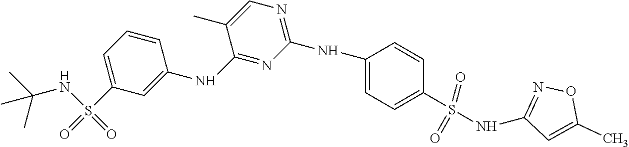 [SMILES: Cc1cc(NS(=O)(=O)c2ccc(Nc3ncc(C)c(Nc4cccc(S(=O)(=O)NC(C)(C)C)c4)n3)cc2)no1] |
| M48 | 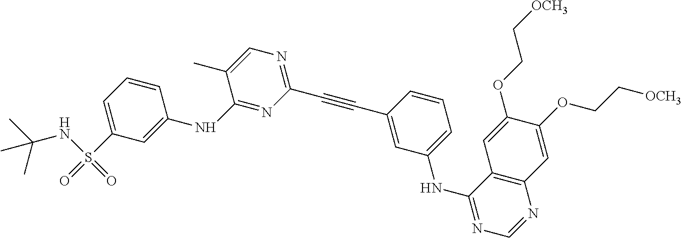 [SMILES: COCCOc1cc2ncnc(Nc3cccc(C#Cc4ncc(C)c(Nc5cccc(S(=O)(=O)NC(C)(C)C)c5)n4)c3)c2cc1OCCOC] |
| M55 | 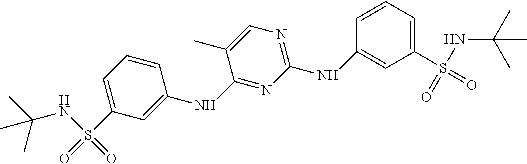 [SMILES: Cc1cnc(Nc2cccc(S(=O)(=O)NC(C)(C)C)c2)nc1Nc1cccc(S(=O)(=O)NC(C)(C)C)c1] |
| M57 | 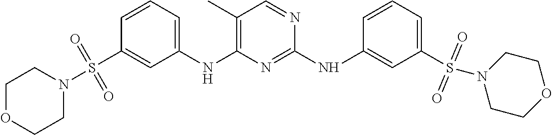 [SMILES: Cc1cnc(Nc2cccc(S(=O)(=O)N3CCOCC3)c2)nc1Nc1cccc(S(=O)(=O)N2CCOCC2)c1] |
| M58 | 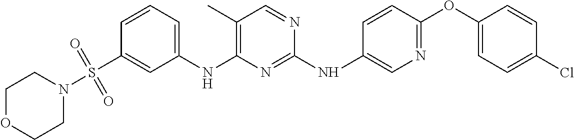 [SMILES: Cc1cnc(Nc2ccc(Oc3ccc(Cl)cc3)nc2)nc1Nc1cccc(S(=O)(=O)N2CCOCC2)c1] |
| M59 | 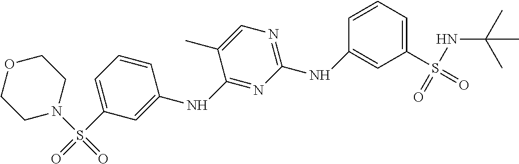 [SMILES: Cc1cnc(Nc2cccc(S(=O)(=O)NC(C)(C)C)c2)nc1Nc1cccc(S(=O)(=O)N2CCOCC2)c1] |
| M60 | 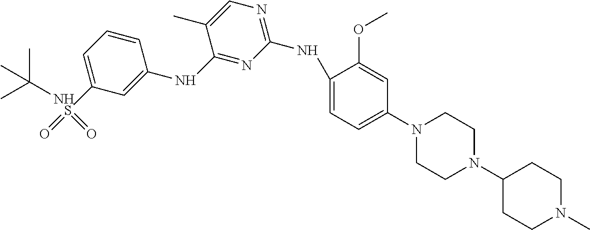 |
| M62 | 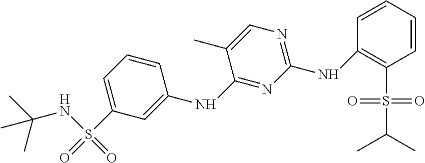 [SMILES: Cc1cnc(Nc2ccccc2S(=O)(=O)C(C)C)nc1Nc1cccc(S(=O)(=O)NC(C)(C)C)c1] |
| M64 | 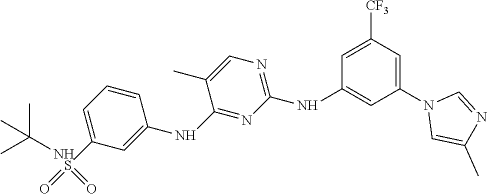 |
| M66 | 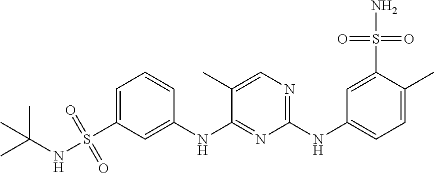 [SMILES: Cc1ccc(Nc2ncc(C)c(Nc3cccc(S(=O)(=O)NC(C)(C)C)c3)n2)cc1S(N)(=O)=O] |
| M69 | 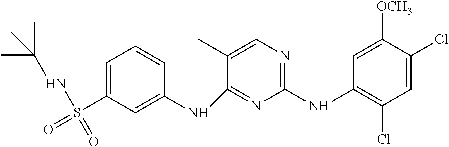 [SMILES: COc1cc(Nc2ncc(C)c(Nc3cccc(S(=O)(=O)NC(C)(C)C)c3)n2)c(Cl)cc1Cl] |
| M73 | 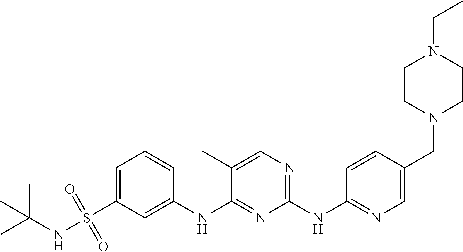 [SMILES: CCN1CCN(Cc2ccc(Nc3ncc(C)c(Nc4cccc(S(=O)(=O)NC(C)(C)C)c4)n3)nc2)CC1] |
| M79 | 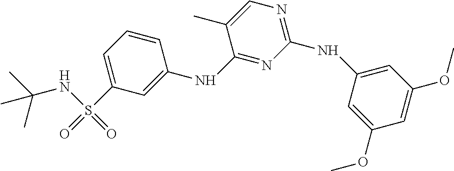 [SMILES: COc1cc(Nc2ncc(C)c(Nc3cccc(S(=O)(=O)NC(C)(C)C)c3)n2)cc(OC)c1] |
| M88 | 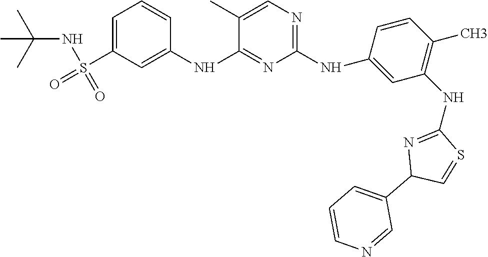 |
| M101 | 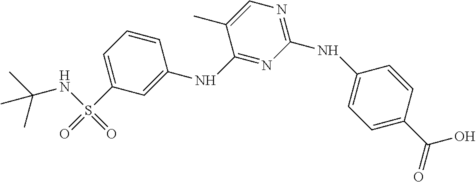 [SMILES: Cc1cnc(Nc2ccc(C(=O)O)cc2)nc1Nc1cccc(S(=O)(=O)NC(C)(C)C)c1] |
| M101A | 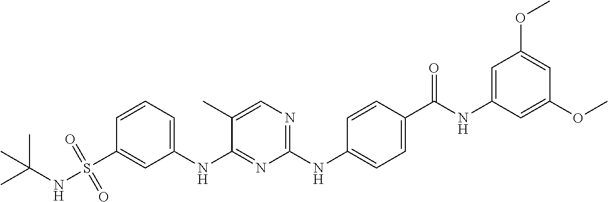 [SMILES: COc1cc(NC(=O)c2ccc(Nc3ncc(C)c(Nc4cccc(S(=O)(=O)NC(C)(C)C)c4)n3)cc2)cc(OC)c1] |
| M101B | 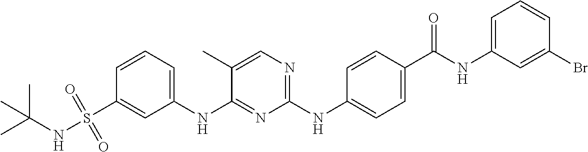 [SMILES: Cc1cnc(Nc2ccc(C(=O)Nc3cccc(Br)c3)cc2)nc1Nc1cccc(S(=O)(=O)NC(C)(C)C)c1] |
| M101D | 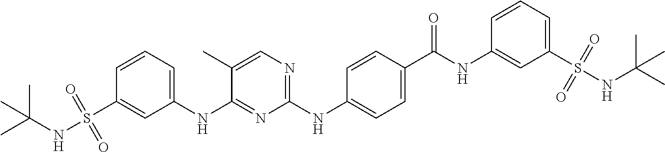 [SMILES: Cc1cnc(Nc2ccc(C(=O)Nc3cccc(S(=O)(=O)NC(C)(C)C)c3)cc2)nc1Nc1cccc(S(=O)(=O)NC(C)(C)C)c1] |
| M101F | 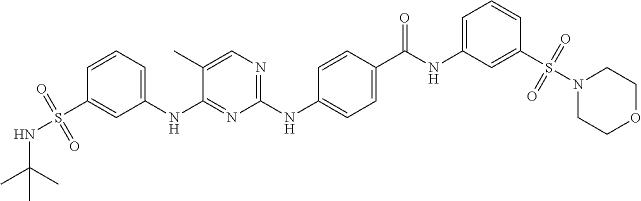 [SMILES: Cc1cnc(Nc2ccc(C(=O)Nc3cccc(S(=O)(=O)N4CCOCC4)c3)cc2)nc1Nc1cccc(S(=O)(=O)NC(C)(C)C)c1] |
| M101G | 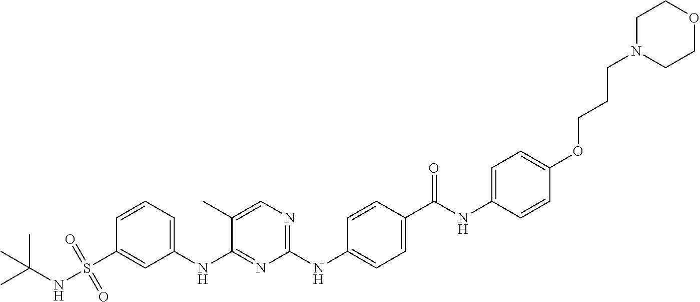 [SMILES: Cc1cnc(Nc2ccc(C(=O)Nc3ccc(OCCCN4CCOCC4)cc3)cc2)nc1Nc1cccc(S(=O)(=O)NC(C)(C)C)c1] |
| M101H | 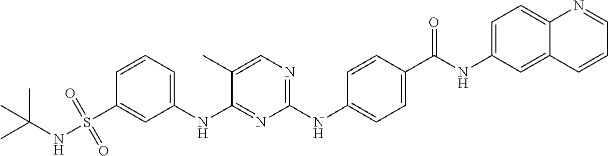 [SMILES: Cc1cnc(Nc2ccc(C(=O)Nc3ccc4ncccc4c3)cc2)nc1Nc1cccc(S(=O)(=O)NC(C)(C)C)c1] |
| M102 | 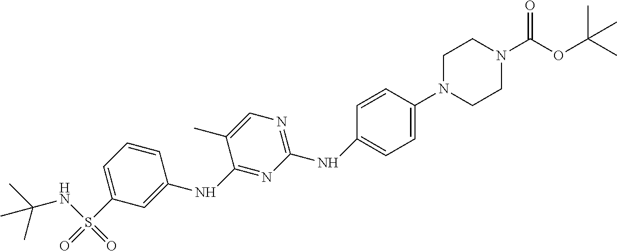 [SMILES: Cc1cnc(Nc2ccc(N3CCN(C(=O)OC(C)(C)C)CC3)cc2)nc1Nc1cccc(S(=O)(=O)NC(C)(C)C)c1] |
| M103 | 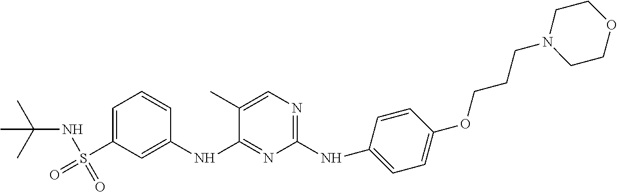 [SMILES: Cc1cnc(Nc2ccc(OCCCN3CCOCC3)cc2)nc1Nc1cccc(S(=O)(=O)NC(C)(C)C)c1] |
| M104 | 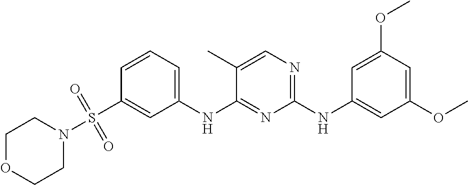 [SMILES: COc1cc(Nc2ncc(C)c(Nc3cccc(S(=O)(=O)N4CCOCC4)c3)n2)cc(OC)c1] |
| M105 | 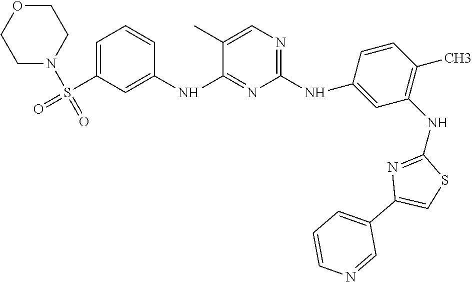 [SMILES: Cc1ccc(Nc2ncc(C)c(Nc3cccc(S(=O)(=O)N4CCOCC4)c3)n2)cc1Nc1nc(-c2cccnc2)cs1] |
| M106 | 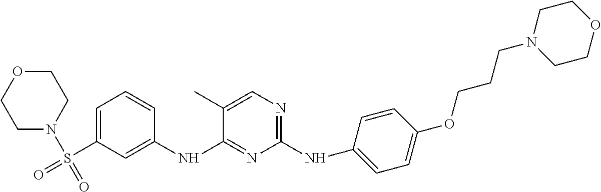 [SMILES: Cc1cnc(Nc2ccc(OCCCN3CCOCC3)cc2)nc1Nc1cccc(S(=O)(=O)N2CCOCC2)c1] |
| M108 | 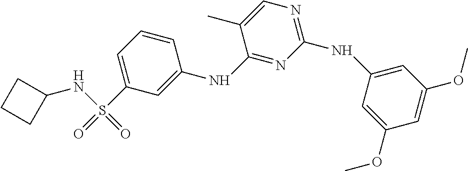 [SMILES: COc1cc(Nc2ncc(C)c(Nc3cccc(S(=O)(=O)NC4CCC4)c3)n2)cc(OC)c1] |

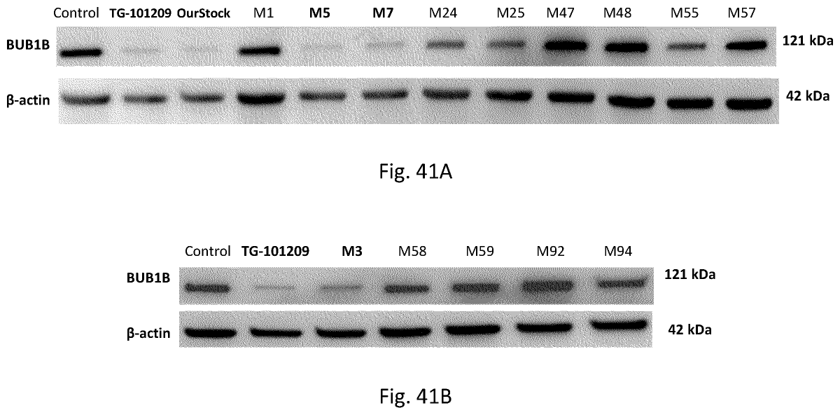

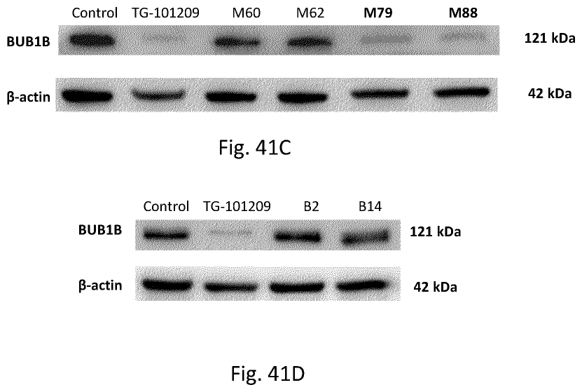

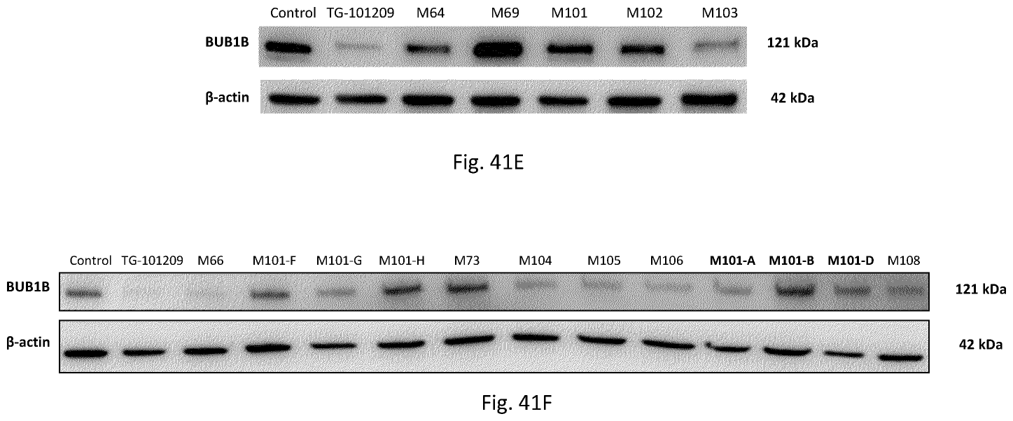

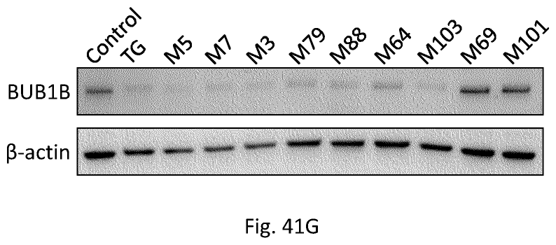

The effect of the synthesized molecules on the expression levels of BUB1B were evaluated by Western blot analysis, see FIG. 41A-41G. The figures show that the compounds M3, M5, M7, M64, M66, M79 and M88, in addition to TG-101209, had the ability to significantly lower the expression levels of BUB1B in the treated cells. Several other TG-101209 derivatives also showed the ability to lower the expression levels of BUB1B in the treated cells, such as compounds M101A, M101D, M101G and M103-M106, however, not to the same extent as the formerly mentioned.

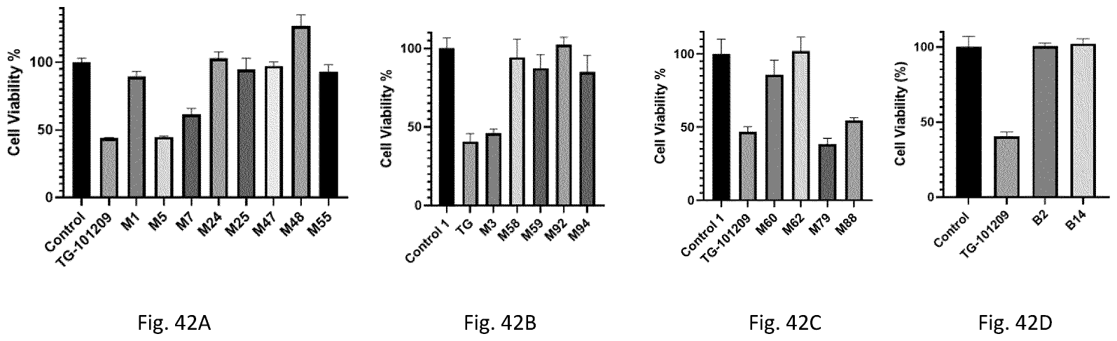

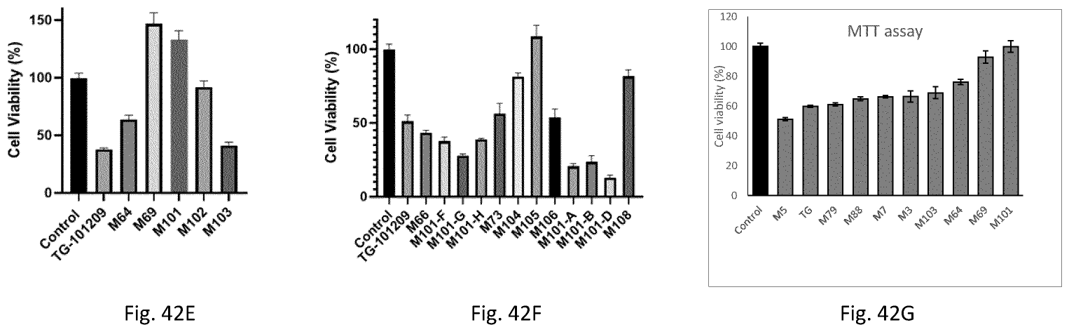

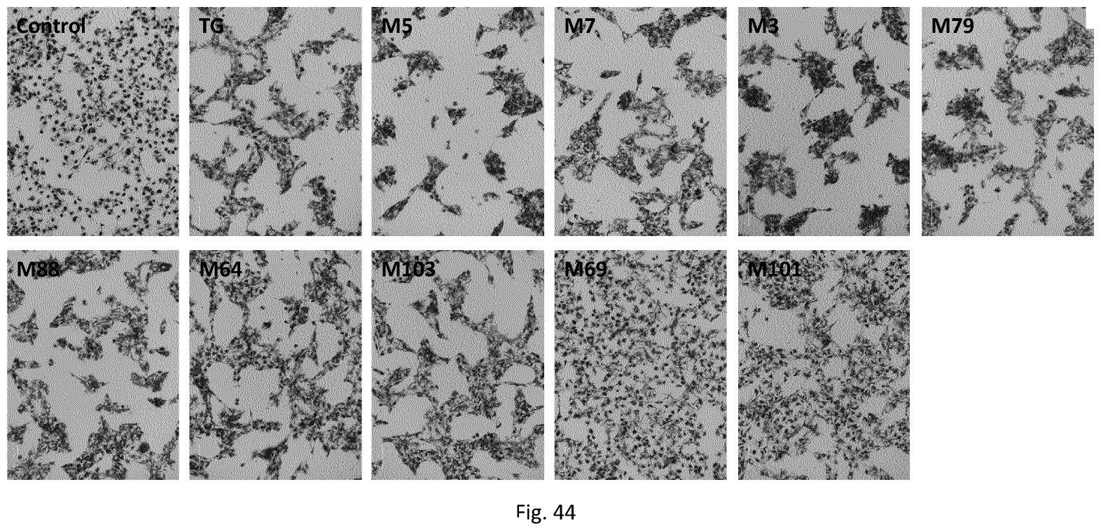

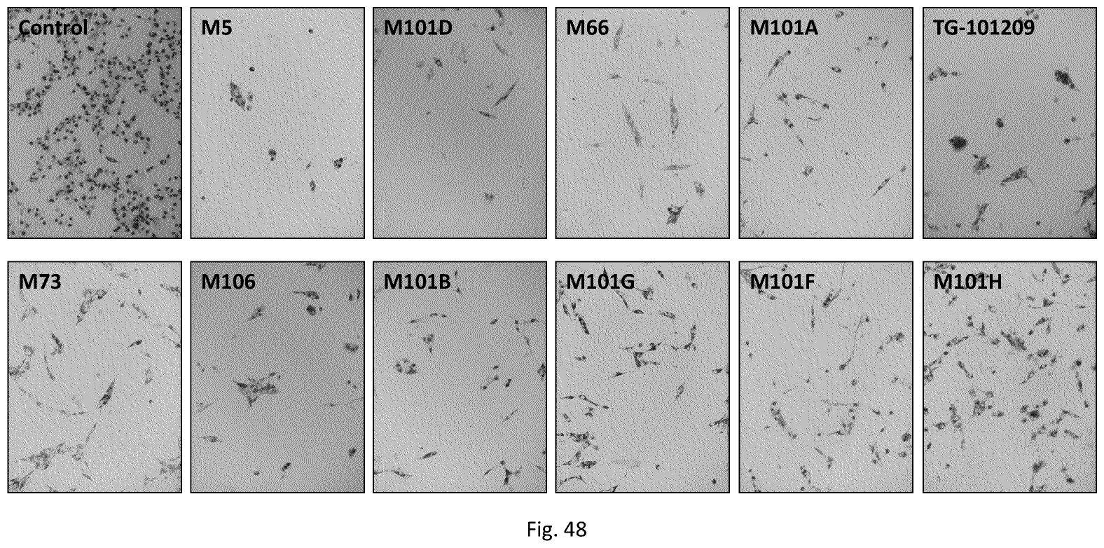

The cell viability of cells treated with 5 μM and 10 μM the TG-101209 and its derivatives was further evaluated using MTT assay, see FIG. 42A-42G, FIG. 44-45 and FIG. 48. Treatment with several of the synthesized TG-101209 derivatives resulted in a reduction of the cell viability.

The reduction in cell viability, when treating the cells with 5 μM of the TG-101209 derivatives, was the greatest for M101D, M101A, M101B, M101G, M101F, M101H, MS and M66. Treatment with these compounds resulted in a lower cell viability of Caki-1 cells than treatment with TG-101209.

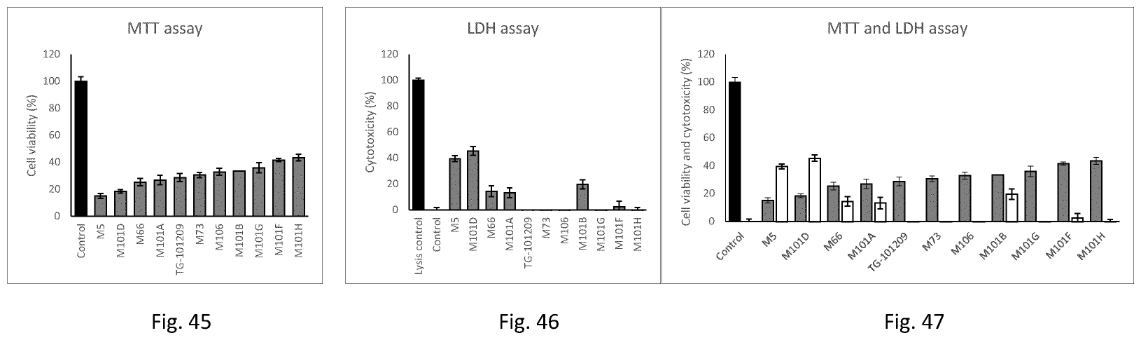

Furthermore, when treating the cells with 10 μM of the TG-101209 derivatives, see FIG. 45 and FIG. 47, treatment with MS, M101D, M66, M101A and TG-101209 had the most profound effect on the cell viability of Caki-1 cells, see Table 2. Treatment with M5 and M101D resulted in the greatest reduction of cell viability.

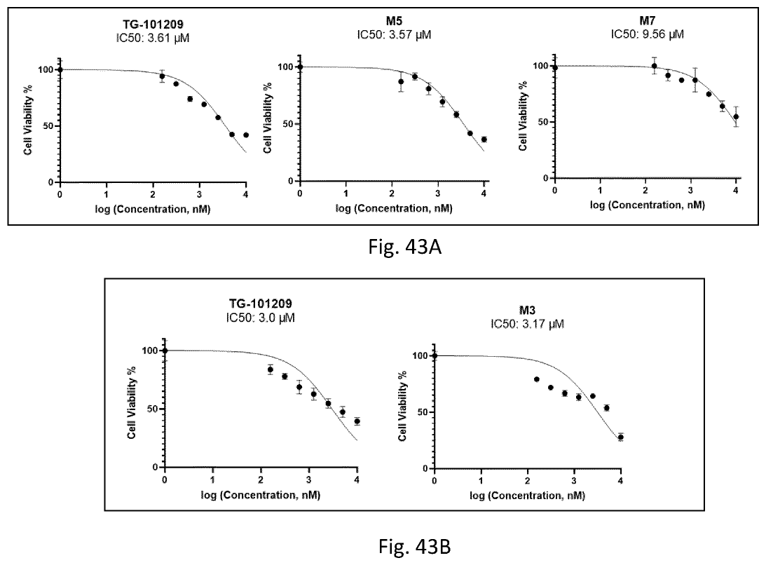

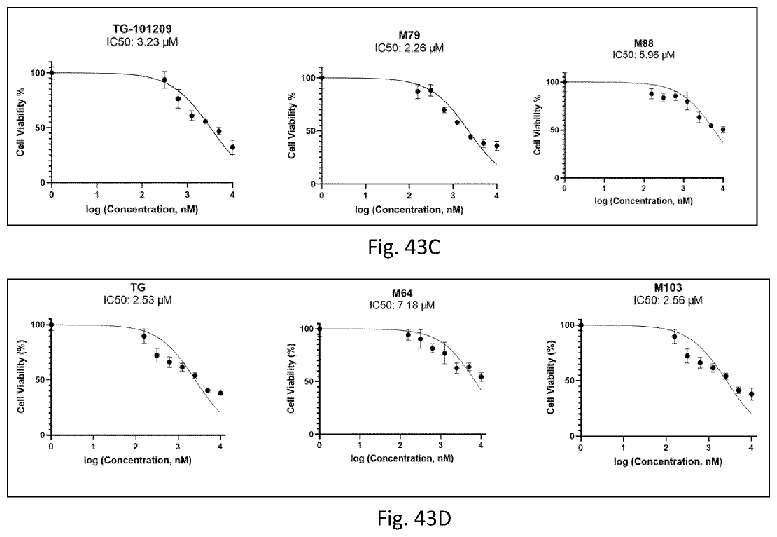

IC50 values were determined for TG-101209, M3, M5, M7, M79, M88, M64 and M103, see FIG. 43A-43D. TG-101209, M3, M5, M79 and M103 had IC50 values around 3 μM while the other evaluated target drugs had higher IC50 values.

The cytotoxicity of the target drugs was further assessed using LDH assay, see FIG. 46-47. Of the evaluated target drugs, M5, M101D, M66, M101A, M101B and M101F exhibited cytotoxicity wherein M5 and M101D exhibited the highest cytotoxicity, see Table 2.

Table 2 shows the ranking of the top five target drugs based on cell viability (MTT assay) and cytotoxicity (LDH assay) after treatment with 10 μM of target drugs for 1 day.

| Ranking | Cell viability (%) | Cytotoxicity (%) |
|---|---|---|
| 1 | M5 | M101D |
| 2 | M101D | M5 |
| 3 | M66 | M101B |
| 4 | M101A | M66 |
| 5 | TG-101209 | M101A |

In conclusion, several promising candidate drugs for the treatment of renal cell carcinoma, such as clear cell renal cell carcinoma (ccRCC), have been developed. Treatment of Caki-1 cells with the candidate drugs results in a reduction of the expression levels of BUB1B, reduction of the cell viability and potential cytotoxicity. Some of the promising candidate drugs include compounds TG-101209, M5, M66, M101A, M101B and M101D wherein M5 and M101D are the most promising candidate drug as they resulted the greatest reduction in cell viability when determined according to MTT assay and the greatest cytotoxicity when determined according to LDH assay.

### REFERENCES

[1] Motzer R J, Jonasch E, Boyle S, et al. NCCN Guidelines Insights: Kidney Cancer, Version 1.2021. J Natl Compr Canc Netw 2020; 18(9): 1160-70.
[2] National Comprehensive Cancer Network (NCCN). NCCN Clinical Practice Guidelines in Oncology. Kidney Cancer Version 1.2022. 2021. https://www.nccn.org/professionals/physician_gls/pdf/kidney.pdf (accessed Jul. 1, 2021).
[3] Hsieh J J, Purdue M P, Signoretti S, et al. Renal cell carcinoma. Nat Rev Dis Primers 2017; 3: 17009.
[4] Luo H, Li M, Yang M, Wu FX, Li Y, Wang J. Biomedical data and computational models for drug repositioning: a comprehensive review. Brief Bioinform 2021; 22(2): 1604-19.
[5] Turanli B, Altay O, Boren J, et al. Systems biology based drug repositioning for development of cancer therapy. Semin Cancer Biol 2021; 68: 47-58.
[6] Altay O, Mohammadi E, Lam S, et al. Current Status of COVID-19 Therapies and Drug Repositioning Applications. iScience 2020; 23(7): 101303.
[7] Nagaraj A B, Wang Q Q, Joseph P, et al. Using a novel computational drug-repositioning approach (DrugPredict) to rapidly identify potent drug candidates for cancer treatment. Oncogene 2018; 37(3): 403-14.
[8] Xuan P, Cao Y, Zhang T, Wang X, Pan S, Shen T. Drug repositioning through integration of prior knowledge and projections of drugs and diseases. Bioinformatics 2019; 35(20): 4108-19.
[9] Yang M, Wu G, Zhao Q, Li Y, Wang J. Computational drug repositioning based on multi-similarities bilinear matrix factorization. Brief Bioinform 2020.
[10] Zerbini L F, Bhasin M K, de Vasconcellos J F, et al. Computational repositioning and preclinical validation of pentamidine for renal cell cancer. Mol Cancer Ther 2014; 13(7): 1929-41.
[11] Koudijs K K M, Terwisscha van Scheltinga A G T, Bohringer S, Schimmel K J M, Guchelaar H J. Personalised drug repositioning for Clear Cell Renal Cell Carcinoma using gene expression. Sci Rep 2018; 8(1): 5250.
[12] Lamb J, Crawford E D, Peck D, et al. The Connectivity Map: using gene-expression signatures to connect small molecules, genes, and disease. Science 2006; 313(5795): 1929-35.
[13] Stathias V, Turner J, Koleti A, et al. LINCS Data Portal 2.0: next generation access point for perturbation-response signatures. Nucleic Acids Res 2020; 48(D1): D431-D9.
[14] Li X, Kim W, Juszczak K, et al. Stratification of patients with clear cell renal cell carcinoma to facilitate drug repositioning. iScience 2021; 24(7): 102722.
[15] Pornputtapong N, Nookaew I, Nielsen J. Human metabolic atlas: an online resource for human metabolism. Database (Oxford) 2015; 2015: bavo68.
[16] Mardinoglu A, Boren J, Smith U, Uhlen M, Nielsen J. Systems biology in hepatology: approaches and applications. Nat Rev Gastroenterol Hepatol 2018; 15(6): 365-77.
[17] Hanahan D, Weinberg R A. Hallmarks of cancer: the next generation. Cell 2011; 144(5): 646-74.
[18] Uhlen M, Zhang C, Lee S, et al. A pathology atlas of the human cancer transcriptome. Science 2017; 357(6352).
[19] Li X, Kim W, Arif M, et al. Discovery of Functional Alternatively Spliced PKM Transcripts in Human Cancers. Cancers (Basel) 2021; 13(2).
[20] Han JD. Understanding biological functions through molecular networks. Cell Res 2008; 18(2): 224-37.
[21] Stuart J M, Segal E, Koller D, Kim S K. A gene-coexpression network for global discovery of conserved genetic modules. Science 2003; 302(5643): 249-55.
[22] Pons P, Latapy M. Computing Communities in Large Networks Using Random Walks. Computer and Information Sciences—ISCIS 2005. Berlin, Heidelberg: Springer; 2005.
[23] Wu J, Xia X, Hu Y, Fang X, Orsulic S. Identification of Infertility-Associated Topologically Important Genes Using Weighted Co-expression Network Analysis. Front Genet 2021; 12: 580190.
[24] Bidkhori G, Benfeitas R, Klevstig M, et al. Metabolic network-based stratification of hepatocellular carcinoma reveals three distinct tumor subtypes. Proc Natl Acad Sci USA 2018; 115(50): E11874-E83.
[25] Palmer D, Fabris F, Doherty A, Freitas A A, de Magalhaes J P. Ageing transcriptome meta-analysis reveals similarities and differences between key mammalian tissues. Aging (Albany NY) 2021; 13(3): 3313-41.
[26] Bozhilova LV, Pardo-Diaz J, Reinert G, Deane CM. COGENT: evaluating the consistency of gene co-expression networks. Bioinformatics 2021; 37(13): 1928-9.
[27] Uhlen M, Fagerberg L, Hallstrom B M, et al. Proteomics. Tissue-based map of the human proteome. Science 2015; 347(6220): 1260419.
[28] Uhlen M, Bjorling E, Agaton C, et al. A human protein atlas for normal and cancer tissues based on antibody proteomics. Mol Cell Proteomics 2005; 4(12): 1920-32.
[29] Meyers R M, Bryan J G, McFarland J M, et al. Computational correction of copy number effect improves specificity of CRISPR-Cas9 essentiality screens in cancer cells. Nat Genet 2017; 49(12): 1779-84.
[30] Subramanian A, Narayan R, Corsello S M, et al. A Next Generation Connectivity Map: L1000 Platform and the First 1,000,000 Profiles. Cell 2017; 171(6): 1437-52 e17.
[31] Perlman Z E, Slack M D, Feng Y, Mitchison T J, Wu L F, Altschuler S J. Multidimensional drug profiling by automated microscopy. Science 2004; 306(5699): 1194-8.
[32] Cancer Genome Atlas Research N. Comprehensive molecular characterization of clear cell renal cell carcinoma. Nature 2013; 499(7456): 43-9.
[33] Brannon A R, Reddy A, Seiler M, et al. Molecular Stratification of Clear Cell Renal Cell Carcinoma by Consensus Clustering Reveals Distinct Subtypes and Survival Patterns. Genes Cancer 2010; 1(2): 152-63.
[34] Davenport J W, Fernandes E R, Harris L D, Neale G A, Goorha R. The mouse mitotic checkpoint gene bub1b, a novel bub1 family member, is expressed in a cell cycle-dependent manner. Genomics 1999; 55(1): 113-7.
[35] Whitfield M L, Sherlock G, Saldanha A J, et al. Identification of genes periodically expressed in the human cell cycle and their expression in tumors. Mol Biol Cell 2002; 13(6): 1977-2000.
[36] Grolmusz V K, Karaszi K, Micsik T, et al. Cell cycle dependent RRM2 may serve as proliferation marker and pharmaceutical target in adrenocortical cancer. Am J Cancer Res 2016; 6(9): 2041-53.
[37] Zhang K, Hu S, Wu J, et al. Overexpression of RRM2 decreases thrombspondin-1 and increases VEGF production in human cancer cells in vitro and in vivo: implication of RRM2 in angiogenesis. Mol Cancer 2009; 8: 11.
[38] Draetta G, Luca F, Westendorf J, Brizuela L, Ruderman J, Beach D. Cdc2 protein kinase is complexed with both cyclin A and B: evidence for proteolytic inactivation of MPF. Cell 1989; 56(5): 829-38.
[39] Petri E T, Errico A, Escobedo L, Hunt T, Basavappa R. The crystal structure of human cyclin B. Cell Cycle 2007; 6(11): 1342-9.
[40] Wu T, Zhang X, Huang X, Yang Y, Hua X. Regulation of cyclin B2 expression and cell cycle G2/m transition by menin. J Biol Chem 2010; 285(24): 18291-300.
[41] Corpet A, De Koning L, Toedling J, et al. Asfib, the necessary Asf1 isoform for proliferation, is predictive of outcome in breast cancer. EMBO J 2011; 30(3): 480-93.
[42] Sekino Y, Han X, Kobayashi G, et al. BUB1B Overexpression Is an Independent Prognostic Marker and Associated with CD44, p53, and P D-Li in Renal Cell Carcinoma. Oncology 2021; 99(4): 240-50.
[43] Osako Y, Yoshino H, Sakaguchi T, et al. Potential tumorsuppressive role of microRNA99a3p in sunitinibresistant renal cell carcinoma cells through the regulation of RRM2. Int J Oncol 2019; 54(5): 1759-70.
[44] Jiangqiao Z, Tao Q, Zhongbao C, et al. Anti-silencing function 1B histone chaperone promotes cell proliferation and migration via activation of the AKT pathway in clear cell renal cell carcinoma. Biochem Biophys Res Commun 2019; 511(1): 165-72.
[45] Peng R, Wang Y, Mao L, Fang F, Guan H. Identification of Core Genes Involved in the Metastasis of Clear Cell Renal Cell Carcinoma. Cancer Manag Res 2020; 12: 13437-49.
[46] Pardanani A, Hood J, Lasho T, et al. TG101209, a small molecule JAK2-selective kinase inhibitor potently inhibits myeloproliferative disorder-associated JAK2V617F and MPLW515L/K mutations. Leukemia 2007; 21(8): 1658-68.
[47] Ramakrishnan V, Kimlinger T, Haug J, et al. TG101209, a novel JAK2 inhibitor, has significant in vitro activity in multiple myeloma and displays preferential cytotoxicity for CD45+ myeloma cells. Am J Hematol 2010; 85(9): 675-86.
[48] Sun Y, Moretti L, Giacalone N J, et al. Inhibition of JAK2 signaling by TG101209 enhances radiotherapy in lung cancer models. J Thorac Oncol 2011; 6(4): 699-706.
[49] Galkin A V, Melnick J S, Kim S, et al. Identification of NVP-TAE684, a potent, selective, and efficacious inhibitor of NPM-ALK. ProcNat Acad Sci USA 2007; 104(1): 270-5.
[50] Duong H Q, Than V T, Nguyen H T, et al. Anaplastic lymphoma kinase inhibitor NVPTAE684 suppresses the proliferation of human pancreatic adenocarcinoma cells. Oncol Rep 2021; 45(4).
[51] Schonherr C, Ruuth K, Kamaraj S, et al. Anaplastic Lymphoma Kinase (ALK) regulates initiation of transcription of MYCN in neuroblastoma cells. Oncogene 2012; 31(50): 5193-200.
[52] Ye S, Zhang J, Shen J, et al. NVP-TAE684 reverses multidrug resistance (MDR) in human osteosarcoma by inhibiting P-glycoprotein (PGP1) function. Br J Pharmacol 2016; 173(3): 613-26.
[53] Mohan R, Hammers H J, Bargagna-Mohan P, et al. Withaferin A is a potent inhibitor of angiogenesis. Angiogenesis 2004; 7(2): 115-22.
[54] Um H J, Min K J, Kim D E, Kwon T K. Withaferin A inhibits JAK/STAT3 signaling and induces apoptosis of human renal carcinoma Caki cells. Biochem Biophys Res Commun 2012; 427(1): 24-9.
[55] Choi B Y, Kim B W. Withaferin-A Inhibits Colon Cancer Cell Growth by Blocking STAT3 Transcriptional Activity. J Cancer Prey 2015; 20(3): 185-92.
[56] Lee J, Hahm E R, Singh S V. Withaferin A inhibits activation of signal transducer and activator of transcription 3 in human breast cancer cells. Carcinogenesis 2010; 31(11): 1991-8.
[57] Yco L P, Mocz G, Opoku-Ansah J, Bachmann A S. Withaferin A Inhibits STAT3 and Induces Tumor Cell Death in Neuroblastoma and Multiple Myeloma. Biochem Insights 2014; 7: 1-13.
[58] Laubach J P, Moreau P, San-Miguel J F, Richardson P G. Panobinostat for the Treatment of Multiple Myeloma. Clin Cancer Res 2015; 21(21): 4767-73.
[59] Cha T L, Chuang M J, Wu S T, et al. Dual degradation of aurora A and B kinases by the histone deacetylase inhibitor LBH589 induces G2-M arrest and apoptosis of renal cancer cells. Clin Cancer Res 2009; 15(3): 840-50.
[60] Hainsworth J D, Infante J R, Spigel D R, Arrowsmith E R, Boccia R V, Burris H A. A phase I I trial of panobinostat, a histone deacetylase inhibitor, in the treatment of patients with refractory metastatic renal cell carcinoma. Cancer Invest 2011; 29(7): 451-5.
[61] Rausch M, Weiss A, Zoetemelk M, et al. Optimized Combination of HDACI and TKI Efficiently Inhibits Metabolic Activity in Renal Cell Carcinoma and Overcomes Sunitinib Resistance. Cancers (Basel) 2020; 12(11).
[62] Sato A, Asano T, Isono M, Ito K, Asano T. Panobinostat synergizes with bortezomib to induce endoplasmic reticulum stress and ubiquitinated protein accumulation in renal cancer cells. BMC Urol 2014; 14: 71.
[63] Tatlow P J, Piccolo S R. A cloud-based workflow to quantify transcript-expression levels in public cancer compendia. Sci Rep 2016; 6: 39259.
[64] Bray N L, Pimentel H, Melsted P, Pachter L. Near-optimal probabilistic RNA-seq quantification. Nat Biotechnol 2016; 34(5): 525-7.
[65] Colaprico A, Silva T C, Olsen C, et al. TCGAbiolinks: an R/Bioconductor package for integrative analysis of TCGA data. Nucleic Acids Res 2016; 44(8): e71.
[66] Sato Y, Yoshizato T, Shiraishi Y, et al. Integrated molecular analysis of clear-cell renal cell carcinoma. Nat Genet 2013; 45(8): 860-7.
[67] Quinlan A R, Hall I M. BEDTools: a flexible suite of utilities for comparing genomic features. Bioinformatics 2010; 26(6): 841-2.
[68] Ghandi M, Huang F W, Jane-Valbuena J, et al. Next-generation characterization of the Cancer Cell Line Encyclopedia. Nature 2019; 569(7757): 503-8.
[69] Yu G, Wang L G, Han Y, He Q Y. clusterProfiler: an R package for comparing biological themes among gene clusters. OMICS 2012; 16(5): 284-7.
[70] Csardi G, Nepusz T. The igraph software package for complex network research. InterJournal 2006; Complex Systems: 1695.
[71] Love M I, Huber W, Anders S. Moderated estimation of fold change and dispersion for RNA-seq data with DESea2. Genome Biol 2014; 15(12): 550.

1. A compound according to formula (I) 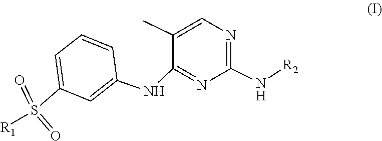
or a pharmaceutically acceptable salt or prodrug thereof wherein:
R₁ is selected from 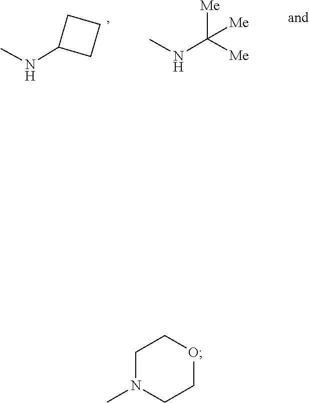
R₂ is 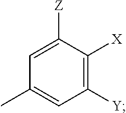
Z is —H, —OMe or —SO₂—NH₂;
X is —H, -Me, 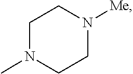
—NHC(O)—Ar or —C(O)NH—Ar wherein —Ar is an optionally substituted aryl; and
Y is —H, —OMe or 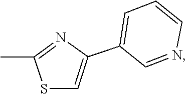
provided that the compound is not TG-101209.

2. The compound according to claim 1 wherein Y and Z is —H, X is —NHC(O)—Ar or —C(O)NH—Ar and the optionally substituted aryl, —Ar, has one or two rings.

3. The compound according to claim 2 wherein the optionally substituted aryl, —Ar, is chosen from the group of optionally substituted benzyl, naphthyl, quinolyl, indolyl or purinyl.

4. The compound according to claim 3 wherein —Ar is selected from the group consisting of

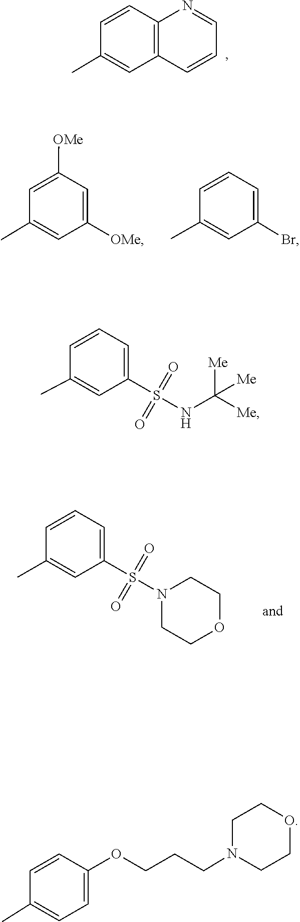

5. The compound according to claim 4 wherein —Ar is

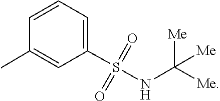

6. The compound according to claim 1 wherein X is -Me, Y is —H and Z is —SO₂NH₂.

7. The compound according to claim 1 wherein R₂ is

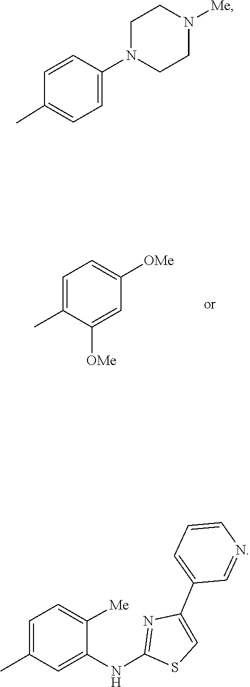 [SMILES: Cc1ccc(N2CCN(C)CC2)cc1 ; COc1ccc(C)c(OC)c1 ; Cc1ccc(C)c(Nc2nc(-c3cccnc3)cs2)c1]

8. The compound according to claim 5 wherein R₂ is

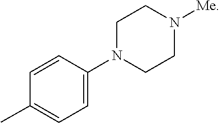

9. The compound according to claim 1 wherein R₁ is

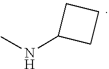

10. The compound according to claim 1 wherein R₁ is

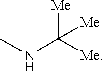

11. The compound according to claim 1 wherein R₁ is

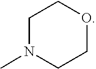

12. A pharmaceutical composition comprising a compound according to claim 1.

13. A method of treating a renal cell carcinoma comprising administering a compound according to claim 1.

14. (canceled)

15. The method of claim 13, further comprising administering a drug selected from the group consisting of tyrosine kinase inhibitors, mTOR inhibitors, monoclonal antibodies against VEGFe, panobinostat, NVP-TAE684 and withaferin-a.

16. The method of claim 13, wherein the renal cell carcinoma is clear cell renal cell carcinoma (ccRCC).

17. The method of claim 16, wherein the renal cell carcinoma is stage I-III ccRCC.

18. The method of claim 16, wherein the renal cell carcinoma is stage I or II ccRCC.

19. The method of claim 13, further comprising administering a drug selected from the group consisting of axitinib, sorafenib, pazopanib, sunitinib. Everolimus, tesirolimus, bevacizumab, panobinostat, NVP-TAE684 and withaferin-a.
